(19) United States
(12) Patent Application Publication
(10) Pub. No.: US 2026/0190855 A1
(43) Pub. Date: Jul. 2, 2026

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Wantian Xu, Beijing (CN); Zhihao Cui, Beijing (CN); Renjie Zheng, Beijing (CN); Xingxing Chai, Beijing (CN); Lei Zhao, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 19/431,037

(22) Filed: Dec. 23, 2025

(30) Foreign Application Priority Data
Dec. 31, 2024 (CN) 202411992767.7

(51) Int. Cl. H10K 85/60; C07B 59/00; C07D 221/20; C07D 471/10; C07D 491/107; C07D 495/10; H10K 50/18

(52) U.S. Cl. CPC H10K 85/626; C07B 59/002; C07D 221/20; C07D 471/10; C07D 491/107; C07D 495/10; H10K 85/6572; H10K 50/181

(57) ABSTRACT

Provided are an organic electroluminescent material and a device comprising the same. The organic electroluminescent material is compounds having a structure of Formula 1, and the compounds can be used in organic electroluminescent devices, for example, used as electron blocking materials. When applied in organic electroluminescent devices, these compounds can maintain a low voltage or significantly reduce the voltage, maintain high efficiency or significantly improve efficiency, and particularly, unexpectedly and dramatically enhance the device lifetime, thereby providing superior overall device performance. Further provided are an organic electroluminescent device comprising the compound and a compound composition comprising the compound.

### CROSS-REFERENCE TO RELATED APPLICATION(S)

This application claims priority to Chinese Patent Application No. 202411992767.7 filed on Dec. 31, 2024, the disclosure of which is incorporated herein by reference in its entirety.

### TECHNICAL FIELD

The present disclosure relates to compounds for organic electronic devices such as organic electroluminescent devices. More particularly, the present disclosure relates to a compound having a structure of Formula 1, an organic electroluminescent device comprising the compound and a compound composition comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modem organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

The performance of organic electroluminescent devices, such as efficiency and lifetime, is closely related to the balance of charge carriers concentration in the light-emitting layer. Compounds containing fluorene-spiroacridine structural fragments can be used as hole transport materials, electron blocking materials (light-emitting auxiliary materials) or host materials in electroluminescent devices, and several reports on such compounds have been published.

WO2014017844A1 discloses a compound having a structure of

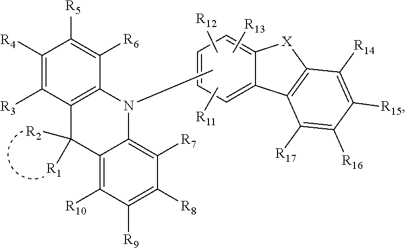

wherein X is selected from C, O, P, S, Se or Si, and specifically discloses compounds such as

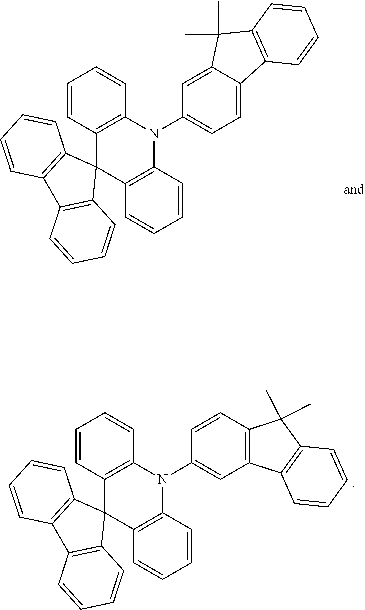 [SMILES: CC1(C)c2ccccc2-c2ccc(N3c4ccccc4C4(c5ccccc5-c5ccccc54)c4ccccc43)cc21 ; CC1(C)c2ccccc2-c2cc(N3c4ccccc4C4(c5ccccc5-c5ccccc54)c4ccccc43)ccc21]

The specific compounds containing a fluorene-spiroacridine structure disclosed in this application do not have additional fused ring structures on the acridine ring. This application neither discloses nor teaches compounds containing a fused acridine-spirofluorene structure or their effects on device performance. Furthermore, the compounds disclosed in this application are used as host materials in electroluminescent devices, and this application does not disclose or teach the impact of these compounds on device performance when used as other materials.

CN114790170A discloses a compound having a structure of

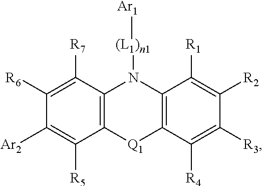

wherein Ar₁ is substituted or unsubstituted aryl having 6 to 30 ring-forming carbon atoms or substituted or unsubstituted heteroaryl having 2 to 30 ring-forming carbon atoms, and Ar₂ has a structure represented by

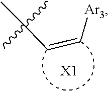

Q₁ is O, S, SO, SO₂, Se, CO, C(R₁₁)(R₁₂), Si(R₁₁)(R₁₂), Ge(R₁)(R₁₂), B(R₁₃), N(R₁₄), P(R₁₅), PO(R₁₆), PS(R₁₇) or a group represented by Formula 3,

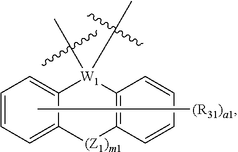

and discloses a compound

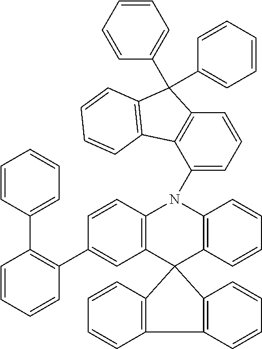 [SMILES: c1ccc(-c2ccccc2-c2ccc3c(c2)C2(c4ccccc4-c4ccccc42)c2ccccc2N3c2cccc3c2-c2ccccc2C3(c2ccccc2)c2ccccc2)cc1]

among the numerous specific structures disclosed. This application neither discloses nor teaches compounds containing an indenoacridine-spirofluorene structure joined to a fluorene/spirofluorene structure or their effects on device performance. Additionally, this application primarily focuses on the influence of Q₁ and/or Ar₁ on device performance, rather than the specific advantages of compounds containing an indenoacridine-spirofluorene structure joined to a fluorene/spirofluorene structure or their impact on device performance.

CN112480003A discloses a compound having a structure of

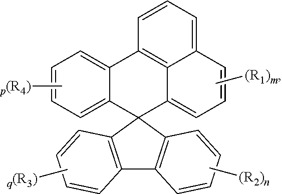

wherein at least one of R₁, R₂, R₃ or R₄ is a structure represented by

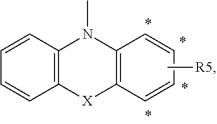

wherein X is —O—, —S—, —C(R₆)(R₇)— or —N(R₈)—; and R₅ is a structure represented by

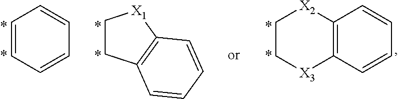

wherein X₁ is —O—, —S—, —C(R₉)(R₁₀)— or —N(R₁₁)—, and X₂ and X₃ are independently —O—, —S—, —C(R₁₂)(R₁₃)— or —N(R₁₄)—. This application discloses compounds such as

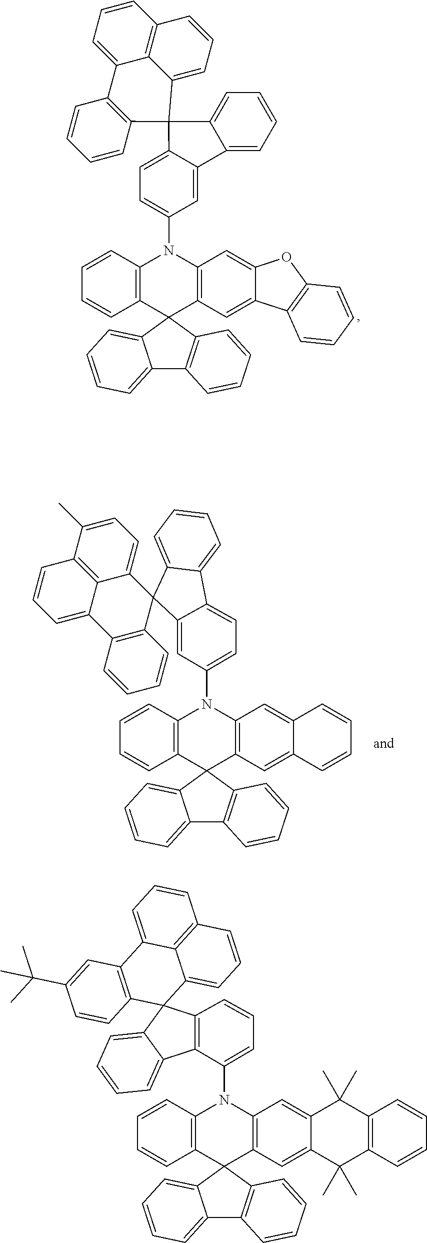

among the numerous disclosed specific structures. This application neither discloses nor teaches compounds containing an indenoacridine-spirofluorene structure joined to a fluorene/spirofluorene structure or their effects on device performance. Additionally, this application primarily emphasizes the influence of the benzo-spiroanthracene core skeleton on device performance, rather than the specific advantages of compounds containing an indenoacridine-spirofluorene structure joined to a fluorene/spirofluorene structure or their impact on device performance.

With the increasing demands in the industry for the performance of the organic electroluminescent devices, there remains a need for in-depth research and development of OLED materials having excellent performance such as lower voltage, higher efficiency and longer lifetime.

### SUMMARY

The present disclosure aims to provide a series of compounds represented by a structure of Formula 1, in which an indenoacridine-spirofluorene structural fragment is joined at specific positions to a fluorene/spirofluorene structural fragment, to solve at least part of the above problems. These compounds can be used in organic electroluminescent devices, for example, used as electron blocking materials. When applied in organic electroluminescent devices, these compounds can maintain a low voltage or significantly reduce the voltage, maintain high efficiency or significantly improve efficiency, and particularly, dramatically enhance the device lifetime, thereby providing superior overall device performance.

According to an embodiment of the present disclosure, a compound is disclosed, which has a structure represented by Formula 1:

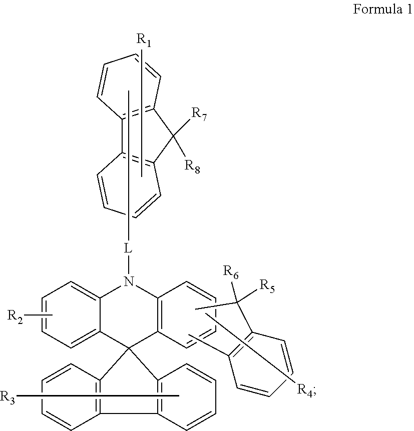

wherein
L is selected from a single bond, substituted or unsubstituted arylene having 6 to 30 carbon atoms, substituted or unsubstituted heteroarylene having 3 to 30 carbon atoms or a combination thereof;
R₁, R₂, R₃ and R₄ represent, at each occurrence identically or differently, mono-substitution, multiple substitutions or non-substitution;
R₁, R₂, R₃, R₅, R₆, R₇ and R₈ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₇ and R₈ can be optionally joined to form a ring.

According to another embodiment of the present disclosure, an organic electroluminescent device is disclosed, which comprises an anode, a cathode and an organic layer disposed between the anode and the cathode, wherein the organic layer comprises the compound in the preceding embodiment.

According to another embodiment of the present disclosure, a compound composition is further disclosed, which comprises the compound in the preceding embodiment.

The present disclosure discloses a series of compounds represented by a structure of Formula 1, in which an indenoacridine-spirofluorene structure is joined at specific positions to a fluorene/spirofluorene structure. When applied in organic electroluminescent devices, these compounds can significantly improve the performance of the organic electroluminescent devices, for example, can maintain a low voltage or significantly reduce the voltage, maintain high efficiency or significantly improve efficiency, and particularly, dramatically enhance the device lifetime, thereby providing superior overall device performance.

### BRIEF DESCRIPTION OF DRAWINGS

FIG. 1 is a schematic diagram of an organic light-emitting device that may comprise a compound and a compound composition disclosed herein.

FIG. 2 is a schematic diagram of another organic light-emitting device that may comprise a compound and a compound composition disclosed herein.

### DETAILED DESCRIPTION

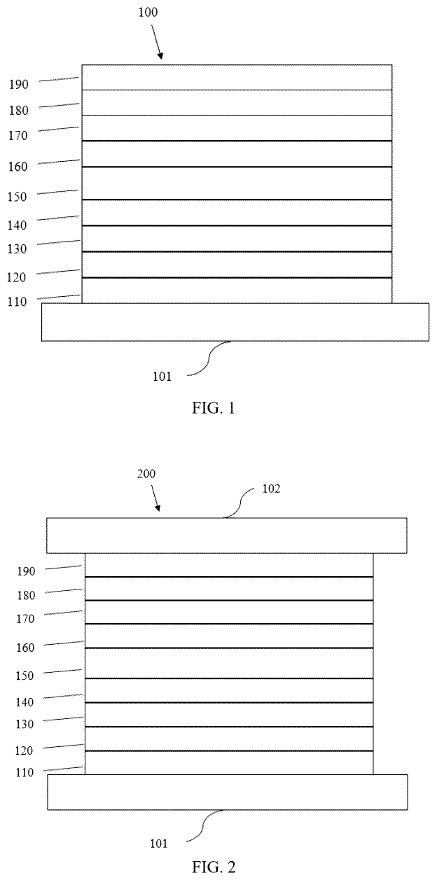

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light-emitting device 200 without limitation. FIG. 2 differs from FIG. 1 in that the organic light-emitting device includes a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e., P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as the temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in a small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, an n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbomyl, 2-norbomyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbomenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g., phenyl, phenylene, naphthyl, dibenzofuryl) or as if it were the whole molecule (e.g., benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to their enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions, etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

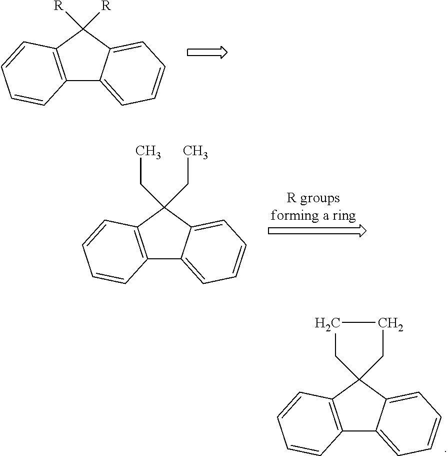

The expression that adjacent substituents can be optionally joined to form a ring is also intended to mean that two substituents bonded to carbon atoms which are directly bonded to each other are joined to each other via a chemical bond to form a ring, which can be exemplified by the following formula:

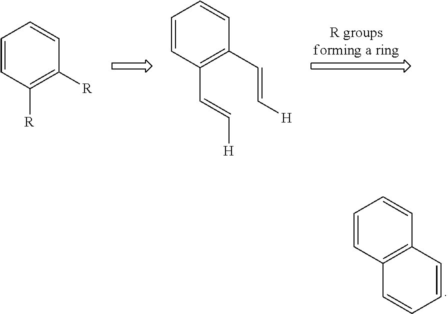

The expression that adjacent substituents can be optionally joined to form a ring is also intended to mean that two substituents bonded to further distant carbon atoms are joined to each other via a chemical bond to form a ring, which can be exemplified by the following formula:

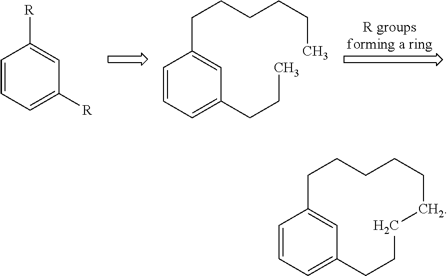

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

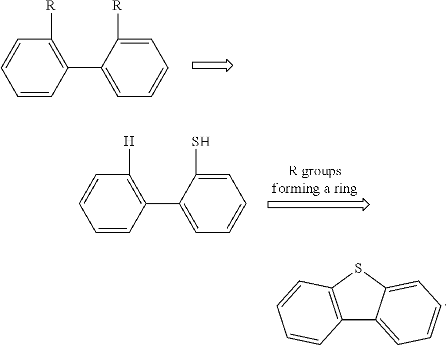

According to an embodiment of the present disclosure, a compound is disclosed, which has a structure represented by Formula 1:

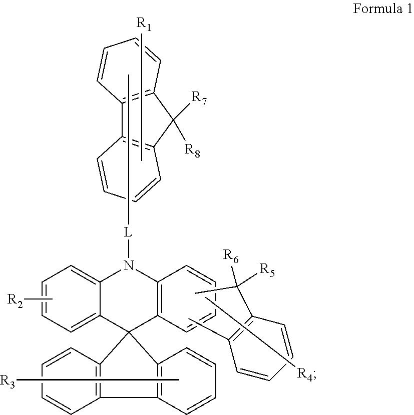

wherein
L is selected from a single bond, substituted or unsubstituted arylene having 6 to 30 carbon atoms, substituted or unsubstituted heteroarylene having 3 to 30 carbon atoms or a combination thereof;
R₁, R₂, R₃ and R₄ represent, at each occurrence identically or differently, mono-substitution, multiple substitutions or non-substitution;
R₁, R₂, R₃, R₅, R₆, R₇ and R₈ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₇ and R₈ can be optionally joined to form a ring.

In the present disclosure, the expression that adjacent substituents R₁, R₂, R₃, R₄, R₅, R₆, R₇ and R₈ can be optionally joined to form a ring is intended to mean that any one or more of groups of adjacent substituents, such as two substituents R₁, two substituents R₂, two substituents R₃, two substituents R₄, substituents R₂ and R₃, substituents R₃ and R₄, substituents R₅ and R₆, and substituents R₇ and R₈, can be joined to form a ring. Obviously, it is also possible that none of these substituents are joined to form a ring.

According to an embodiment of the present disclosure, the compound has a structure represented by Formula 2:

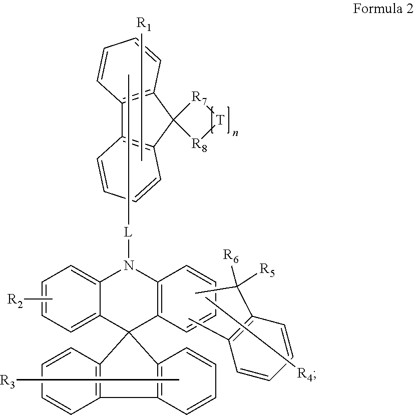

wherein T is selected from a single bond, O, S or CR₉R₁₀;
n is selected from 0 or 1;
L is selected from a single bond, substituted or unsubstituted arylene having 6 to 30 carbon atoms, substituted or unsubstituted heteroarylene having 3 to 30 carbon atoms or a combination thereof;
R₁, R₂, R₃ and R₄ represent, at each occurrence identically or differently, mono-substitution, multiple substitutions or non-substitution;
R₁, R₂, R₃, R₅, R₆, R₇, R₈, R₉ and R₁₀ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₉ and R₁₀ can be optionally joined to form a ring.

In this embodiment, n being selected from 0 represents that R₇ and R₈ are not joined to form a ring; n being selected from 1 represents that R₇ and R₈ are joined to form a ring through T; when R₇ and R₈ are joined to form a ring through T, the structure of the ring containing R₇, R₈ and T includes, but is not limited to:

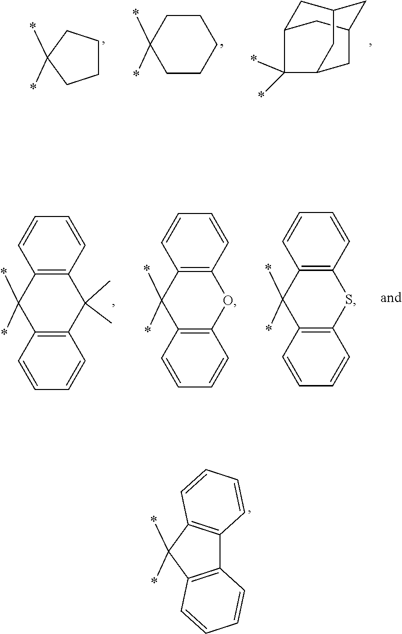

and the structures may be optionally substituted.

In the present disclosure, the expression that adjacent substituents R₁, R₂, R₃, R₄, R₅, R₆, R₉ and R₁₀ can be optionally joined to form a ring is intended to mean that any one or more of groups of adjacent substituents, such as two substituents R₁, two substituents R₂, two substituents R₃, two substituents R₄, substituents R₂ and R₃, substituents R₃ and R₄, substituents R₅ and R₆, and substituents R₉ and R₁₀, can be joined to form a ring. Obviously, it is also possible that none of these substituents are joined to form a ring.

In the present disclosure, any adjacent substituents R₄ are not joined to form a ring.

In the present disclosure, any adjacent substituents R₄ are not joined to form a benzene ring.

In an embodiment of the present disclosure, R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; wherein when R₂ is selected from substituted or unsubstituted aryl having 6 to 30 carbon atoms, R₂ is not biphenyl-2-yl.

According to an embodiment of the present disclosure, at least two adjacent substituents R₂ are joined to form a ring.

According to an embodiment of the present disclosure, at least two adjacent substituents R₂ are joined to form a substituted or unsubstituted aromatic ring having 6 to 30 carbon atoms or a substituted or unsubstituted heteroaromatic ring having 3 to 30 carbon atoms.

According to an embodiment of the present disclosure, at least two adjacent substituents R₂ are joined to form a substituted or unsubstituted aromatic ring having 6 to 12 carbon atoms.

According to an embodiment of the present disclosure, at least two adjacent substituents R₂ are joined to form a substituted or unsubstituted benzene ring or a substituted or unsubstituted indene ring.

According to an embodiment of the present disclosure, the compound has a structure represented by Formula 2-1 or Formula 2-2:

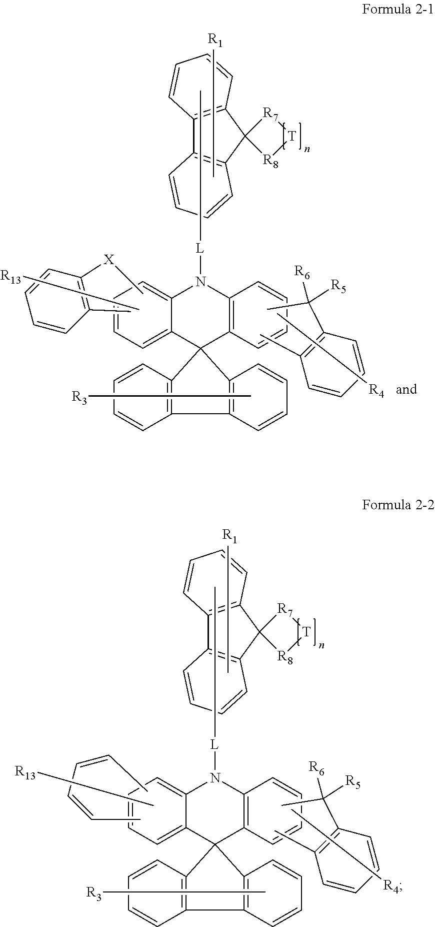

wherein T is selected from a single bond, O, S or CR₉R₁₀;
n is selected from 0 or 1;
X is selected from O, S or CR₁₁R₁₂;
L is selected from a single bond, substituted or unsubstituted arylene having 6 to 30 carbon atoms, substituted or unsubstituted heteroarylene having 3 to 30 carbon atoms or a combination thereof;
R₁, R₃, R₄ and R₁₃ represent, at each occurrence identically or differently, mono-substitution, multiple substitutions or non-substitution;
R₁, R₃, R₅, R₆, R₇, R₈, R₉, R₁₀, R₁₁, R₁₂ and R₁₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₃, R₄, R₅, R₆, R₉, R₁₀, R₁₁, R₁₂ and R₁₃ can be optionally joined to form a ring.

In the present disclosure, the expression that adjacent substituents R₁, R₃, R₄, R₅, R₆, R₉, R₁₀, R₁₁, R₁₂ and R₁₃ can be optionally joined to form a ring is intended to mean that any one or more of groups of adjacent substituents, such as two substituents R₁, two substituents R₃, two substituents R₄, two substituents R₁₃, substituents R₃ and R₄, substituents R₃ and R₁₃, substituents R₅ and R₆, substituents R₉ and R₁₀, and substituents R₁₁ and R₁₂, can be joined to form a ring. Obviously, it is also possible that none of these substituents are joined to form a ring.

According to an embodiment of the present disclosure, the compound has a structure represented by any one of Formula 3-1 to Formula 3-9:

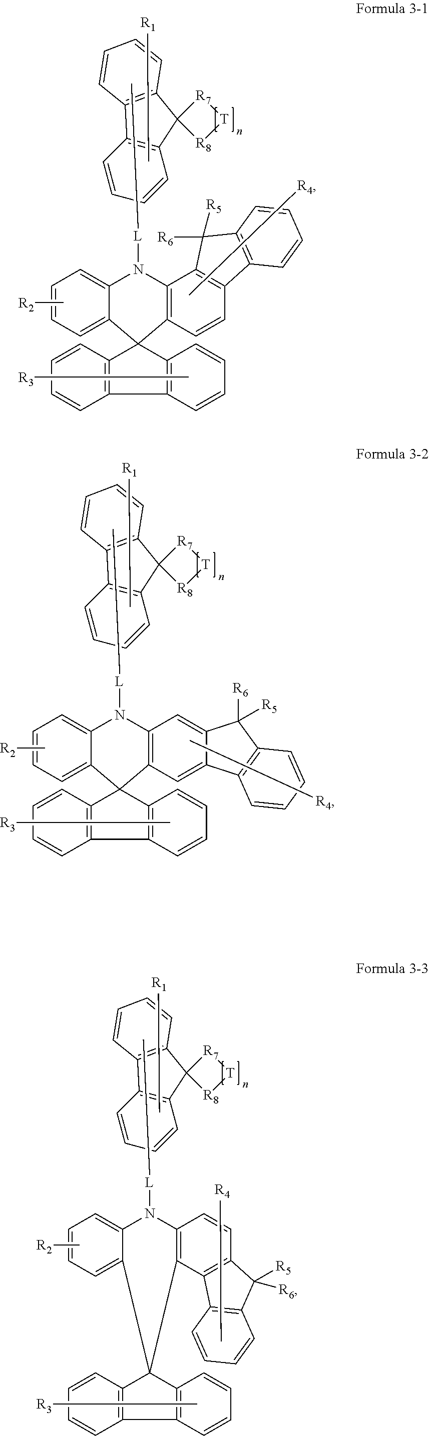

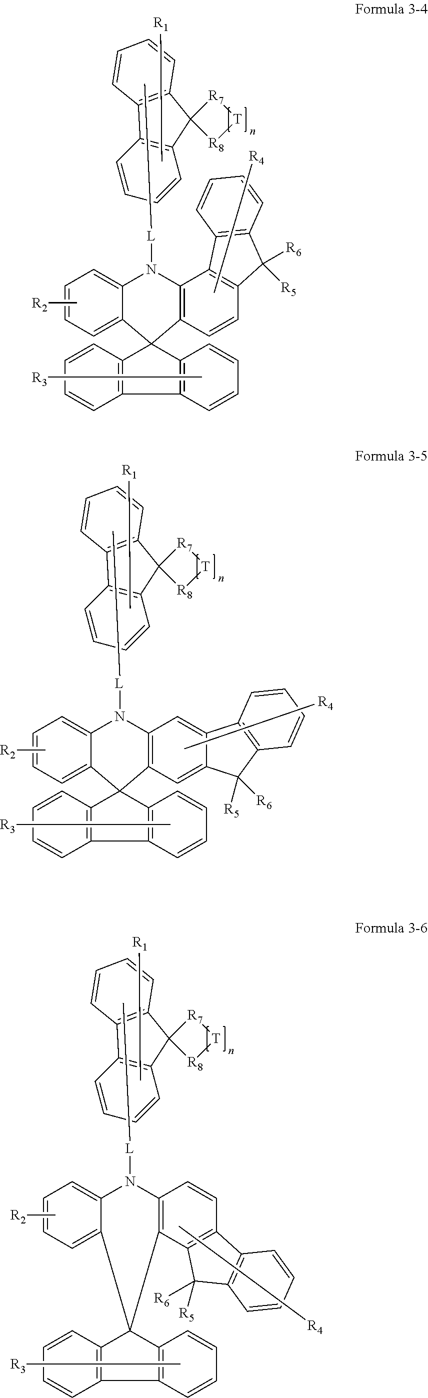

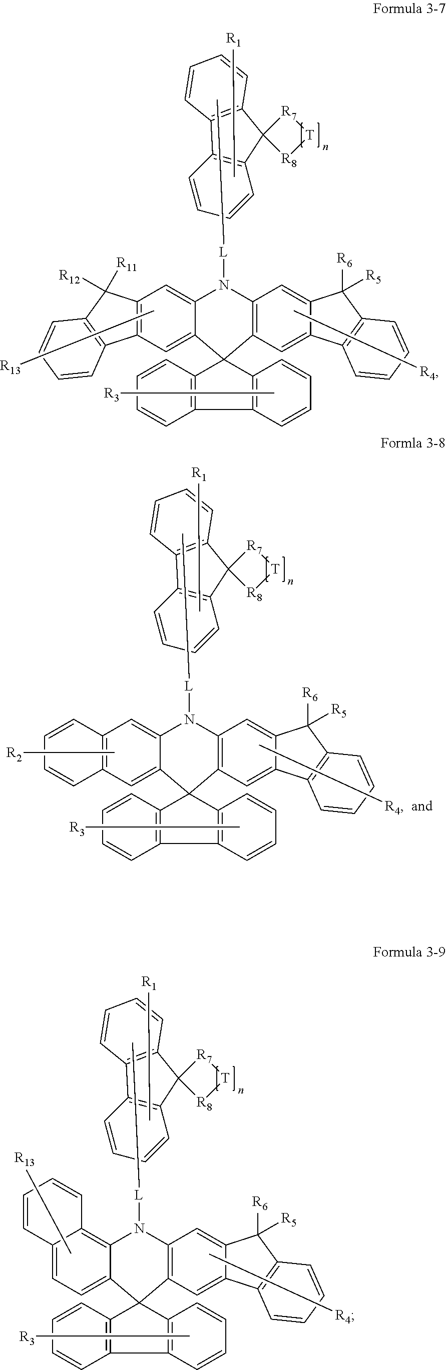

wherein T is selected from a single bond, O, S or CR₉R₁₀;
n is selected from 0 or 1;
R₁, R₂, R₃, R₄ and R₁₃ represent, at each occurrence identically or differently, mono-substitution, multiple substitutions or non-substitution;
R₁, R₂, R₃, R₅, R₆, R₇, R₈, R₉, R₁₀, R₁₁, R₁₂, and R₁₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₉, R₁₀, R₁₁, R₁₂, and R₁₃ can be optionally joined to form a ring.

In the present disclosure, the expression that adjacent substituents R₁, R₂, R₃, R₄, R₅, R₆, R₉, R₁₀, R₁₁, R₁₂, and R₁₃ can be optionally joined to form a ring is intended to mean that any one or more of groups of adjacent substituents, such as two substituents R₁, two substituents R₂, two substituents R₃, two substituents R₄, two substituents R₁₃, substituents R₂ and R₃, substituents R₃ and R₄, substituents R₃ and R₁₃, substituents R₅ and R₆, substituents R₉ and R₁₀, and substituents R₁₁ and R₁₂, can be joined to form a ring. Obviously, it is also possible that none of these substituents are joined to form a ring.

According to an embodiment of the present disclosure, the compound has a structure represented by Formula 3-2, Formula 3-5, Formula 3-7, Formula 3-8 or Formula 3-9.

According to an embodiment of the present disclosure, the L is selected from a single bond or substituted or unsubstituted arylene having 6 to 30 carbon atoms.

According to an embodiment of the present disclosure, the L is selected from a single bond, substituted or unsubstituted phenylene, substituted or unsubstituted biphenylylene, substituted or unsubstituted naphthylene or substituted or unsubstituted fluorenylidene.

According to an embodiment of the present disclosure, the L is selected from a single bond or substituted or unsubstituted phenylene.

According to an embodiment of the present disclosure, the R₁, R₂, R₃, R₄, R₅, R₆, R₇, R₈, R₉, R₁₀, R₁₁, R₁₂ and R₁₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, the R₁, R₂, R₃, R₄, R₅, R₆, R₇, R₈, R₉, R₁₀, R₁₁, R₁₂ and R₁₃ are, at each occurrence identically or differently, selected from the group consisting of: hydrogen, deuterium, fluorine, substituted or unsubstituted methyl, substituted or unsubstituted ethyl, substituted or unsubstituted isopropyl, substituted or unsubstituted tert-butyl, substituted or unsubstituted cyclopentyl, substituted or unsubstituted cyclohexyl, substituted or unsubstituted adamantyl, substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted dibenzofuryl, substituted or unsubstituted dibenzothienyl, substituted or unsubstituted dibenzoselenophenyl, substituted or unsubstituted carbazolyl, substituted or unsubstituted phenanthryl, substituted or unsubstituted anthryl, and combinations thereof.

According to an embodiment of the present disclosure, at least one of the R₁, R₂, R₃, R₄ or R₁₃ is, at each occurrence identically or differently, selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, at least one of the R₁, R₂, R₃, R₄ or R₁₃ is, at each occurrence identically or differently, selected from the group consisting of: deuterium, substituted or unsubstituted methyl, substituted or unsubstituted ethyl, substituted or unsubstituted isopropyl, substituted or unsubstituted tert-butyl, substituted or unsubstituted cyclopentyl, substituted or unsubstituted cyclohexyl, substituted or unsubstituted adamantyl, substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted dibenzofuryl, substituted or unsubstituted dibenzothienyl, substituted or unsubstituted dibenzoselenophenyl, substituted or unsubstituted carbazolyl, substituted or unsubstituted phenanthryl, substituted or unsubstituted anthryl, and combinations thereof.

According to an embodiment of the present disclosure, n is selected from 0, and the R₇ and R₈ are, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, n is selected from 0, and the R₇ and R₈ are, at each occurrence identically or differently, selected from the group consisting of: substituted or unsubstituted alkyl having 1 to 20 carbon atoms, substituted or unsubstituted aryl having 6 to 30 carbon atoms, and combinations thereof.

According to an embodiment of the present disclosure, n is selected from 0, and the R₇ and R₈ are, at each occurrence identically or differently, selected from substituted or unsubstituted methyl or substituted or unsubstituted phenyl.

According to an embodiment of the present disclosure, n is selected from 1, and T is selected from a single bond or CR₉R₁₀.

According to an embodiment of the present disclosure, n is selected from 1, T is selected from a single bond, the R₇ and R₈ are, at each occurrence identically or differently, selected from the group consisting of: substituted or unsubstituted alkylene having 1 to 20 carbon atoms, substituted or unsubstituted cycloalkylene having 3 to 20 ring carbon atoms, substituted or unsubstituted arylene having 6 to 30 carbon atoms, substituted or unsubstituted heteroarylene having 3 to 30 carbon atoms, and combinations thereof.

According to an embodiment of the present disclosure, n is selected from 1, T is selected from a single bond, and the R₇ and R₈ are selected from substituted or unsubstituted phenylene.

According to an embodiment of the present disclosure, the compound is selected from the group consisting of Compound A1 to Compound A124, Compound B1 to Compound B92, and Compound C1 to Compound C98, wherein the specific structures of Compound A1 to Compound A124, Compound B1 to Compound B92, and Compound C1 to Compound C98 are referred to claim 10.

According to an embodiment of the present disclosure, hydrogens in the structures of Compound A1 to Compound A124, Compound B1 to Compound B92, and Compound C1 to Compound C98 can be partially or fully substituted with deuterium.

According to an embodiment of the present disclosure, an organic electroluminescent device is disclosed, which comprises an anode, a cathode and an organic layer disposed between the anode and the cathode, wherein the organic layer comprises the compound in any one of the preceding embodiments.

According to an embodiment of the present disclosure, the organic layer is an electron blocking layer, a hole injection layer, a hole transport layer or a light-emitting layer.

According to an embodiment of the present disclosure, the organic layer is an electron blocking layer, and the compound is an electron blocking material.

According to an embodiment of the present disclosure, the thickness of the electron blocking layer ranges from 1 nm to 800 nm.

According to an embodiment of the present disclosure, the organic layer is a light-emitting layer, and the compound is a host material.

According to an embodiment of the present disclosure, the light-emitting layer comprises a phosphorescent material.

According to an embodiment of the present disclosure, the organic electroluminescent device emits red light.

According to an embodiment of the present disclosure, the organic electroluminescent device emits green light.

According to an embodiment of the present disclosure, an organic electroluminescent device is disclosed, which comprises an anode, a cathode, a hole injection layer, a hole transport layer, an electron blocking layer and a light-emitting layer, wherein the electron blocking layer comprises the compound in any one of the preceding embodiments.

According to an embodiment of the present disclosure, the electron blocking layer is in direct contact with the hole transport layer, and the electron blocking layer is in direct contact with the light-emitting layer.

According to an embodiment of the present disclosure, the hole transport layer comprises a hole transport material, wherein the hole transport material comprises a mono-triarylamine compound or a bis-triarylamine compound.

According to an embodiment of the present disclosure, a compound composition is disclosed, which comprises the compound in any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compounds disclosed herein may be used in combination with a wide variety of light-emitting dopants, hosts, transporting layers, blocking layers, injection layers, electrodes, and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### MATERIAL SYNTHESIS EXAMPLE

The method for preparing the compound of the present disclosure is not limited. Typically, the following compounds are used as examples without limitation, and the synthesis routes and preparation methods thereof are described below.

### Synthesis Example 1: Synthesis of Compound A1

### Step 1: Synthesis of Intermediate A

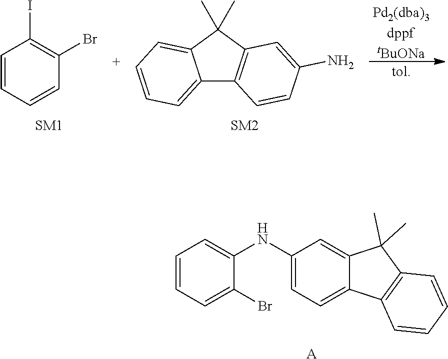

Under an atmosphere of N₂, Intermediate SM1 (27 g, 95.6 mmol), Intermediate SM2 (20 g, 95.6 mmol), sodium tert-butoxide (18.4 g, 191.2 mmol), Pd₂(dba)₃ (1.75 g, 1.91 mmol), dppf (1,1′-bis(diphenylphosphino)ferrocene, 3.18 g, 5.74 mmol) and toluene (1000 mL) were added to a 2000 mL reaction flask in sequence. The temperature was raised to 120° C., and the mixture reacted for 3 h. After TLC showed that the reaction was completed, the temperature was cooled to room temperature, and the reaction solution was filtered through Celite, concentrated under reduced pressure and purified through column chromatography to obtain Intermediate A as a white solid (30 g, with a yield of 86.2%).

### Step 2: Synthesis of Intermediate B

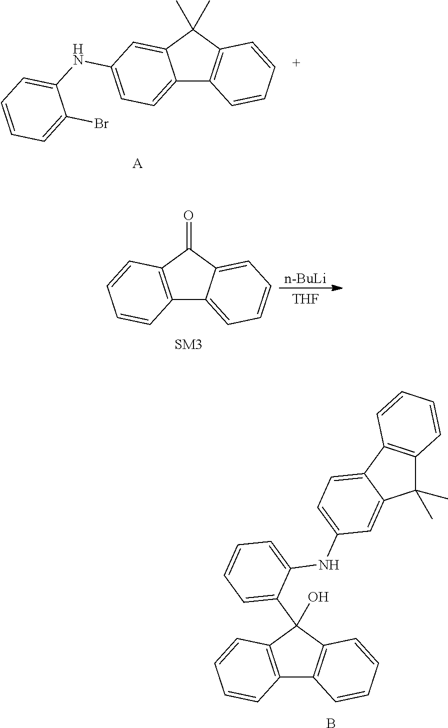

Under an atmosphere of N₂, Intermediate A (30 g, 82.35 mmol) and THF (240 mL) were added to a 500 mL two-necked flask. After the temperature was lowered to −75° C., n-BuLi (73 mL, 182.5 mmol) was dripped into the mixture, the mixture was stirred for 1 h, and a THF (50 mL) solution of Intermediate SM3 (14.8 g, 82.35 mmol) was dripped into the mixture. The reaction temperature was returned to room temperature, and the mixture was stirred for 3 h. After TLC showed that the reaction was completed, the reaction was quenched with an appropriate amount of dilute hydrochloric acid, and layers were separated. The aqueous phase was extracted with DCM. The organic phases were combined, concentrated under reduced pressure and purified through column chromatography to obtain Intermediate B as a solid (24 g, with a yield of 62.7%).

### Step 3: Synthesis of Intermediate C

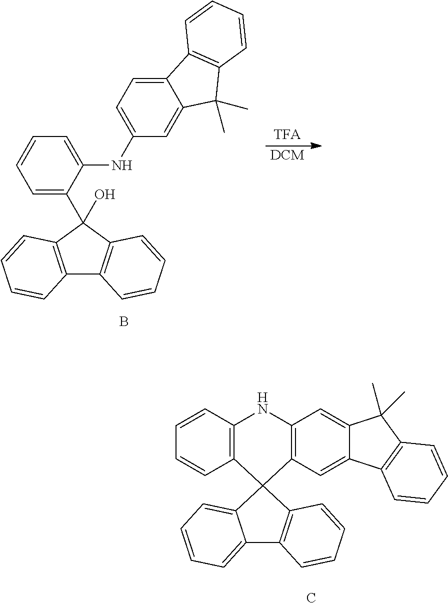

Under an atmosphere of N₂, Intermediate B (24 g, 51.5 mmol) and DCM (300 mL) were added to a 500 mL two-necked flask. TFA (trifluoroacetic acid) (24 mL) was dripped into the mixture at room temperature, and the mixture was stirred overnight at room temperature. After TLC showed that the reaction was completed, the reaction solution was concentrated under reduced pressure and purified through column chromatography to obtain Intermediate C as a solid (21.3 g, with a yield of 92.2%).

### Step 4: Synthesis of Compound A1

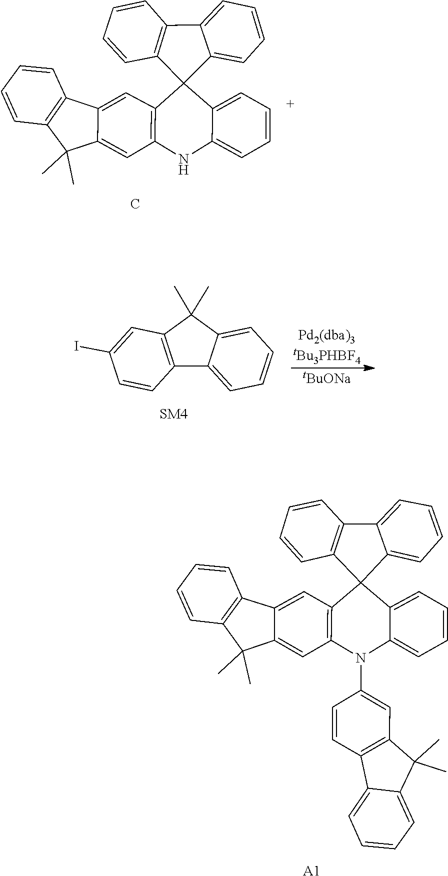

Under an atmosphere of N₂, Intermediate SM4 (716 mg, 2.23 mmol), Intermediate C (1 g, 2.23 mmol), sodium tert-butoxide (430 mg, 4.47 mmol), Pd₂(dba)₃ (41 mg, 0.045 mmol), ^{t}Bu₃PHBF₄ (39 mg, 0.134 mmol) and xylene (10 mL) were added to a three-necked flask in sequence. The temperature was raised to 148° C., and the mixture reacted for 16 h. After TLC showed that the reaction was completed, the reaction temperature was returned to room temperature, and the reaction solution was filtered through Celite, concentrated under reduced pressure and purified through column chromatography to obtain Compound A1 as a white solid (370 mg, with a yield of 26%). The product was confirmed as the target product with a molecular weight of 639.29.

### Synthesis Example 2: Synthesis of Compound B1

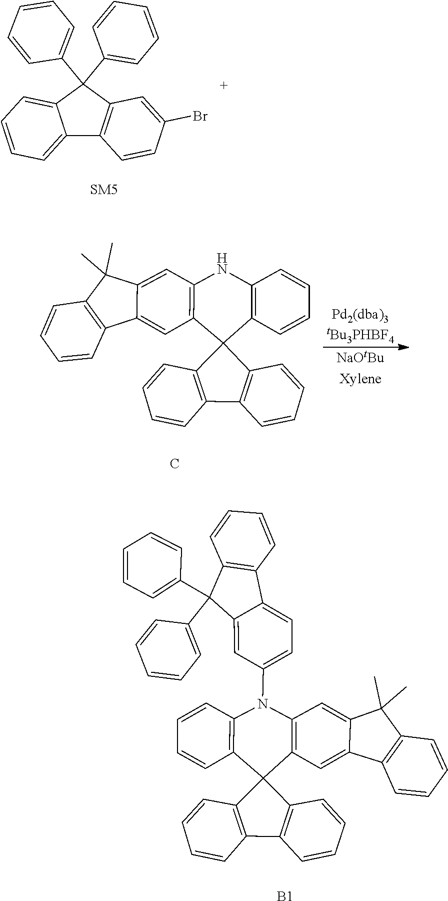

Under an atmosphere of N₂, Intermediate SM5 (0.89 g, 2.23 mmol), Intermediate C (1.0 g, 2.23 mmol), sodium tert-butoxide (430 mg, 4.47 mmol), Pd₂(dba)₃ (41 mg, 0.045 mmol), ^{t}Bu₃PHBF₄ (65 mg, 0.223 mmol) and xylene (40 mL) were added to a 500 mL reaction flask in sequence, and the mixture reacted at 145° C. overnight. After TLC showed that the reaction was completed, the reaction temperature was returned to room temperature, and the reaction solution was filtered through Celite, concentrated under reduced pressure and purified through column chromatography to obtain Compound B1 as a white solid (1.3 g, with a yield of 76.5%). The product was confirmed as the target product with a molecular weight of 763.32.

### Synthesis Example 3: Synthesis of Compound C1

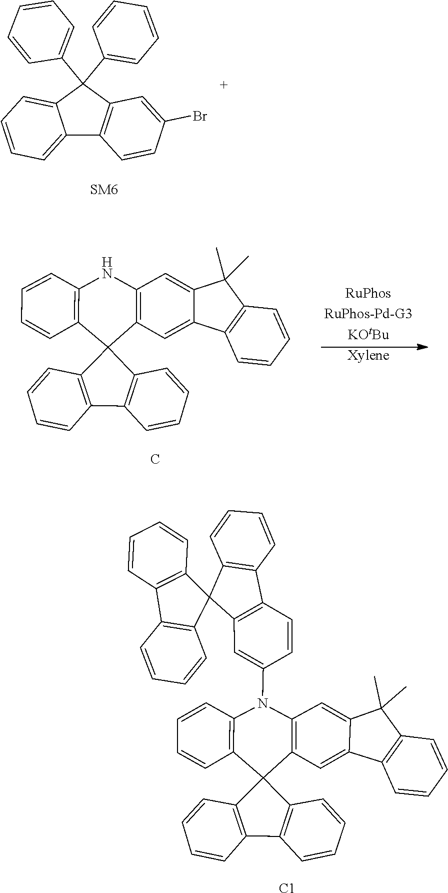

Under an atmosphere of N₂, Intermediate SM6 (0.88 g, 2.23 mmol), Intermediate C (1.0 g, 2.23 mmol), potassium tert-butoxide (0.5 g, 4.46 mmol), Ruphos-Pd-G3 (methanesulfonato(2-dicyclohexylphosphino-2′,6′-diisopropoxy-1,1′-biphenyl)(2-amino-1,1′-biphen yl-2-yl)palladium(II), 28 mg, 0.045 mmol), RuPhos (2-dicyclohexylphosphino-2′,6′-diisopropoxy-1,1′-biphenyl, 21 mg, 0.045 mmol) and xylene (40 mL) were added to a 500 mL reaction flask in sequence, and the mixture reacted at 145° C. overnight. After TLC showed that the reaction was completed, the reaction temperature was returned to room temperature, and the reaction solution was filtered through Celite, concentrated under reduced pressure and purified through column chromatography to obtain Compound C1 as a white solid (1.2 g, with a yield of 70.6%). The product was confirmed as the target product with a molecular weight of 761.31.

### Synthesis Example 4: Synthesis of Compound A2

### Step 1: Synthesis of Intermediate D

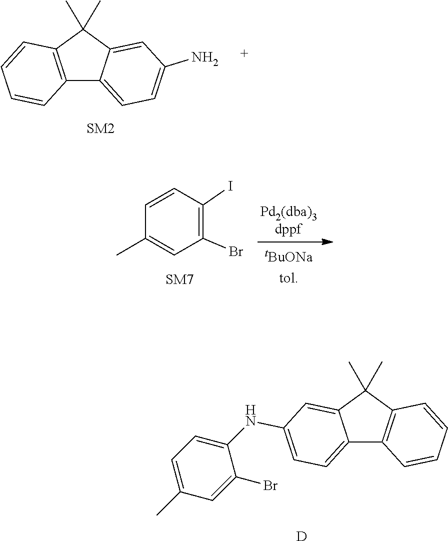

Under an atmosphere of N₂, Intermediate SM2 (14.07 g, 67.3 mmol), Intermediate SM7 (20 g, 67.3 mmol), sodium tert-butoxide (13.1 g, 136.4 mmol), Pd₂(dba)₃ (1.23 g, 1.35 mmol), dppf (3.73 g, 6.73 mmol) and toluene (673 mL) were added to a 2000 mL reaction flask in sequence. The temperature was raised to 120° C., and the mixture reacted for 3 h. After TLC showed that the reaction was completed, the temperature was cooled to room temperature, and the reaction solution was filtered through Celite, concentrated under reduced pressure and purified through column chromatography to obtain Intermediate D as a white solid (16.5 g, with a yield of 65%).

### Step 2: Synthesis of Intermediate E

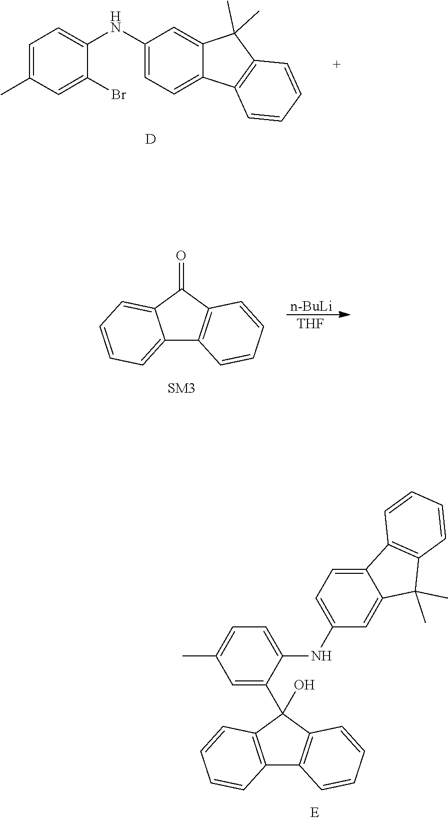

Under an atmosphere of N₂, Intermediate D (16.5 g, 43.6 mmol) and THF (130 mL) were added to a 500 mL two-necked flask. After the temperature was lowered to −82° C., n-BuLi (37 mL, 92.5 mmol) was dripped into the mixture, the mixture was stirred for 1 h, and a THF (20 mL) solution of Intermediate SM3 (9.4 g, 52.3 mmol) was dripped into the mixture. The reaction temperature was returned to room temperature, and the mixture was stirred for 3 h. After TLC showed that the reaction was completed, the reaction was quenched with an appropriate amount of dilute hydrochloric acid, and layers were separated. The aqueous phase was extracted with DCM. The organic phases were combined, concentrated under reduced pressure and purified through column chromatography to obtain Intermediate E as a solid (10.7 g, with a yield of 48.4%).

### Step 3: Synthesis of Intermediate F

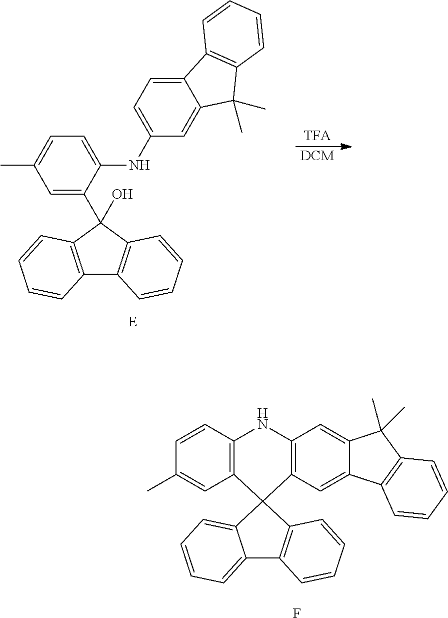

Under an atmosphere of N₂, Intermediate E (10.7 g, 22.3 mmol) and DCM (50 mL) were added to a 100 mL two-necked flask. TFA (11 mL) was dripped into the mixture at room temperature, and the mixture was stirred overnight at room temperature. After TLC showed that the reaction was completed, the reaction solution was concentrated under reduced pressure and purified through column chromatography to obtain Intermediate F as a solid (10.3 g, with a yield of 100%).

### Step 4: Synthesis of Compound A2

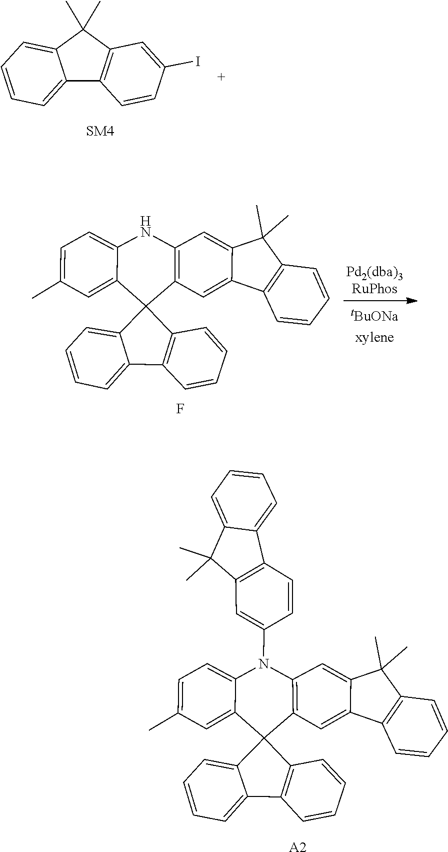

Under an atmosphere of N₂, Intermediate SM4 (1.5 g, 4.688 mmol), Intermediate F (1.79 g, 3.125 mmol), sodium tert-butoxide (600 mg, 6.25 mmol), Pd₂(dba)₃ (0.0625 mmol, 57 mg), Ruphos (0.1875 mmol, 88 mg) and xylene (20 mL) were added to a 250 mL reaction flask in sequence. The temperature was raised to 148° C., and the mixture was reacted for 16 h. After TLC showed that the reaction was completed, the reaction temperature was returned to room temperature, and the reaction solution was filtered through Celite, concentrated under reduced pressure and purified through column chromatography to obtain Compound A2 as a white solid (1 g, with a yield of 49%). The product was confirmed as the target product with a molecular weight of 653.31.

### Synthesis Example 5: Synthesis of Compound B3

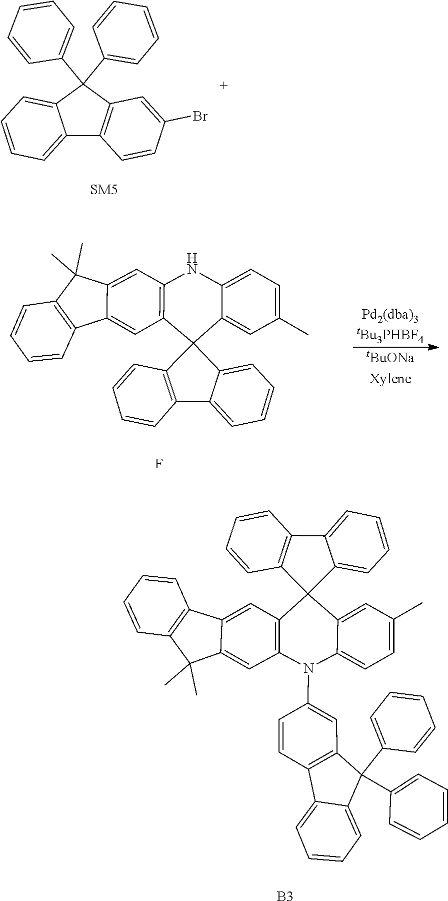

Under an atmosphere of N₂, Intermediate SM5 (693 mg, 1.75 mmol), Intermediate F (1 g, 1.75 mmol), sodium tert-butoxide (336 mg, 3.5 mmol), Pd₂(dba)₃ (32 mg, 0.0353 mmol), ^{t}Bu₃PHBF₄ (31 mg, 0.105 mmol) and xylene (20 mL) were added to a three-necked flask in sequence. The temperature was raised to 148° C., and the mixture was reacted for 16 h. After TLC showed that the reaction was completed, the reaction temperature was returned to room temperature, and the reaction solution was filtered through Celite, concentrated under reduced pressure and purified through column chromatography to obtain Compound B3 as a white solid (1.1 g, with a yield of 81%). The product was confirmed as the target product with a molecular weight of 777.34.

### Synthesis Example 6: Synthesis of Compound B5

### Step 1: Synthesis of Intermediate G

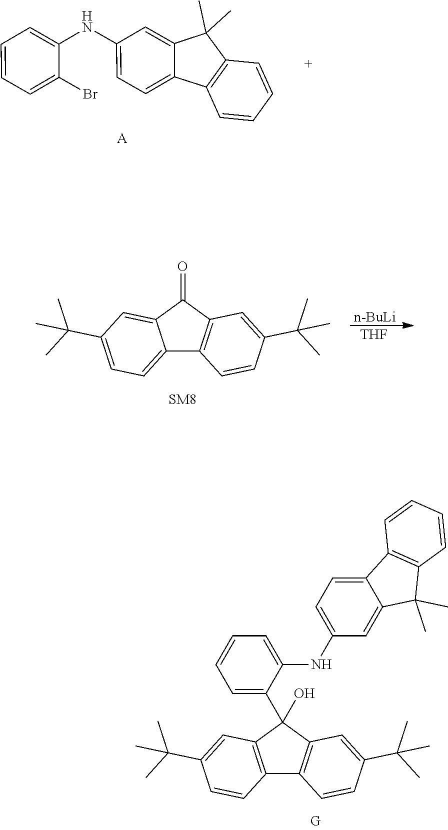

Under an atmosphere of N₂, Intermediate A (10 g, 27.4 mmol) and THF (80 mL) were added to a 500 mL two-necked flask. After the temperature was lowered to −80° C., n-BuLi (23 mL, 57.5 mmol) was dripped into the mixture, the mixture was stirred for 1 h, and a THF (10 mL) solution of Intermediate SM8 (9.6 g, 32.9 mmol) was dripped into the mixture. The reaction temperature was returned to room temperature, and the mixture was stirred for 3 h. After TLC showed that the reaction was completed, the reaction was quenched with an appropriate amount of dilute hydrochloric acid, and layers were separated. The aqueous phase was extracted with DCM. The organic phases were combined, concentrated under reduced pressure and purified through column chromatography to obtain Intermediate G as a solid (12.6 g, with a yield of 79.7%).

### Step 2: Synthesis of Intermediate H

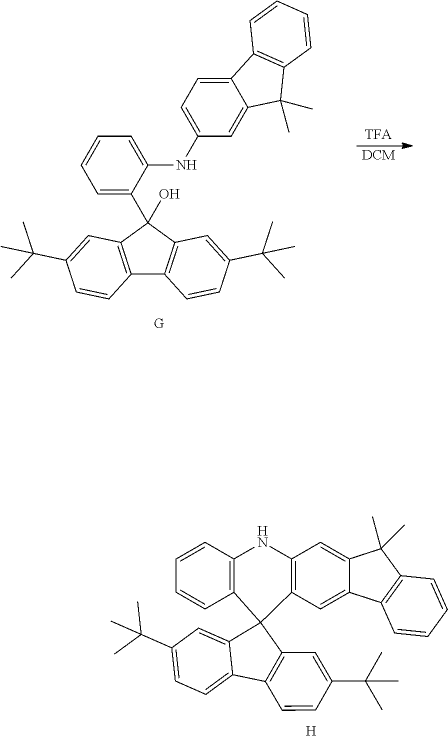

Under an atmosphere of N₂, Intermediate G (12.6 g, 21.8 mmol) and DCM (120 mL) were added to a 200 mL two-necked flask. TFA (13 mL) was dripped into the mixture at room temperature, and the mixture was stirred overnight at room temperature. After TLC showed that the reaction was completed, the reaction solution was concentrated under reduced pressure and purified through column chromatography to obtain Intermediate H as a solid (11 g, with a yield of 91.67%).

### Step 3: Synthesis of Compound B5

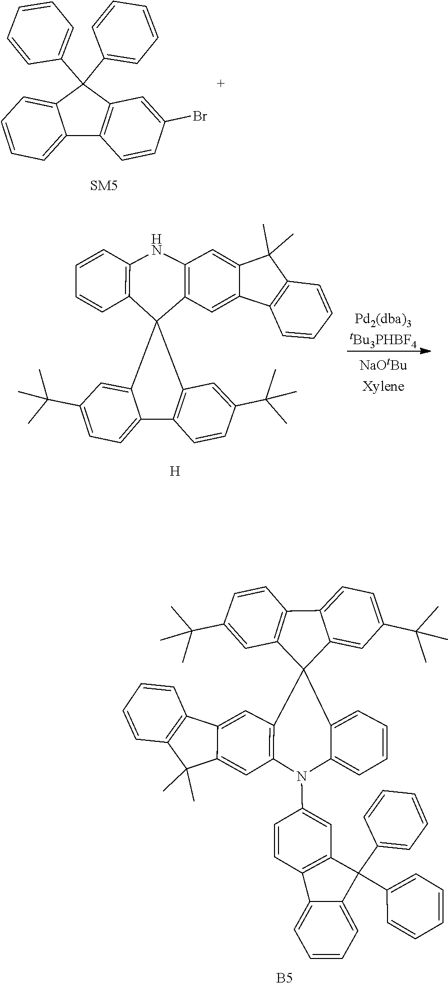

Under an atmosphere of N₂, Intermediate SM5 (0.72 g, 1.82 mmol), Intermediate H (1.0 g, 1.82 mmol), sodium tert-butoxide (430 mg, 4.47 mmol), Pd₂(dba)₃ (41 mg, 0.045 mmol), ^{t}Bu₃PHBF₄ (0.223 mmol, 65 mg) and xylene (40 mL) were added to a 500 mL reaction flask in sequence, and the mixture reacted at 145° C. overnight. After TLC showed that the reaction was completed, the reaction temperature was returned to room temperature, and the reaction solution was filtered through Celite, concentrated under reduced pressure and purified through column chromatography to obtain Compound B5 as a white solid (1.2 g, with a yield of 75.9%). The product was confirmed as the target product with a molecular weight of 875.45.

### Synthesis Example 7: Synthesis of Compound A18

### Step 1: Synthesis of Intermediate I

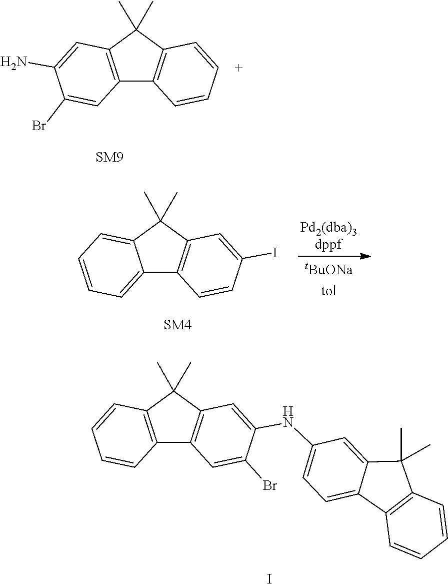

Under an atmosphere of N₂, Intermediate SM9 (17.9 g, 62.5 mmol), Intermediate SM4 (20 g, 67.3 mmol), sodium tert-butoxide (12 g, 125 mmol), Pd₂(dba)₃ (1.15 g, 1.25 mmol), dppf (2.1 g, 3.75 mmol) and toluene (700 mL) were added to a 2000 mL reaction flask in sequence. The temperature was raised to 120° C., and the mixture reacted for 3 h. After TLC showed that the reaction was completed, the temperature was cooled to room temperature, and the reaction solution was filtered through Celite, concentrated under reduced pressure and purified through column chromatography to obtain Intermediate I as a white solid (22 g, with a yield of 73.6%).

### Step 2: Synthesis of Intermediate J

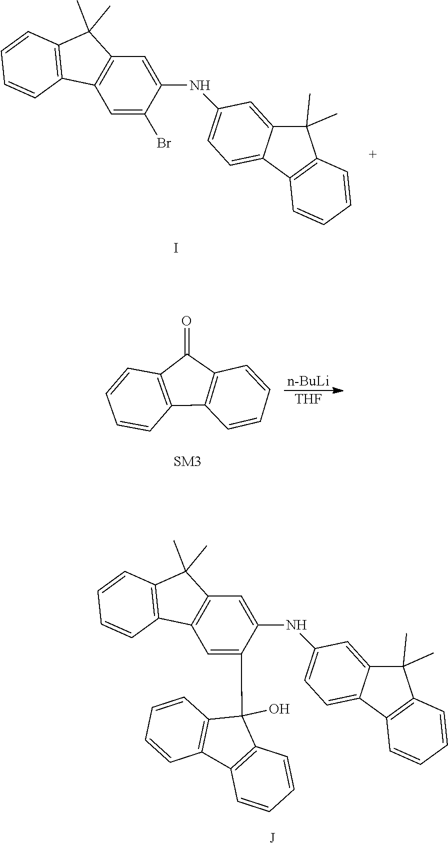

Under an atmosphere of N₂, Intermediate I (22 g, 45.9 mmol) and THF (200 mL) were added to a 500 mL three-necked flask. After the temperature was lowered to −90° C., n-BuLi (39 mL, 97.5 mmol) was dripped into the mixture, the temperature was raised to −80° C., the mixture was stirred for 40 min, and a THF (20 mL) solution of Intermediate SM3 (8.3 g, 45.9 mmol) was dripped into the mixture. The reaction temperature was returned to room temperature. After TLC showed that the reaction was completed, the reaction was quenched with an appropriate amount of dilute hydrochloric acid, and layers were separated. The aqueous phase was extracted with DCM. The organic phases were combined, concentrated under reduced pressure and purified through column chromatography to obtain Intermediate J as a solid (17 g, with a yield of 63.7%).

### Step 3: Synthesis of Intermediate K

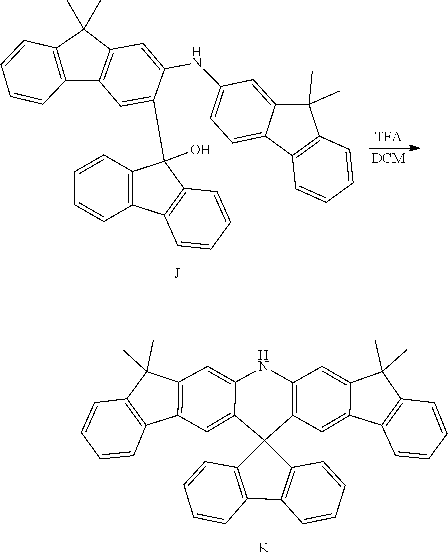

Under an atmosphere of N₂, Intermediate J (17 g, 29.3 mmol) and DCM (500 mL) were added to a 500 mL single-necked flask. TFA (20 mL) was dripped into the mixture at room temperature, and the mixture was stirred overnight at room temperature. After TLC showed that the reaction was completed, the reaction solution was concentrated under reduced pressure and purified through column chromatography to obtain Intermediate K as a solid (13.4 g, with a yield of 81.2%).

### Step 4: Synthesis of Compound A18

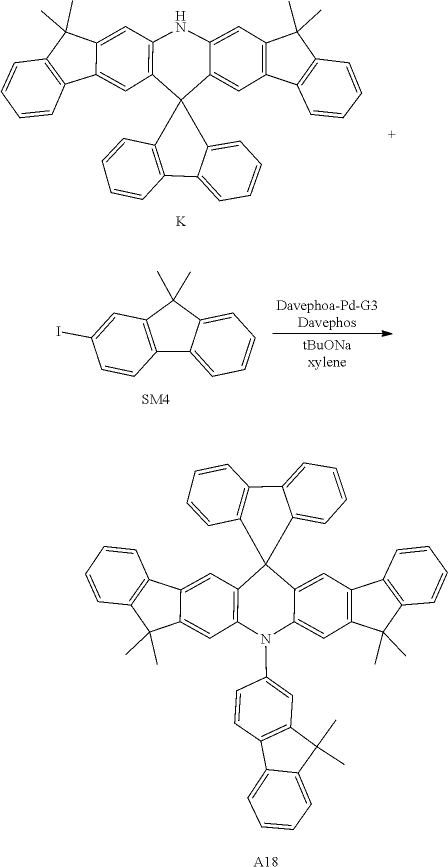

Under an atmosphere of N₂, Intermediate SM4 (1.1 g, 3.45 mmol), Intermediate K (1.3 g, 2.3 mmol), sodium tert-butoxide (441 mg, 4.6 mmol), DavePhos-Pd-G3(methanesulfonato[2-(dicyclohexylphosphino)-2′-(N,N-dimethylamino)-1,1′-biphenyl](2′-amino-1,1′-biphenyl-2-yl)palladium(II), 35 mg, 0.046 mmol), DavePhos (2-dicyclohexylphosphino-2′-(N,N-dimethylamino)biphenyl, 54 mg, 0.138 mmol) and xylene (20 mL) were added to a three-necked flask in sequence. The temperature was raised to 148° C., and the mixture was reacted for 16 h. After TLC showed that the reaction was completed, the reaction temperature was returned to room temperature, and the reaction solution was filtered through Celite, concentrated under reduced pressure and purified through column chromatography to obtain Compound A18 as a white solid (1.22 g, with a yield of 70%). The product was confirmed as the target product with a molecular weight of 755.36.

Those skilled in the art will appreciate that the above preparation methods are merely exemplary. Those skilled in the art can obtain other compound structures of the present disclosure through the modifications of the preparation methods.

The method for preparing an organic electroluminescent device is not limited. The preparation methods in the following device examples are merely examples and are not to be construed as limitations. Those skilled in the art can make reasonable improvements to the preparation methods in the following device examples based on the related art.

### DEVICE EXAMPLE

### Device Example 1: Preparation of an Organic Electroluminescent Device

Firstly, a glass substrate having a thickness of 0.7 mm and patterned with an indium tin oxide (ITO) anode with a thickness of 800 Å was washed with deionized water and a detergent, and then the ITO surface was treated with oxygen plasma and UV ozone. The substrate was dried in a glovebox to remove moisture, mounted on a substrate holder, and transferred into a vacuum chamber. Organic layers specified below were sequentially deposited on the anode layer through vacuum thermal evaporation at a rate of 0.01-10 Å/s and at a vacuum degree of about 10⁻⁶ Torr. Compound HT and Compound HI were co-deposited to form a hole injection layer (HIL, with a weight ratio of 97:3, 100 Å). Compound HT was deposited to form a hole transport layer (HTL, 1100 Å). Compound A1 of the present disclosure was deposited to form an electron blocking layer (EBL, 550 Å). Compound H-1, Compound H-2 and Compound GD were co-deposited to form a light-emitting layer (EML, with a weight ratio of 47:47:6, 400 Å). Compound HB was deposited to form a hole blocking layer (HBL, 50 Å). Compound ET and Liq were co-deposited to form an electron transport layer (ETL, 40:60, 350 Å). Liq with a thickness of 10 Å was deposited to form an electron injection layer (EIL). Finally, the metal aluminum was deposited to form a cathode (1200 Å). The device was transferred back to the glovebox and encapsulated with a glass lid to complete the device.

### Device Example 2

The preparation method in Device Example 2 was the same as the preparation method in Device Example 1, except that in the electron blocking layer, Compound A1 of the present disclosure was replaced with Compound A18 of the present disclosure.

### Device Example 3

The preparation method in Device Example 3 was the same as the preparation method in Device Example 1, except that in the electron blocking layer, Compound A1 of the present disclosure was replaced with Compound A2 of the present disclosure.

### Device Example 4

The preparation method in Device Example 4 was the same as the preparation method in Device Example 1, except that in the electron blocking layer, Compound A1 of the present disclosure was replaced with Compound B1 of the present disclosure.

### Device Example 5

The preparation method in Device Example 5 was the same as the preparation method in Device Example 1, except that in the electron blocking layer, Compound A1 of the present disclosure was replaced with Compound B3 of the present disclosure.

### Device Example 6

The preparation method in Device Example 6 was the same as the preparation method in Device Example 1, except that in the electron blocking layer, Compound A1 of the present disclosure was replaced with Compound B5 of the present disclosure.

### Device Example 7

The preparation method in Device Example 7 was the same as the preparation method in Device Example 1, except that in the electron blocking layer, Compound A1 of the present disclosure was replaced with Compound C1 of the present disclosure.

### Device Comparative Example 1

The preparation method in Device Comparative Example 1 was the same as the preparation method in Device Example 1, except that in the electron blocking layer, Compound A1 of the present disclosure was replaced with Compound EB-1.

### Device Comparative Example 2

The preparation method in Device Comparative Example 2 was the same as the preparation method in Device Example 1, except that in the electron blocking layer, Compound A1 of the present disclosure was replaced with Compound EB-2.

### Device Comparative Example 3

The preparation method in Device Comparative Example 3 was the same as the preparation method in Device Example 1, except that in the electron blocking layer, Compound A1 of the present disclosure was replaced with Compound EB-3.

Detailed structures and thicknesses of layers of the devices are shown in Table 1. A layer using more than one material is obtained by doping different compounds at their weight ratio as recorded.

TABLE 1
Part of device structures in Examples 1 to 7 and Comparative Examples 1 to 3

| Device No. | HIL | HTL | EBL | EML | HBL | ETL |
|---|---|---|---|---|---|---|
| Example 1 | Compound | Compound | Compound | Compound | Compound | Compound |
| | HT:Compound | HT | A1 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 2 | Compound | Compound | Compound | Compound | Compound | Compound |
| | HT:Compound | HT | A18 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 3 | Compound | Compound | Compound | Compound | Compound | Compound |
| | HT:Compound | HT | A2 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 4 | Compound | Compound | Compound | Compound | Compound | Compound |
| | HT:Compound | HT | B1 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 5 | Compound | Compound | Compound | Compound | Compound | Compound |
| | HT:Compound | HT | B3 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 6 | Compound | Compound | Compound | Compound | Compound | Compound |
| | HT:Compound | HT | B5 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Example 7 | Compound | Compound | Compound | Compound | Compound | Compound |
| | HT:Compound | HT | C1 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Comparative | Compound | Compound | Compound | Compound | Compound | Compound |
| Example 1 | HT:Compound | HT | EB-1 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Comparative | Compound | Compound | Compound | Compound | Compound | Compound |
| Example 2 | HT:Compound | HT | EB-2 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |
| Comparative | Compound | Compound | Compound | Compound | Compound | Compound |
| Example 3 | HT:Compound | HT | EB-3 | H-1:Compound | HB | ET:Liq |
| | HI (97:3) | (1100 Å) | (550 Å) | H-2:Compound | (50 Å) | (40:60) |
| | (100 Å) | | | GD (47:47:6) | | (350 Å) |
| | | | | (400 Å) | | |

The materials use in the devices have the following structures:

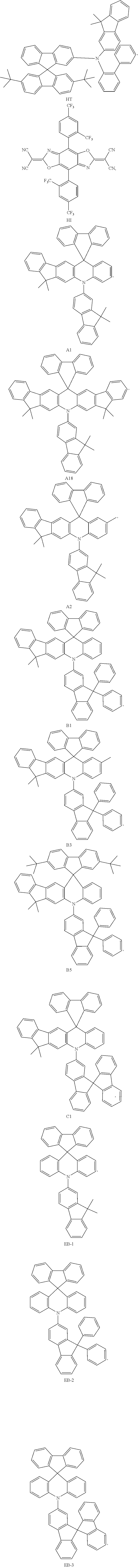

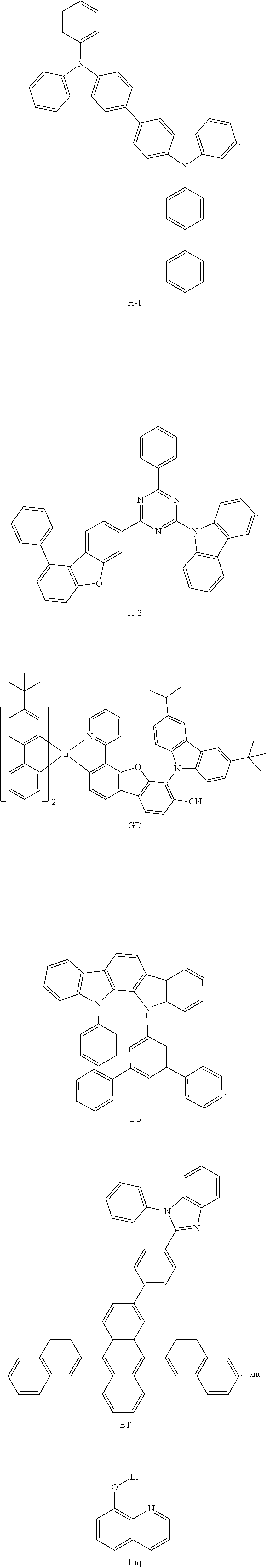

The CIE, voltage and power efficiency (PE) of Examples 1 to 3 and Comparative Example 1 were measured at a current density of 10 mA/cm², and the lifetimes (LT95) of Examples 1 to 3 and Comparative Example 1 were measured at a current density of 80 mA/cm². For a more intuitive comparison, the LT95 of Comparative Example 1 was set to 1, and the LT95 of Examples 1 to 3 was converted relative to the LT95 of Comparative Example 1. The data are recorded and shown in Table 2.

TABLE 2
Device data of Examples 1 to 3 and Comparative Example 1

| | | Voltage | Power efficiency | |
|---|---|---|---|---|
| Device No. | CIE (x, y) | (V) | (lm/W) | LT95 |
| Example 1 | (0.350, 0.625) | 3.6 | 74 | 50 |
| Example 2 | (0.350, 0.625) | 3.5 | 69 | 50 |
| Example 3 | (0.346, 0.628) | 3.4 | 76 | 50 |
| Comparative | (0.349, 0.626) | 3.9 | 68 | 1 |
| Example 1 | | | | |

### Discussion:

As can be seen from the data in Table 2, the CIE values of Examples 1 to 3 and Comparative Example 1 basically remain consistent.

Compounds A1, A18 and A2 of the present disclosure used in Examples 1 to 3 and Compound EB-1 used in Comparative Example 1 all possess a structure of fluorene-spiroacridine joined to 9,9-dimethylfluorene. The primary distinction lies in the presence of an additional specific fused structure on the acridine ring in the compounds of the present disclosure, yet the resulting device performance differences are remarkably significant. As can be seen from the data in Table 2, compared to Comparative Example 1, the voltages of Examples 1 to 3 are reduced by 0.3 V, 0.4 V, and 0.5 V, respectively; the power efficiencies of Examples 1 to 3 are significantly improved by 9%, 1.5%, and 12%, respectively; and, more importantly, the lifetimes of Examples 1 to 3 are unexpectedly and substantially enhanced by 49 times. The above data indicates that due to the specific fused acridine-spirofluorene structure, the compounds of the present disclosure can significantly reduce the voltage, further improve the device efficiency, and particularly, substantially enhance the device lifetime, thereby providing superior overall device performance.

The CIE, voltage and PE of Examples 4 to 6 and Comparative Example 2 were measured at a current density of 10 mA/cm², and the LT95 of Examples 4 to 6 and Comparative Example 2 was measured at a current density of 80 mA/cm². For a more intuitive comparison, the LT95 of Comparative Example 2 was set to 1, and the LT95 of Examples 4 to 6 was converted relative to the LT95 of Comparative Example 2. The data are recorded and shown in Table 3.

TABLE 3
Device data of Examples 4 to 6 and Comparative Example 2

| | | Voltage | Power efficiency | |
|---|---|---|---|---|
| Device No. | CIE (x, y) | (V) | (lm/W) | LT95 |
| Example 4 | (0.351, 0.624) | 3.4 | 73 | 8.7 |
| Example 5 | (0.350, 0.625) | 4.1 | 59 | 6.3 |
| Example 6 | (0.351, 0.625) | 5.1 | 49 | 4.3 |
| Comparative | (0.346, 0.628) | 4.6 | 57 | 1 |
| Example 2 | | | | |

### Discussion:

As can be seen from the data in Table 3, the CIE values of Examples 4 to 6 and Comparative Example 2 basically remain consistent.

Compounds B1, B3 and B5 of the present disclosure used in Examples 4 to 6 and Compound EB-2 used in Comparative Example 2 all possess a structure of fluorene-spiroacridine joined to 9,9-dimethylfluorene. The primary distinction lies in the presence of an additional specific fused structure on the acridine ring in the compounds of the present disclosure, yet the resulting device performance differences were remarkably significant. As can be seen from the data in Table 3, compared to Comparative Example 2, the voltages of Examples 4 to 6 are maintained at low levels or significantly reduced, and the power efficiencies of Examples 4 to 6 are maintained at high levels or significantly improved. In particular, Example 4 shows a particularly notable 28% increase in power efficiency. More importantly, compared to Comparative Example 2, Examples 4 to 6 exhibit unexpectedly and substantially enhanced lifetimes by 7.7 times, 5.3 times, and 3.3 times, respectively. The above data further indicates that due to the specific fused acridine-spirofluorene structure, the compounds of the present disclosure can provide superior overall device performance and particularly, substantially enhance the device lifetime.

The CIE, voltage and PE of Example 7 and Comparative Example 3 were measured at a current density of 10 mA/cm², and the LT95 of Example 7 and Comparative Example 3 was measured at a current density of 80 mA/cm². For a more intuitive comparison, the LT95 of Comparative Example 3 was set to 1, and the LT95 of Example 7 was converted relative to the LT95 of Comparative Example 3. The data are recorded and shown in Table 4.

TABLE 4
Device data of Example 7 and Comparative Example 3

| | | Voltage | Power efficiency | |
|---|---|---|---|---|
| Device No. | CIE (x, y) | (V) | (lm/W) | LT95 |
| Example 7 | (0.347, 0.628) | 3.4 | 75 | 2.8 |
| Comparative | (0.346, 0.629) | 4.4 | 60 | 1 |
| Example 3 | | | | |

As can be seen from the data in Table 4, the CIE values of Example 7 and Comparative Example 3 basically remain consistent.

Compound C1 of the present disclosure used in Example 7 and Compound EB-3 used in Comparative Example 3 both possess a structure of fluorene-spiroacridine joined to spirofluorene. The primary distinction lies in the presence of an additional specific fused structure on the acridine ring in the compound of the present disclosure, yet the resulting device performance differences are remarkably significant. As can be seen from the data in Table 4, compared to Comparative Example 3, the voltage of Example 7 is significantly reduced by 1.0 V, the power efficiency of Example 7 is significantly improved by 25%, and, more importantly, the lifetime of Example is unexpectedly and substantially enhanced by 1.8 times. The above data further indicates that due to the specific fused acridine-spirofluorene structure, the compounds of the present disclosure can significantly reduce the voltage, further improve the efficiency, and particularly, substantially enhance the lifetime, thereby providing superior overall device performance.

In conclusion, when applied in organic electroluminescent devices, the compound represented by Formula 1 and having a specific structure in the present disclosure can maintain a low voltage or significantly reduce the voltage, maintain high efficiency or significantly improve efficiency, and particularly, dramatically enhance the device lifetime, thereby providing superior overall device performance and exhibiting very broad application prospects.

It should be understood that various embodiments described herein are merely embodiments and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It should be understood that various theories as to why the present disclosure works are not intended to be limitative.

What is claimed is:

1. A compound, having a structure represented by Formula 1: 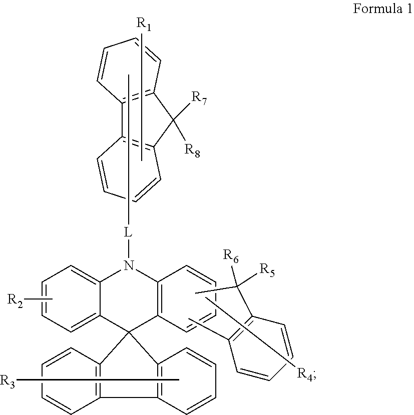
wherein
L is selected from a single bond, substituted or unsubstituted arylene having 6 to 30 carbon atoms, substituted or unsubstituted heteroarylene having 3 to 30 carbon atoms or a combination thereof;
R₁, R₂, R₃ and R₄ represent, at each occurrence identically or differently, mono-substitution, multiple substitutions or non-substitution;
R₁, R₂, R₃, R₅, R₆, R₇ and R₈ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₇ and R₈ can be optionally joined to form a ring.

2. The compound of claim 1, wherein the compound has a structure represented by Formula 2: 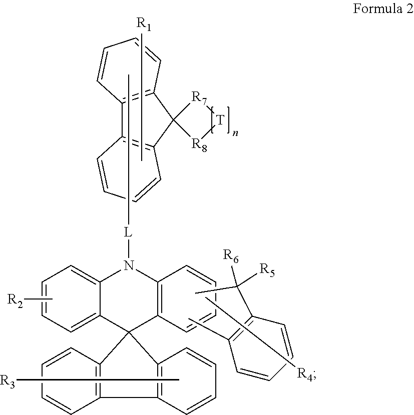
wherein T is selected from a single bond, O, S or CR₉R₁₀;
n is selected from 0 or 1;
L is selected from a single bond, substituted or unsubstituted arylene having 6 to 30 carbon atoms, substituted or unsubstituted heteroarylene having 3 to 30 carbon atoms or a combination thereof;
R₁, R₂, R₃ and R₄ represent, at each occurrence identically or differently, mono-substitution, multiple substitutions or non-substitution;
R₁, R₂, R₃, R₅, R₆, R₇, R₈, R₉ and R₁₀ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁, R₂, R₃, R₄, R₅, R₆, R₉ and R₁₀ can be optionally joined to form a ring.

3. The compound of claim 1, wherein at least two adjacent substituents R₂ are joined to form a ring; and
preferably, at least two adjacent substituents R₂ are joined to form a substituted or unsubstituted aromatic ring having 6 to 30 carbon atoms or a substituted or unsubstituted heteroaromatic ring having 3 to 30 carbon atoms.

4. The compound of claim 1, wherein the compound has a structure represented by any one of Formula 3-1 to Formula 3-9: 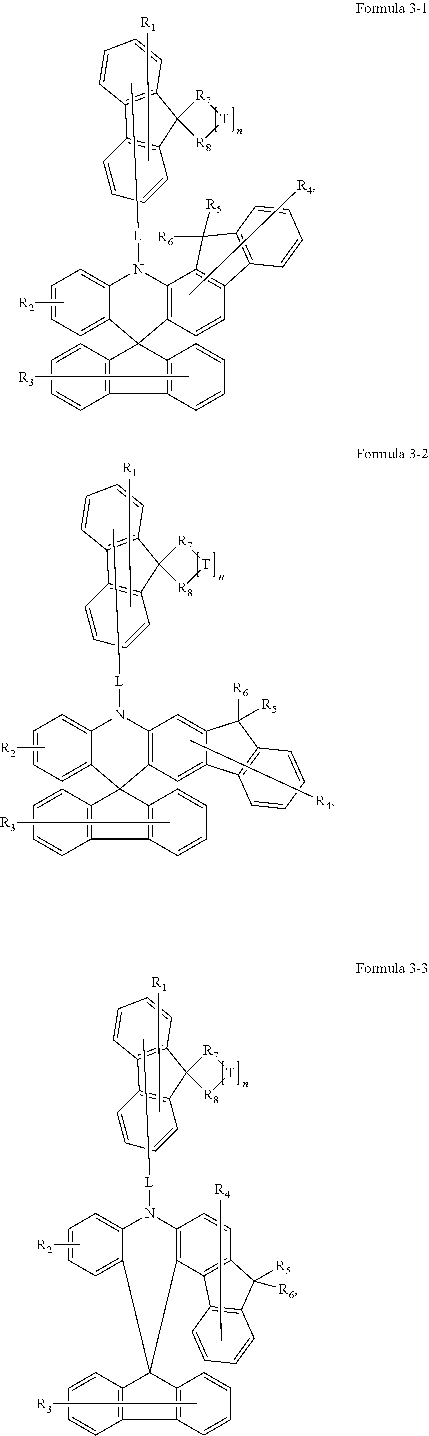 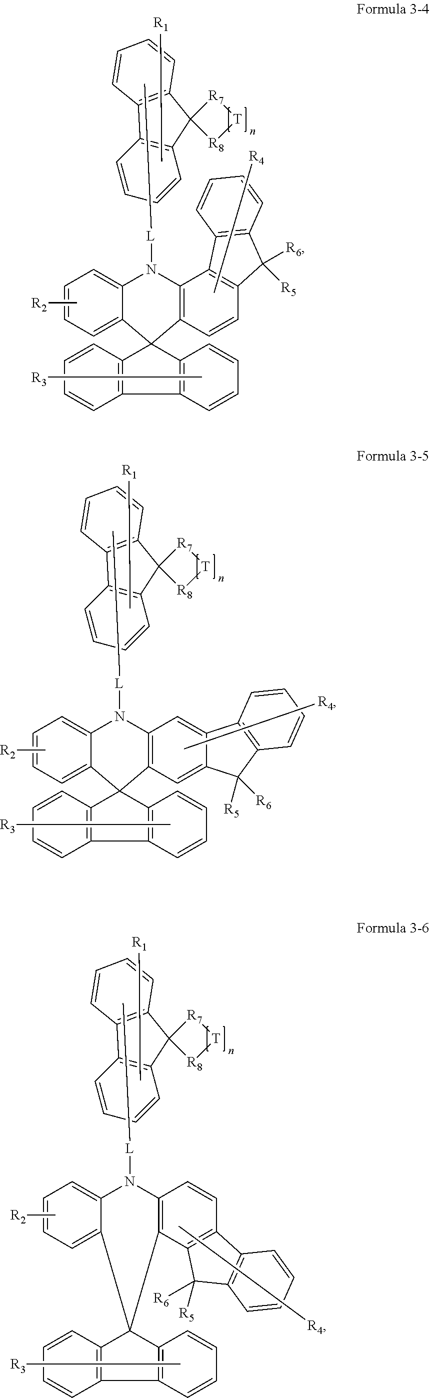 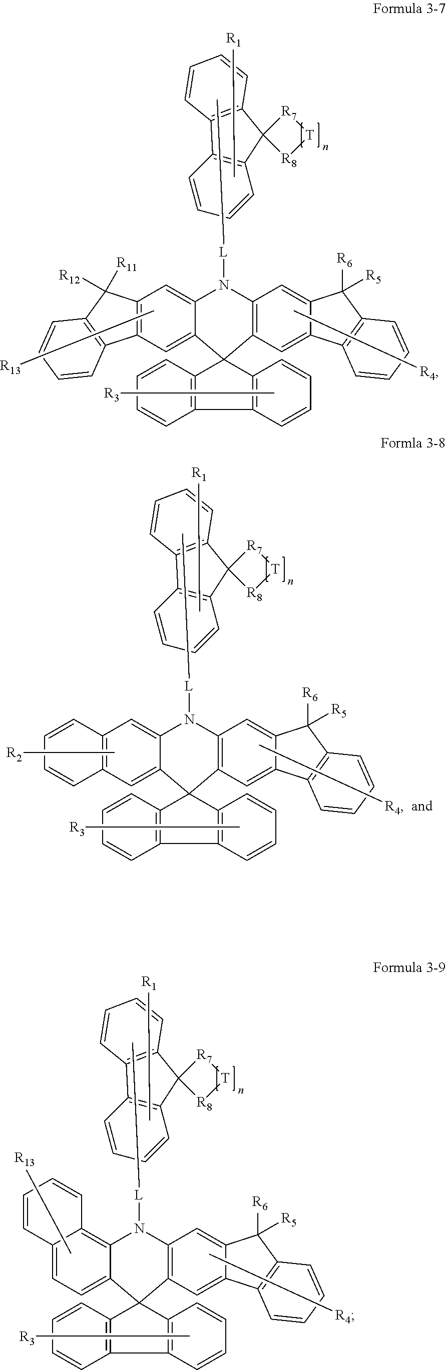
wherein T is selected from a single bond, O, S or CR₉R₁₀;
n is selected from 0 or 1;
R₁, R₂, R₃, R₄ and R₁₃ represent, at each occurrence identically or differently, mono-substitution, multiple substitutions or non-substitution;
R₁, R₂, R₃, R₅, R₆, R₇, R₈, R₉, R₁₀, R₁₁, R₁₂ and R₁₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₁, R₂, R₃, R₄, R₅, R₆, R₉, R₁₀, R₁₁, R₁₂ and R₁₃ can be optionally joined to form a ring; and
preferably, the compound has a structure represented by Formula 3-2, Formula 3-5, Formula 3-7, Formula 3-8 or Formula 3-9.

5. The compound of claim 1, wherein the L is selected from a single bond or substituted or unsubstituted arylene having 6 to 30 carbon atoms;
preferably, the L is selected from a single bond, substituted or unsubstituted phenylene, substituted or unsubstituted biphenylylene, substituted or unsubstituted naphthylene or substituted or unsubstituted fluorenylidene; and
more preferably, the L is selected from a single bond or substituted or unsubstituted phenylene.

6. The compound of claim 4, wherein the R₁, R₂, R₃, R₄, R₅, R₆, R₇, R₈, R₉, R₁₀, R₁₁, R₁₂ and R₁₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof; and
preferably, the R₁, R₂, R₃, R₄, R₅, R₆, R₇, R₈, R₉, R₁₀, R₁₁, R₁₂ and R₁₃ are, at each occurrence identically or differently, selected from the group consisting of: hydrogen, deuterium, fluorine, substituted or unsubstituted methyl, substituted or unsubstituted ethyl, substituted or unsubstituted isopropyl, substituted or unsubstituted tert-butyl, substituted or unsubstituted cyclopentyl, substituted or unsubstituted cyclohexyl, substituted or unsubstituted adamantyl, substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted dibenzofuryl, substituted or unsubstituted dibenzothienyl, substituted or unsubstituted dibenzoselenophenyl, substituted or unsubstituted carbazolyl, substituted or unsubstituted phenanthryl, substituted or unsubstituted anthryl, and combinations thereof.

7. The compound of claim 4, wherein at least one of the R₁, R₂, R₃, R₄ or R₁₃ is, at each occurrence identically or differently, selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof; and
preferably, at least one of the R₁, R₂, R₃, R₄ or R₁₃ is, at each occurrence identically or differently, selected from the group consisting of: deuterium, substituted or unsubstituted methyl, substituted or unsubstituted ethyl, substituted or unsubstituted isopropyl, substituted or unsubstituted tert-butyl, substituted or unsubstituted cyclopentyl, substituted or unsubstituted cyclohexyl, substituted or unsubstituted adamantyl, substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted dibenzofuryl, substituted or unsubstituted dibenzothienyl, substituted or unsubstituted dibenzoselenophenyl, substituted or unsubstituted carbazolyl, substituted or unsubstituted phenanthryl, substituted or unsubstituted anthryl, and combinations thereof.

8. The compound of claim 2, wherein n is selected from 0, and the R₇ and R₈ are, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof;
preferably, n is selected from 0, and the R₇ and R₈ are, at each occurrence identically or differently, selected from the group consisting of: substituted or unsubstituted alkyl having 1 to 20 carbon atoms, substituted or unsubstituted aryl having 6 to 30 carbon atoms, and combinations thereof; and
more preferably, n is selected from 0, and the R₇ and R₈ are, at each occurrence identically or differently, selected from substituted or unsubstituted methyl or substituted or unsubstituted phenyl.

9. The compound of claim 2, wherein n is selected from 1, and T is selected from a single bond or CR₉R₁₀;
preferably, n is selected from 1, T is selected from a single bond, and the R₇ and R₈ are, at each occurrence identically or differently, selected from the group consisting of: substituted or unsubstituted alkylene having 1 to 20 carbon atoms, substituted or unsubstituted cycloalkylene having 3 to 20 ring carbon atoms, substituted or unsubstituted arylene having 6 to 30 carbon atoms, substituted or unsubstituted heteroarylene having 3 to 30 carbon atoms, and combinations thereof; and
more preferably, n is selected from 1, T is selected from a single bond, and the R₇ and R₈ are selected from substituted or unsubstituted phenylene.

10. The compound of claim 1, wherein the compound is selected from the group consisting of Compound A1 to Compound A124, Compound B1 to Compound B92, and Compound C1 to Compound C98: 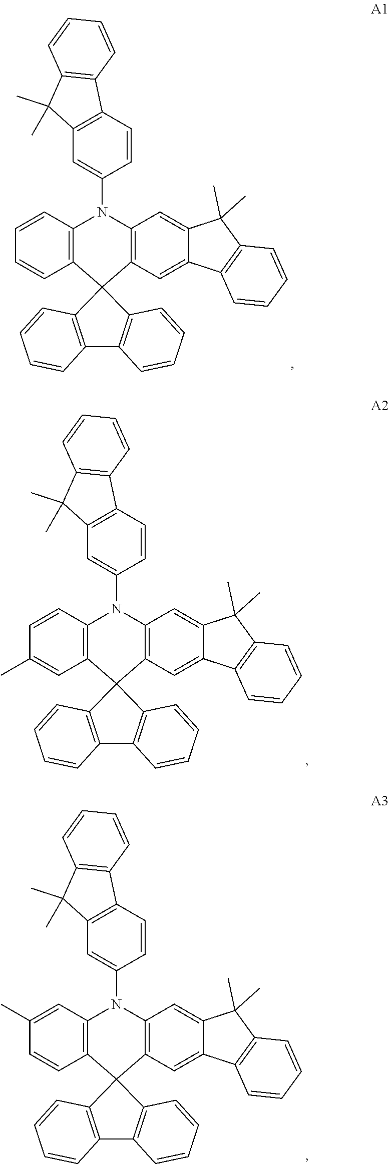 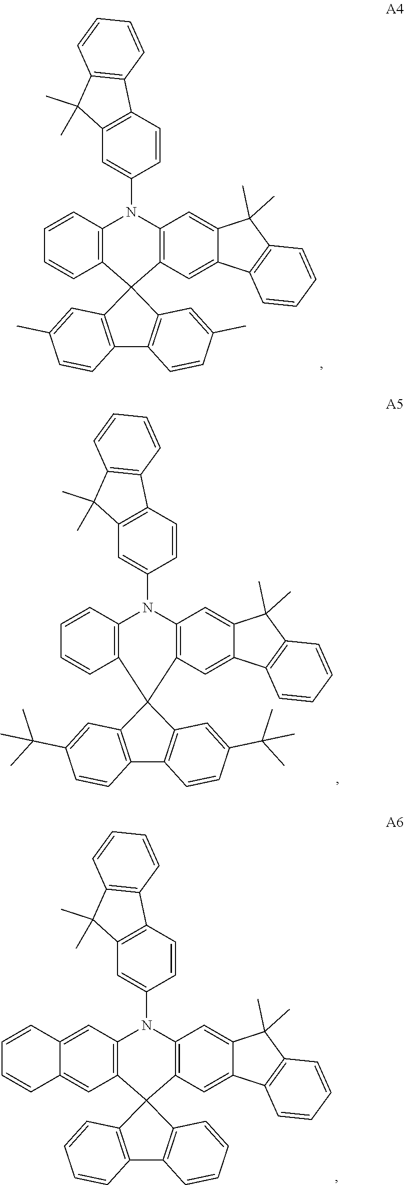 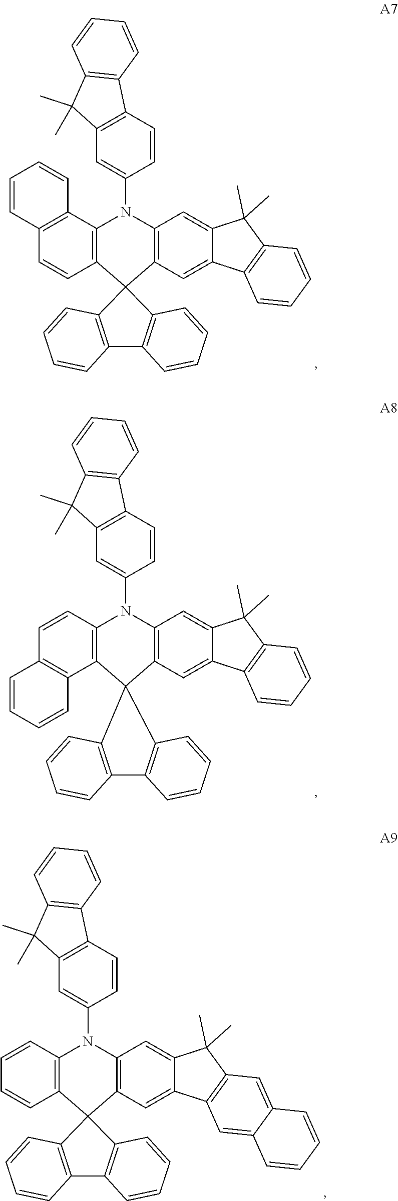 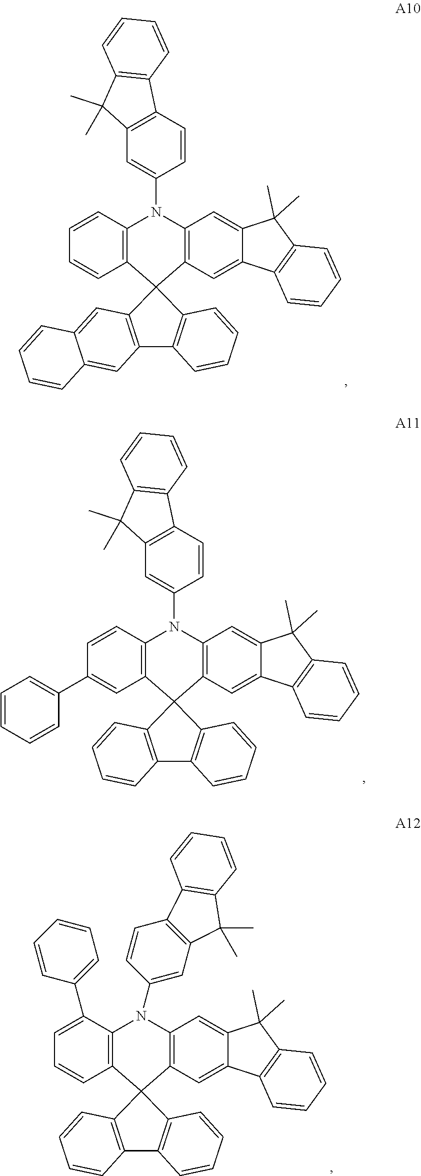 [SMILES: CC1(C)c2ccccc2-c2ccc(N3c4ccccc4C4(c5ccccc5-c5cc6ccccc6cc54)c4cc5c(cc43)C(C)(C)c3ccccc3-5)cc21 ; CC1(C)c2ccccc2-c2ccc(N3c4ccc(-c5ccccc5)cc4C4(c5ccccc5-c5ccccc54)c4cc5c(cc43)C(C)(C)c3ccccc3-5)cc21 ; CC1(C)c2ccccc2-c2ccc(N3c4cc5c(cc4C4(c6ccccc6-c6ccccc64)c4cccc(-c6ccccc6)c43)-c3ccccc3C5(C)C)cc21] 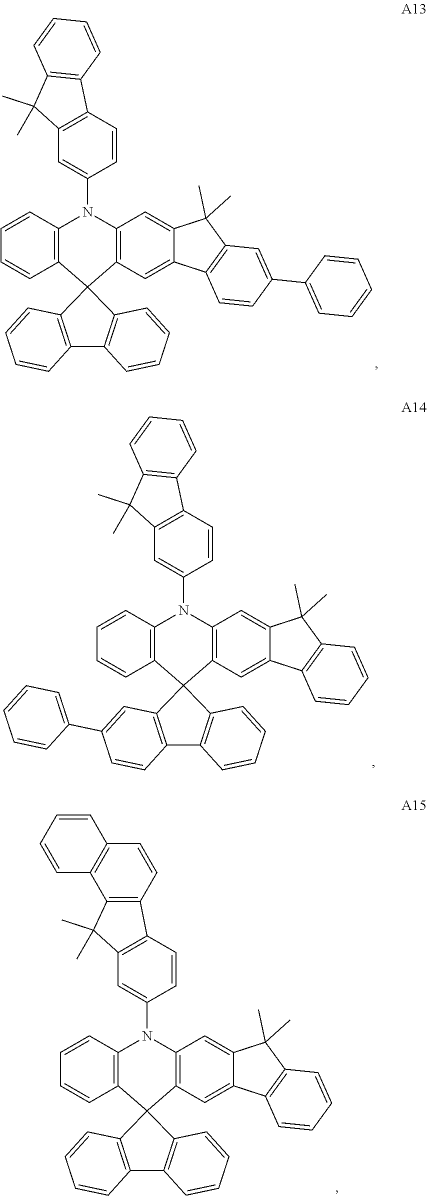 [SMILES: CC1(C)c2ccccc2-c2ccc(N3c4ccccc4C4(c5ccccc5-c5ccccc54)c4cc5c(cc43)C(C)(C)c3cc(-c4ccccc4)ccc3-5)cc21 ; CC1(C)c2ccccc2-c2ccc(N3c4ccccc4C4(c5ccccc5-c5ccc(-c6ccccc6)cc54)c4cc5c(cc43)C(C)(C)c3ccccc3-5)cc21 ; CC1(C)c2ccccc2-c2cc3c(cc21)N(c1ccc2c(c1)C(C)(C)c1c-2ccc2ccccc12)c1ccccc1C31c2ccccc2-c2ccccc21] 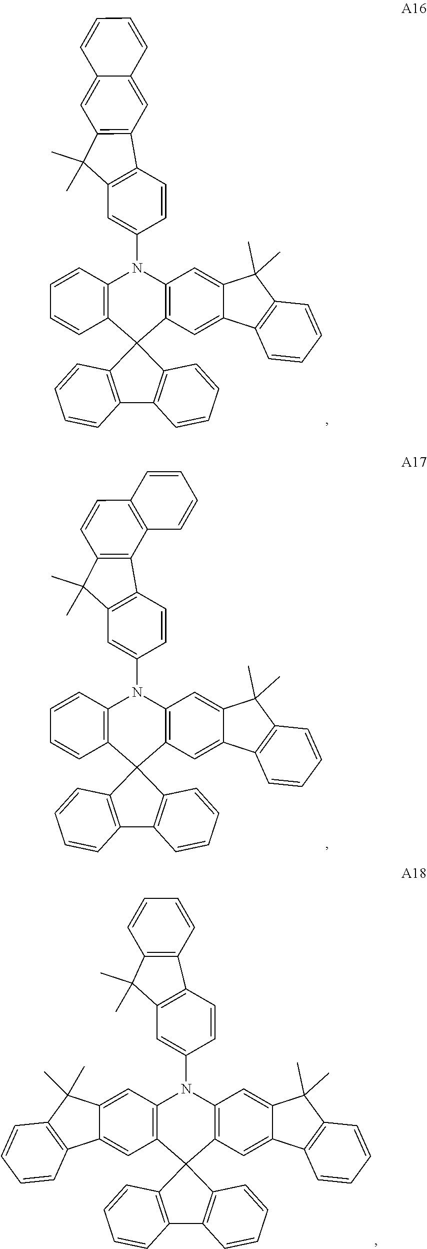 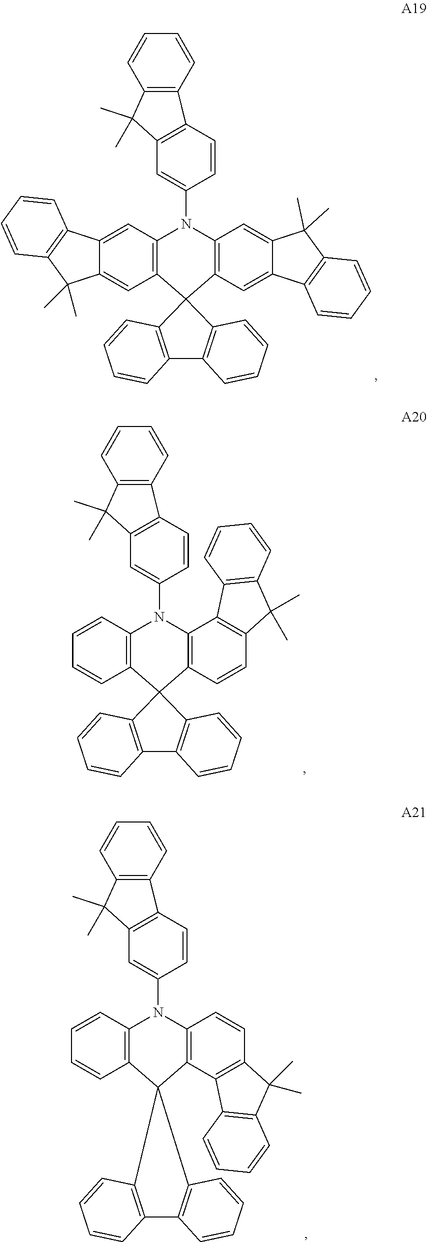 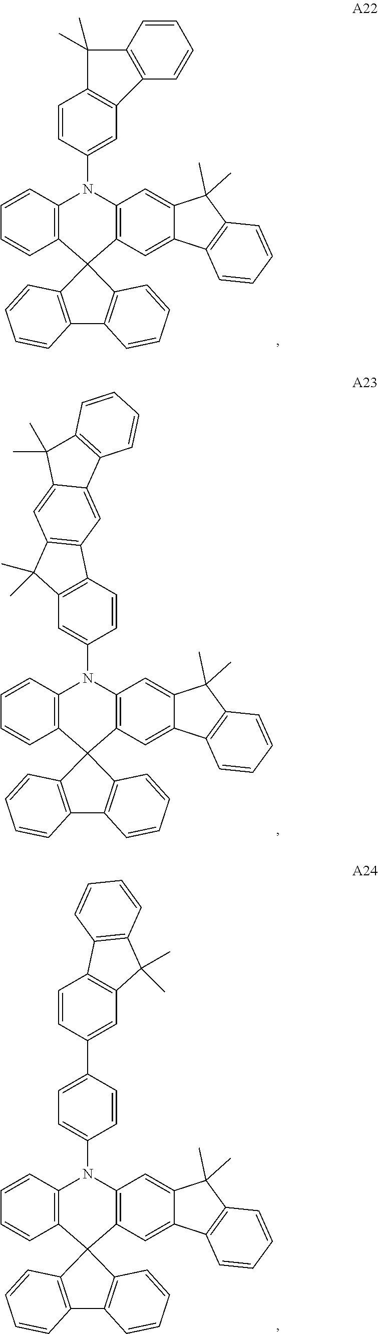 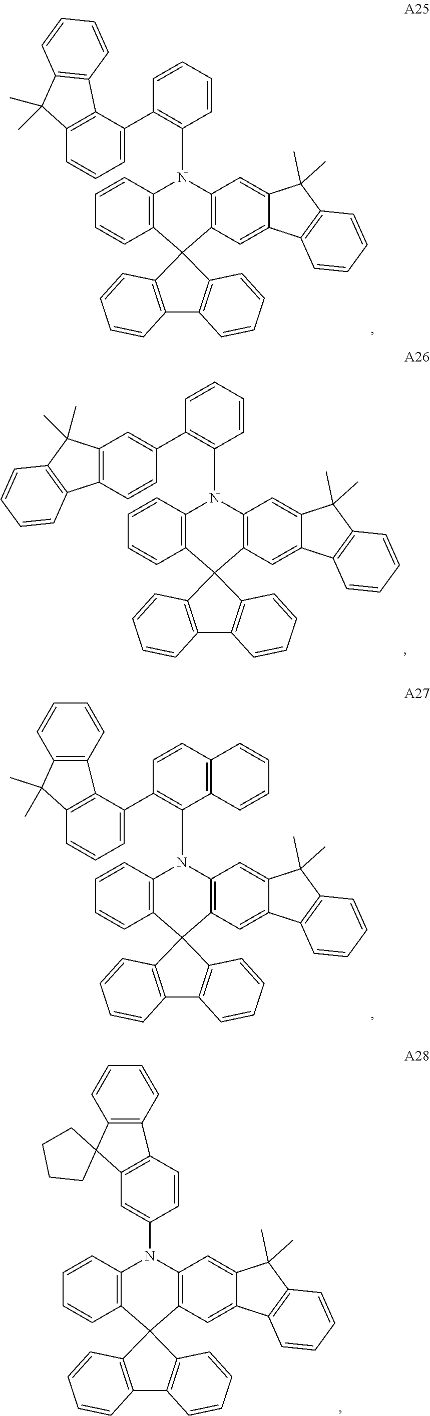 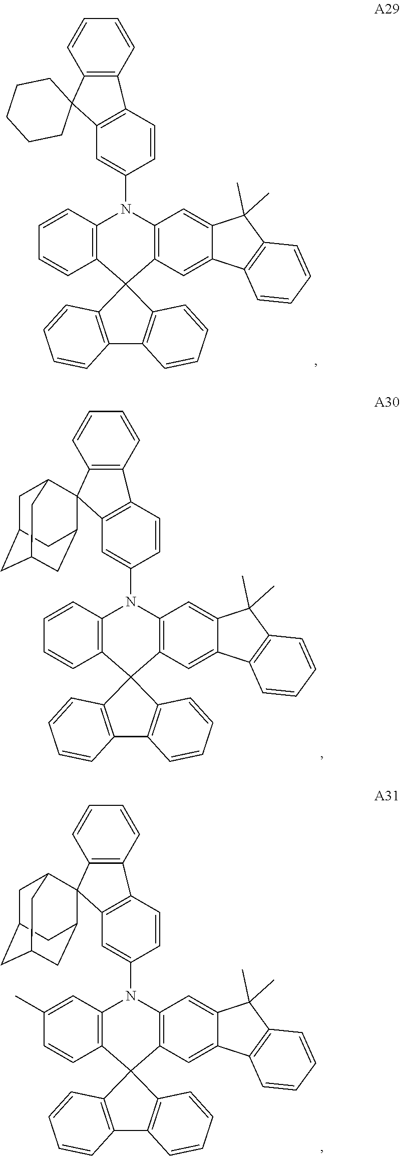 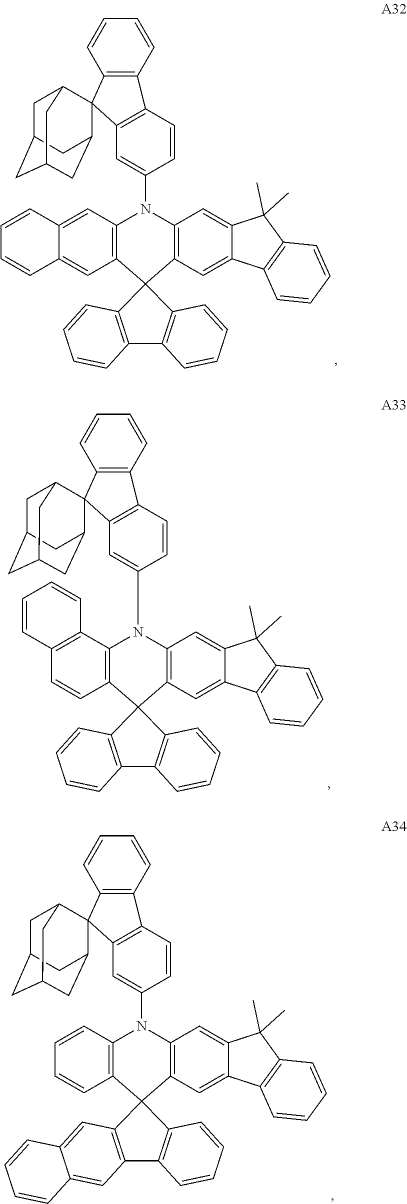 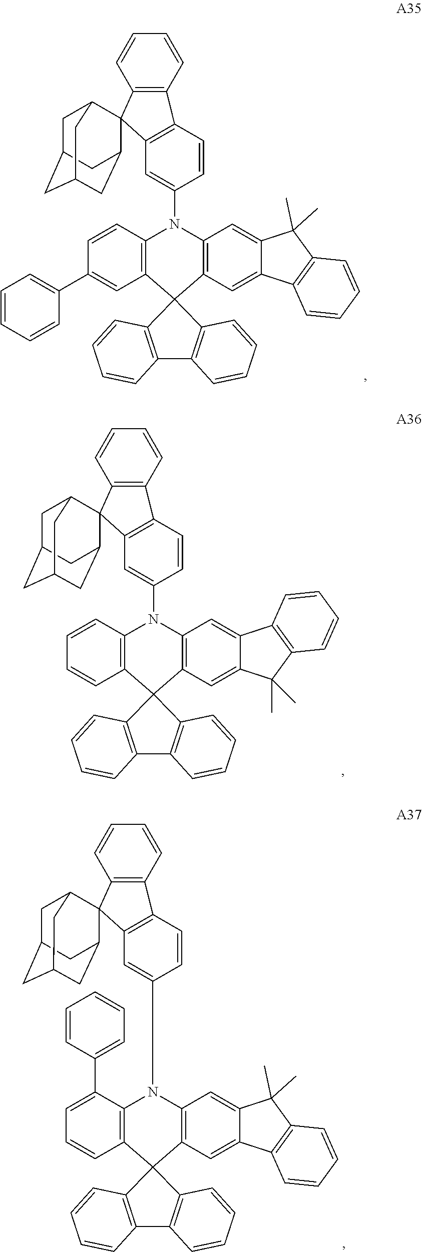 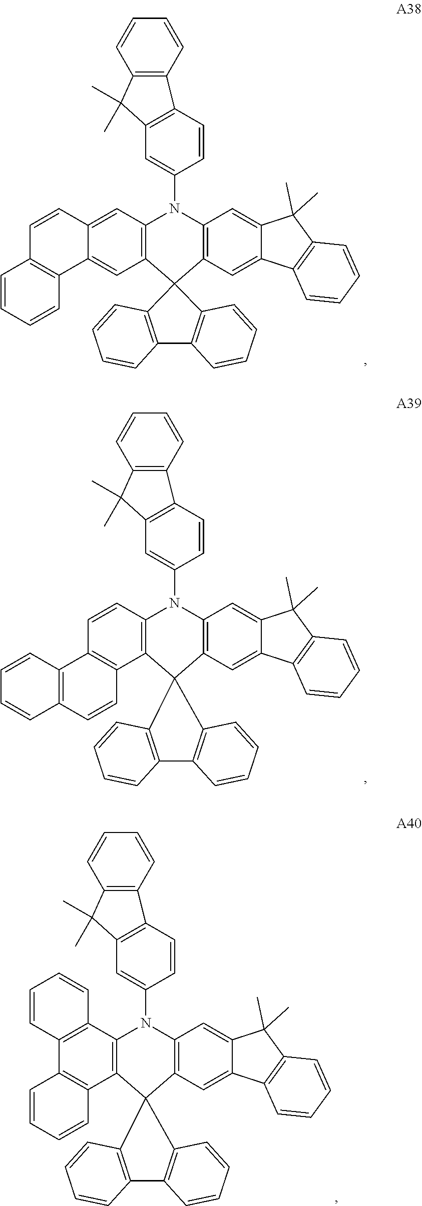 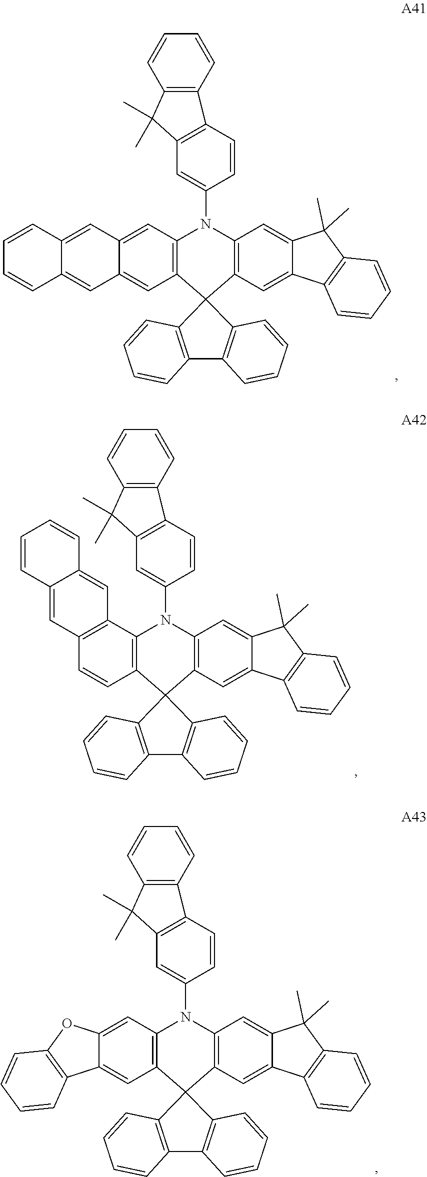 [SMILES: CC1(C)c2ccccc2-c2ccc(N3c4cc5c(cc4C4(c6ccccc6-c6ccccc64)c4cc6cc7ccccc7cc6cc43)-c3ccccc3C5(C)C)cc21 ; CC1(C)c2ccccc2-c2ccc(N3c4cc5c(cc4C4(c6ccccc6-c6ccccc64)c4ccc6cc7ccccc7cc6c43)-c3ccccc3C5(C)C)cc21 ; CC1(C)c2ccccc2-c2ccc(N3c4cc5c(cc4C4(c6ccccc6-c6ccccc64)c4cc6c(cc43)oc3ccccc36)-c3ccccc3C5(C)C)cc21] 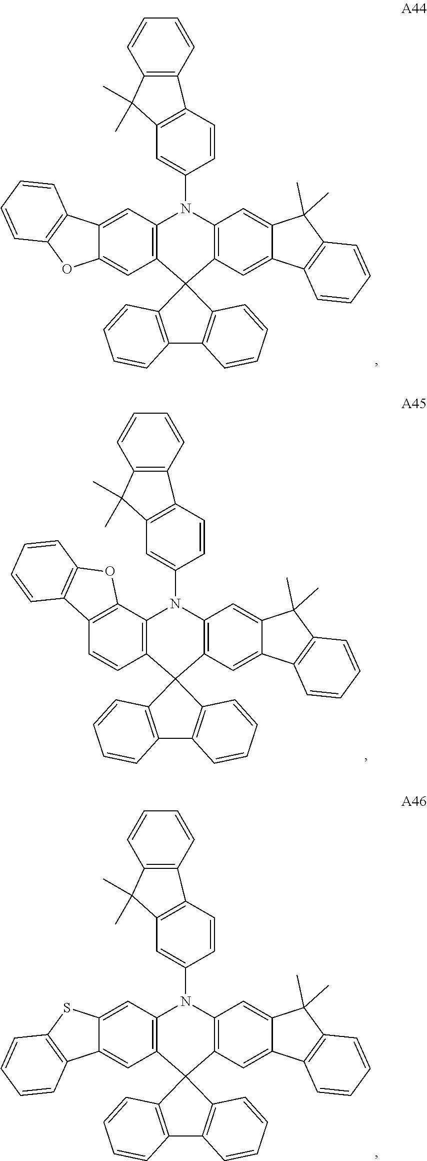 [SMILES: CC1(C)c2ccccc2-c2ccc(N3c4cc5c(cc4C4(c6ccccc6-c6ccccc64)c4cc6oc7ccccc7c6cc43)-c3ccccc3C5(C)C)cc21 ; CC1(C)c2ccccc2-c2ccc(N3c4cc5c(cc4C4(c6ccccc6-c6ccccc64)c4ccc6c(oc7ccccc76)c43)-c3ccccc3C5(C)C)cc21 ; CC1(C)c2ccccc2-c2ccc(N3c4cc5c(cc4C4(c6ccccc6-c6ccccc64)c4cc6c(cc43)sc3ccccc36)-c3ccccc3C5(C)C)cc21] 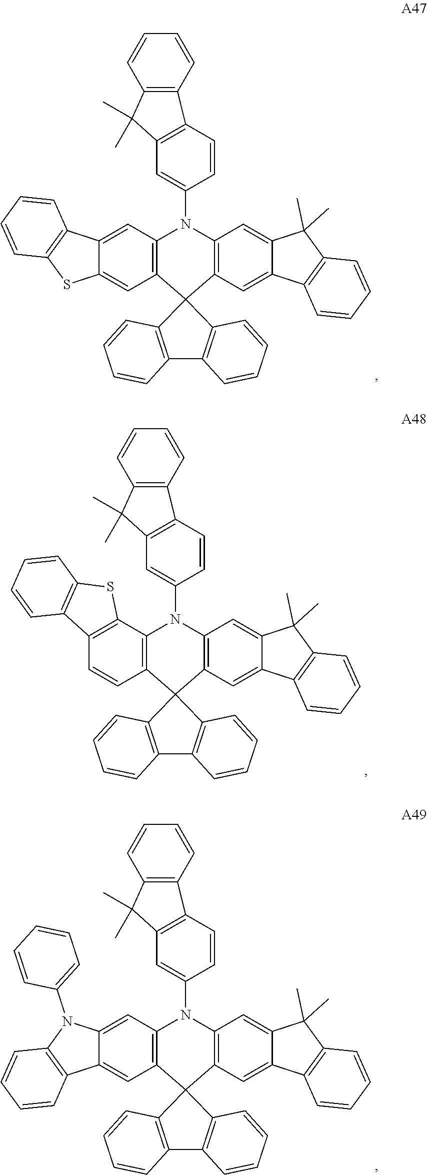 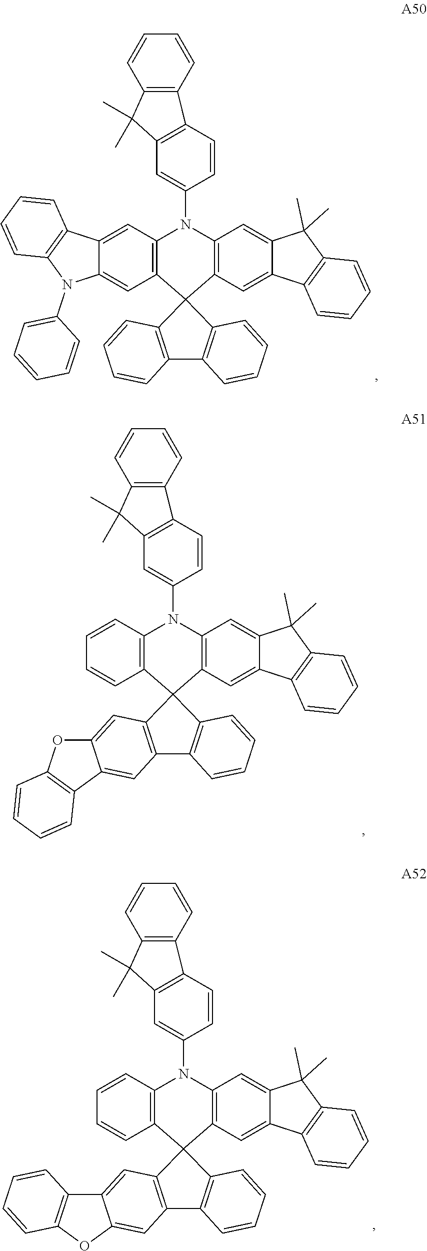 [SMILES: CC1(C)c2ccccc2-c2ccc(N3c4cc5c(cc4C4(c6ccccc6-c6ccccc64)c4cc6c(cc43)c3ccccc3n6-c3ccccc3)-c3ccccc3C5(C)C)cc21 ; CC1(C)c2ccccc2-c2ccc(N3c4ccccc4C4(c5ccccc5-c5cc6c(cc54)oc4ccccc46)c4cc5c(cc43)C(C)(C)c3ccccc3-5)cc21 ; CC1(C)c2ccccc2-c2ccc(N3c4ccccc4C4(c5ccccc5-c5cc6oc7ccccc7c6cc54)c4cc5c(cc43)C(C)(C)c3ccccc3-5)cc21] 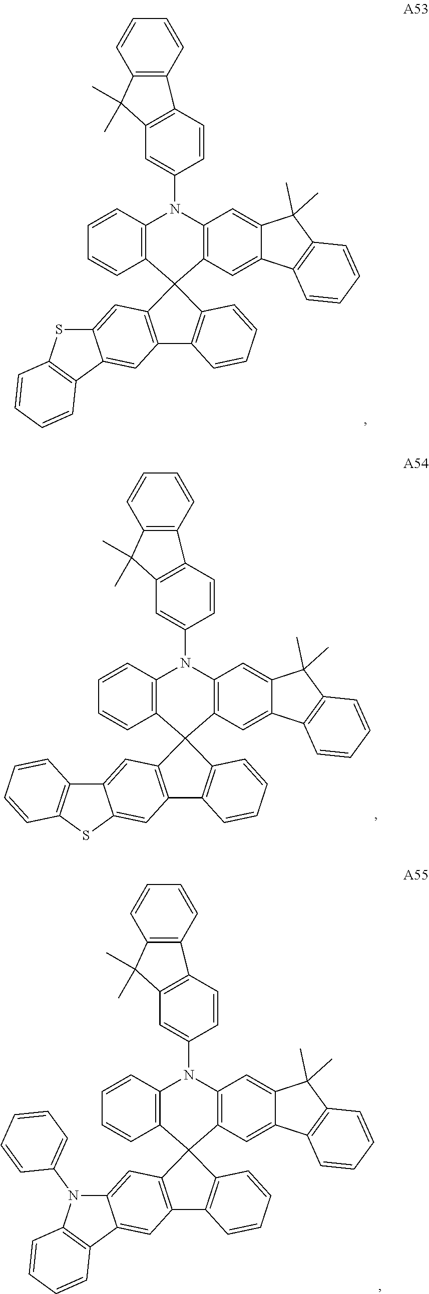 [SMILES: CC1(C)c2ccccc2-c2ccc(N3c4ccccc4C4(c5ccccc5-c5cc6c(cc54)sc4ccccc46)c4cc5c(cc43)C(C)(C)c3ccccc3-5)cc21 ; CC1(C)c2ccccc2-c2ccc(N3c4ccccc4C4(c5ccccc5-c5cc6sc7ccccc7c6cc54)c4cc5c(cc43)C(C)(C)c3ccccc3-5)cc21 ; CC1(C)c2ccccc2-c2ccc(N3c4ccccc4C4(c5ccccc5-c5cc6c7ccccc7n(-c7ccccc7)c6cc54)c4cc5c(cc43)C(C)(C)c3ccccc3-5)cc21] 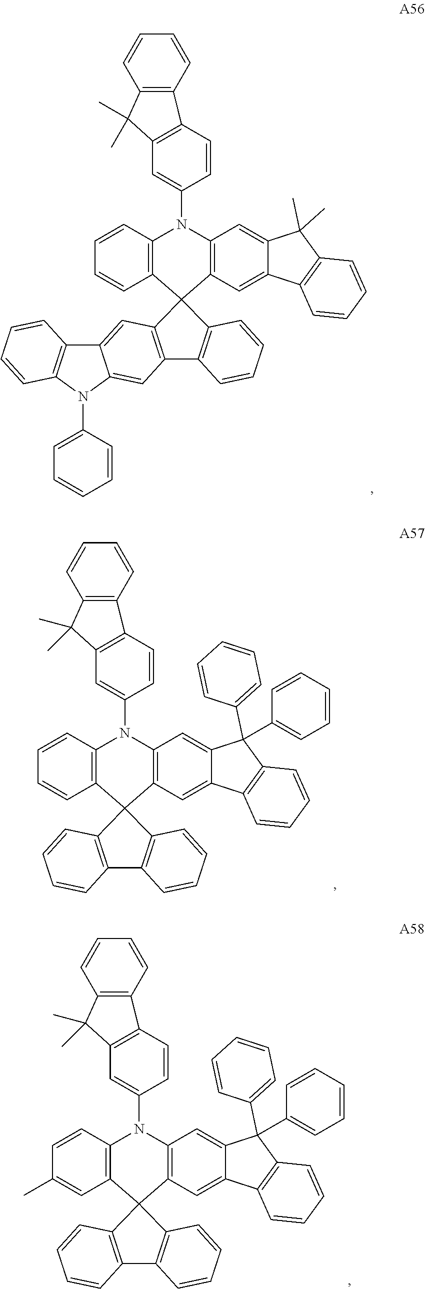 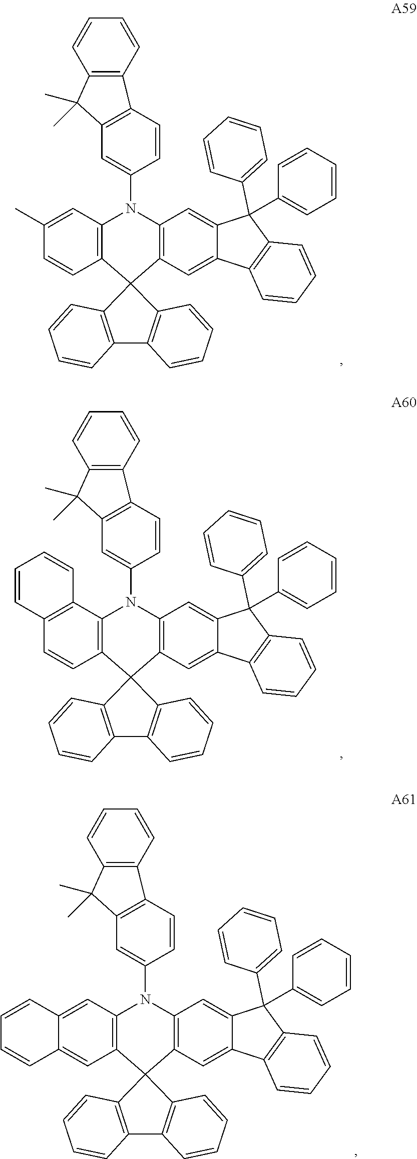 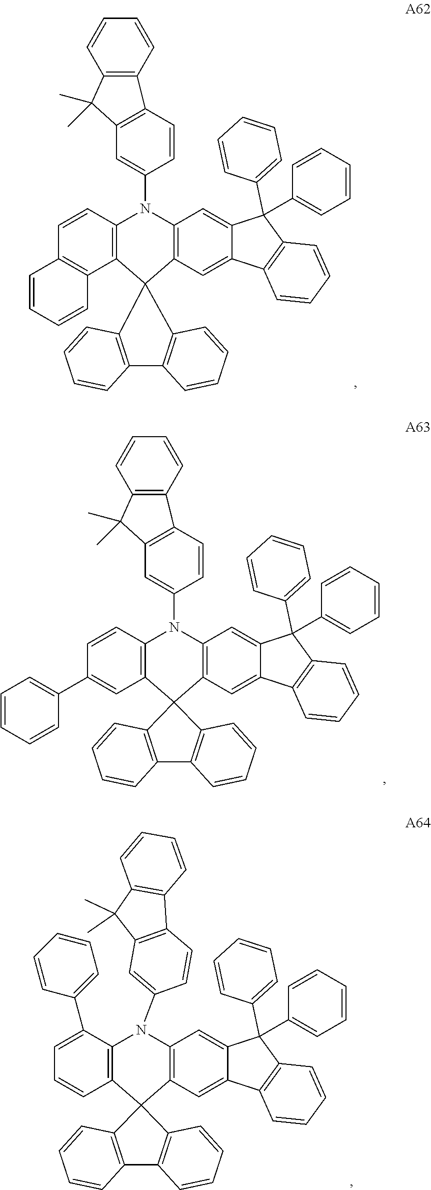 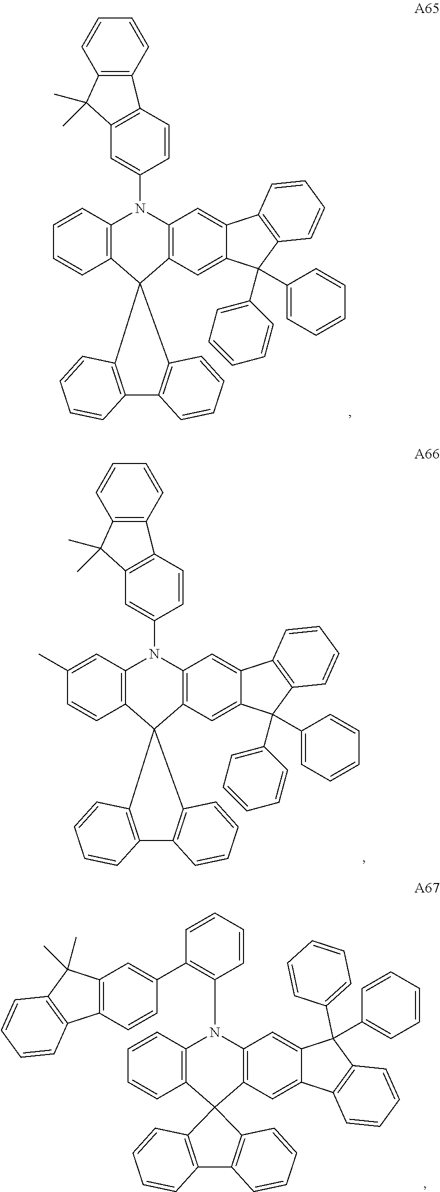 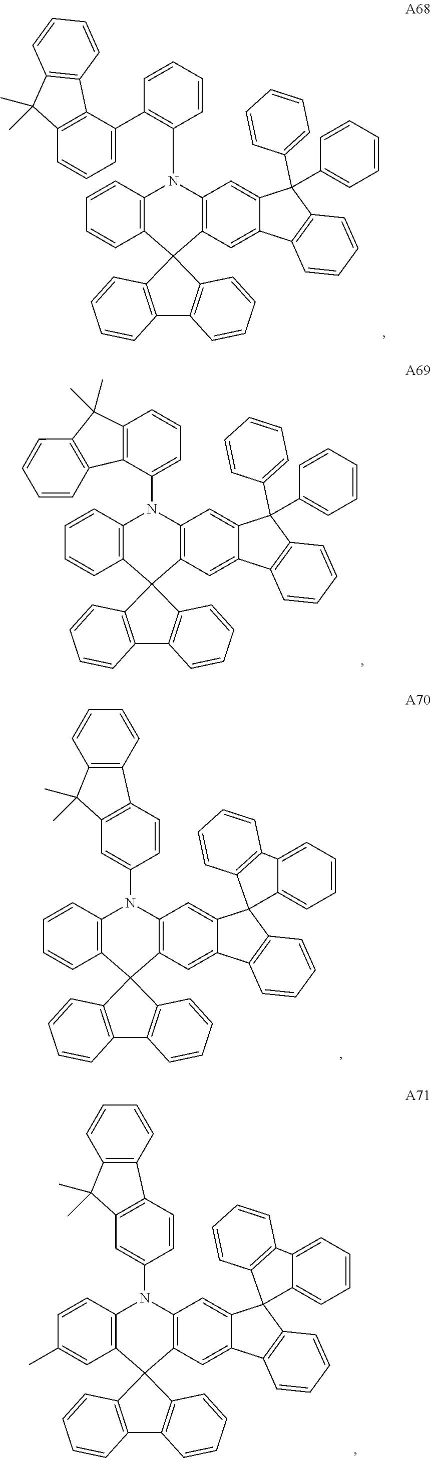 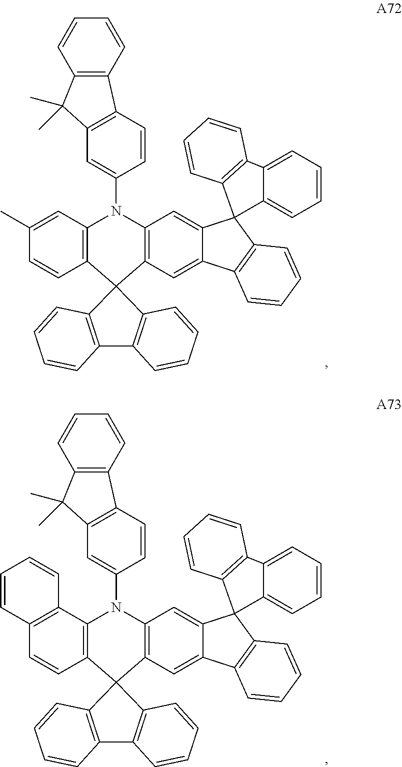 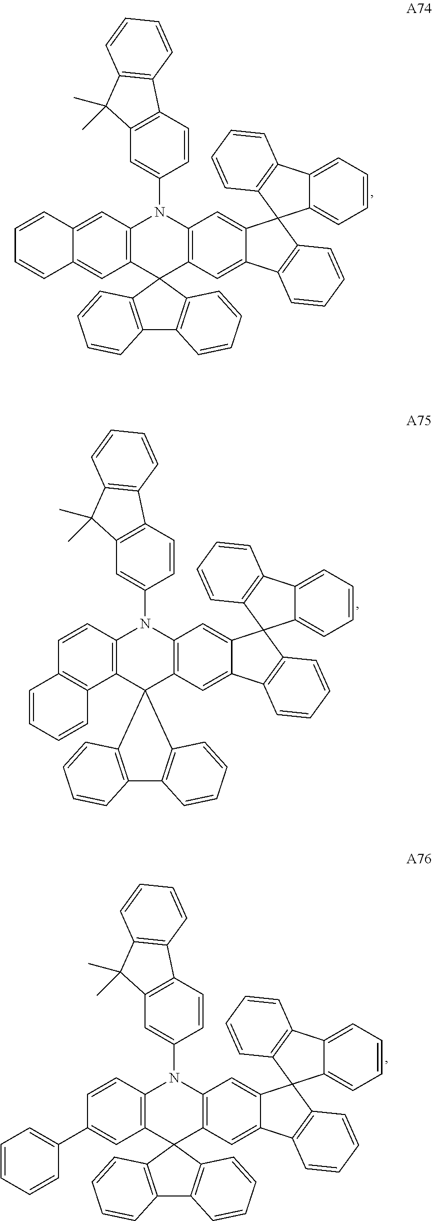 [SMILES: CC1(C)c2ccccc2-c2ccc(N3c4cc5c(cc4C4(c6ccccc6-c6ccccc64)c4cc6ccccc6cc43)-c3ccccc3C53c4ccccc4-c4ccccc43)cc21 ; CC1(C)c2ccccc2-c2ccc(N3c4cc5c(cc4C4(c6ccccc6-c6ccccc64)c4c3ccc3ccccc43)-c3ccccc3C53c4ccccc4-c4ccccc43)cc21 ; CC1(C)c2ccccc2-c2ccc(N3c4ccc(-c5ccccc5)cc4C4(c5ccccc5-c5ccccc54)c4cc5c(cc43)C3(c4ccccc4-c4ccccc43)c3ccccc3-5)cc21] 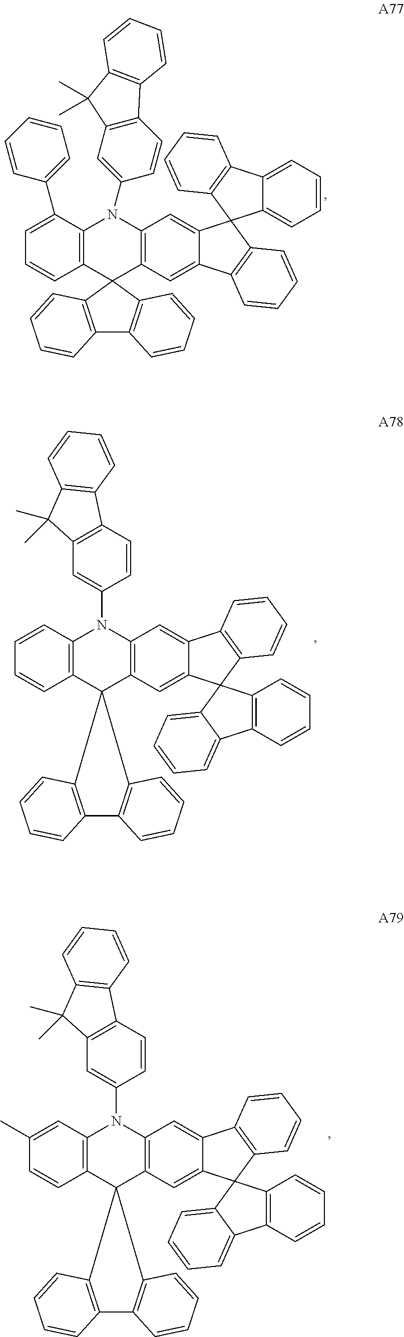 [SMILES: CC1(C)c2ccccc2-c2ccc(N3c4cc5c(cc4C4(c6ccccc6-c6ccccc64)c4cccc(-c6ccccc6)c43)-c3ccccc3C53c4ccccc4-c4ccccc43)cc21 ; CC1(C)c2ccccc2-c2ccc(N3c4ccccc4C4(c5ccccc5-c5ccccc54)c4cc5c(cc43)-c3ccccc3C53c4ccccc4-c4ccccc43)cc21 ; Cc1ccc2c(c1)N(c1ccc3c(c1)C(C)(C)c1ccccc1-3)c1cc3c(cc1C21c2ccccc2-c2ccccc21)C1(c2ccccc2-c2ccccc21)c1ccccc1-3] 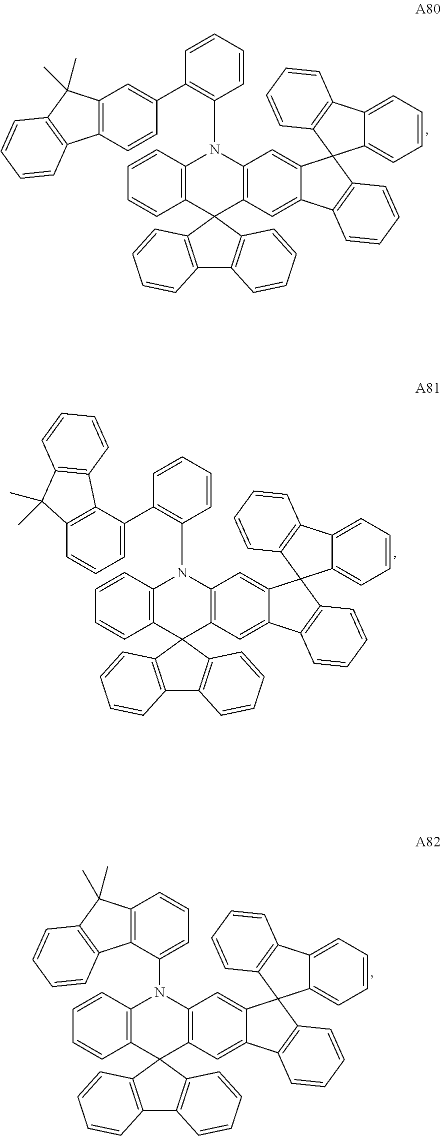 [SMILES: CC1(C)c2ccccc2-c2ccc(-c3ccccc3N3c4ccccc4C4(c5ccccc5-c5ccccc54)c4cc5c(cc43)C3(c4ccccc4-c4ccccc43)c3ccccc3-5)cc21 ; CC1(C)c2ccccc2-c2c(-c3ccccc3N3c4ccccc4C4(c5ccccc5-c5ccccc54)c4cc5c(cc43)C3(c4ccccc4-c4ccccc43)c3ccccc3-5)cccc21 ; CC1(C)c2ccccc2-c2c(N3c4ccccc4C4(c5ccccc5-c5ccccc54)c4cc5c(cc43)C3(c4ccccc4-c4ccccc43)c3ccccc3-5)cccc21] 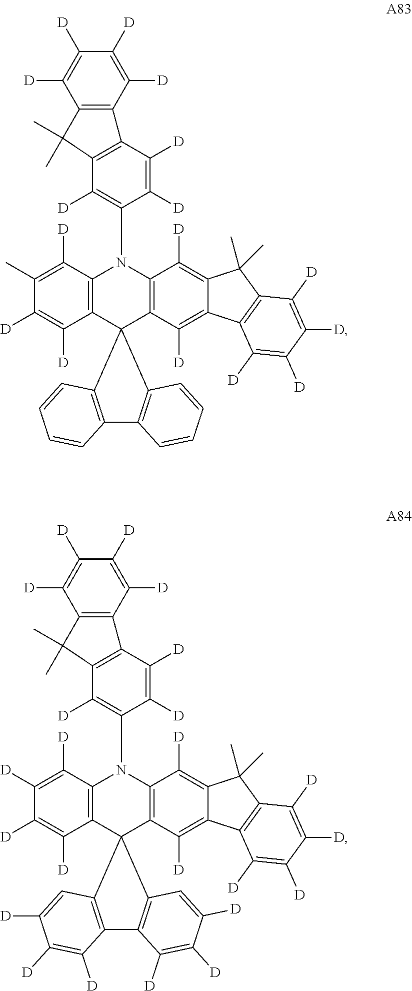 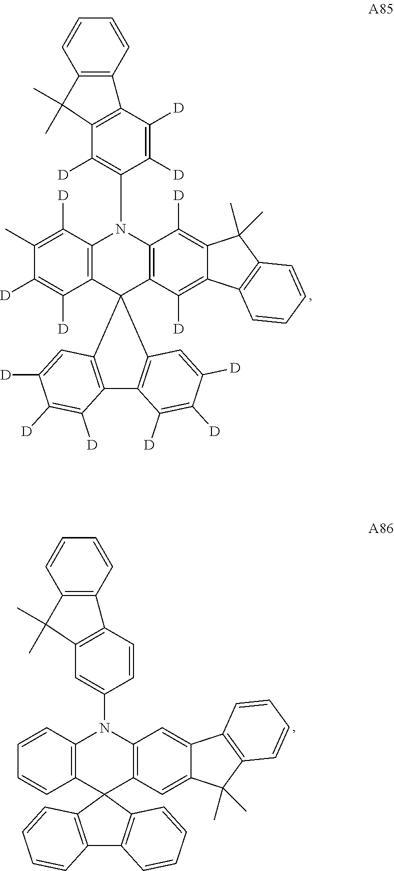 [SMILES: [2H]c1cc2c(c([2H])c1[2H])-c1c(cc([2H])c([2H])c1[2H])C21c2c([2H])c([2H])c(C)c([2H])c2N(c2c([2H])c([2H])c3c(c2[2H])C(C)(C)c2ccccc2-3)c2c([2H])c3c(c([2H])c21)-c1ccccc1C3(C)C ; CC1(C)c2ccccc2-c2ccc(N3c4ccccc4C4(c5ccccc5-c5ccccc54)c4cc5c(cc43)-c3ccccc3C5(C)C)cc21] 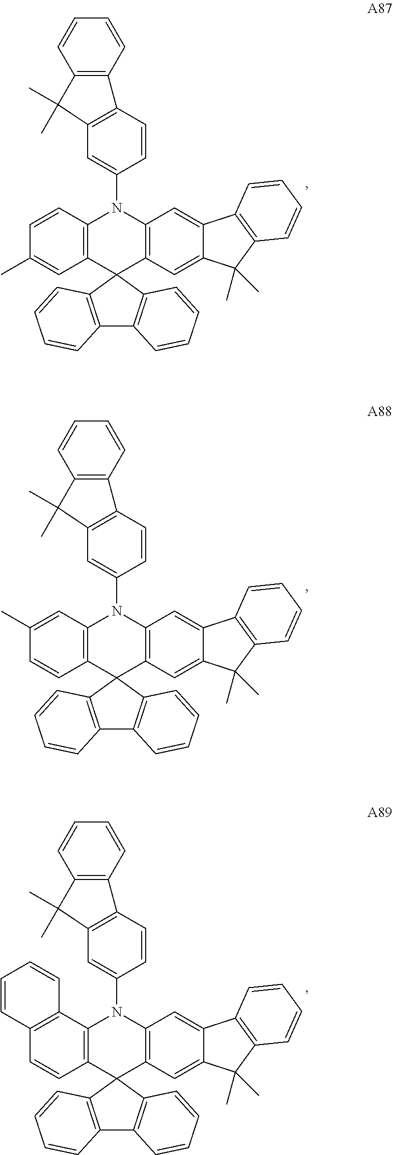 [SMILES: Cc1ccc2c(c1)C1(c3ccccc3-c3ccccc31)c1cc3c(cc1N2c1ccc2c(c1)C(C)(C)c1ccccc1-2)-c1ccccc1C3(C)C ; Cc1ccc2c(c1)N(c1ccc3c(c1)C(C)(C)c1ccccc1-3)c1cc3c(cc1C21c2ccccc2-c2ccccc21)C(C)(C)c1ccccc1-3 ; CC1(C)c2ccccc2-c2ccc(N3c4cc5c(cc4C4(c6ccccc6-c6ccccc64)c4ccc6ccccc6c43)C(C)(C)c3ccccc3-5)cc21] 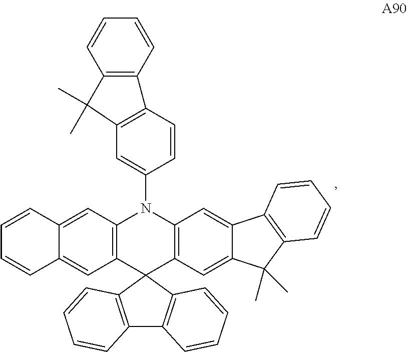 [SMILES: CC1(C)c2ccccc2-c2ccc(N3c4cc5c(cc4C4(c6ccccc6-c6ccccc64)c4cc6ccccc6cc43)C(C)(C)c3ccccc3-5)cc21] 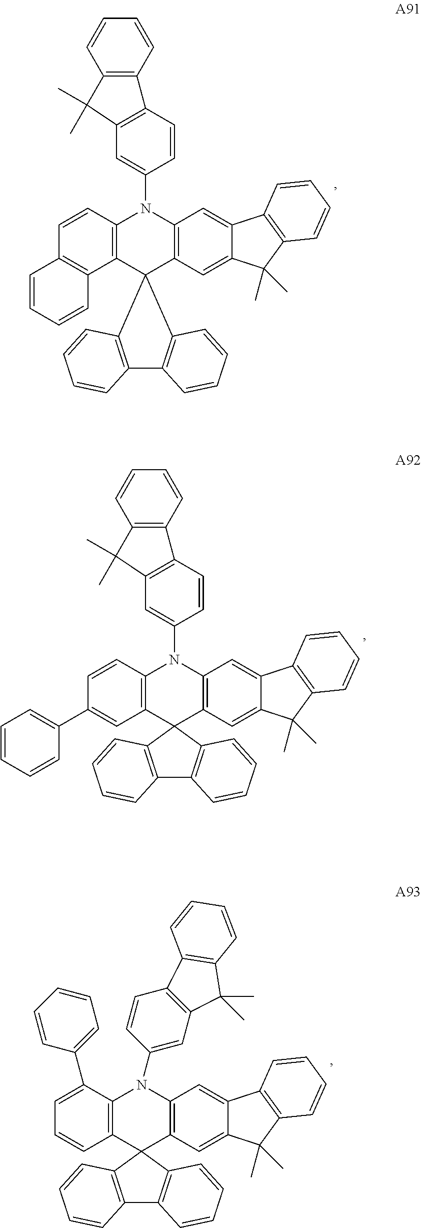 [SMILES: CC1(C)c2ccccc2-c2ccc(N3c4cc5c(cc4C4(c6ccccc6-c6ccccc64)c4c3ccc3ccccc43)C(C)(C)c3ccccc3-5)cc21 ; CC1(C)c2ccccc2-c2ccc(N3c4ccc(-c5ccccc5)cc4C4(c5ccccc5-c5ccccc54)c4cc5c(cc43)-c3ccccc3C5(C)C)cc21 ; CC1(C)c2ccccc2-c2ccc(N3c4cc5c(cc4C4(c6ccccc6-c6ccccc64)c4cccc(-c6ccccc6)c43)C(C)(C)c3ccccc3-5)cc21] 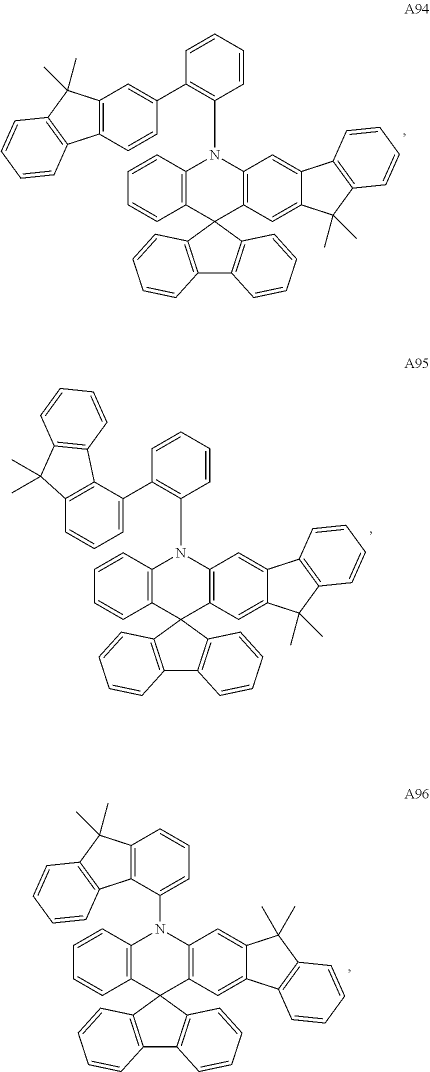 [SMILES: CC1(C)c2ccccc2-c2ccc(-c3ccccc3N3c4ccccc4C4(c5ccccc5-c5ccccc54)c4cc5c(cc43)-c3ccccc3C5(C)C)cc21 ; CC1(C)c2ccccc2-c2cc3c(cc21)C1(c2ccccc2-c2ccccc21)c1ccccc1N3c1ccccc1-c1cccc2c1-c1ccccc1C2(C)C ; CC1(C)c2ccccc2-c2cc3c(cc21)N(c1cccc2c1-c1ccccc1C2(C)C)c1ccccc1C31c2ccccc2-c2ccccc21] 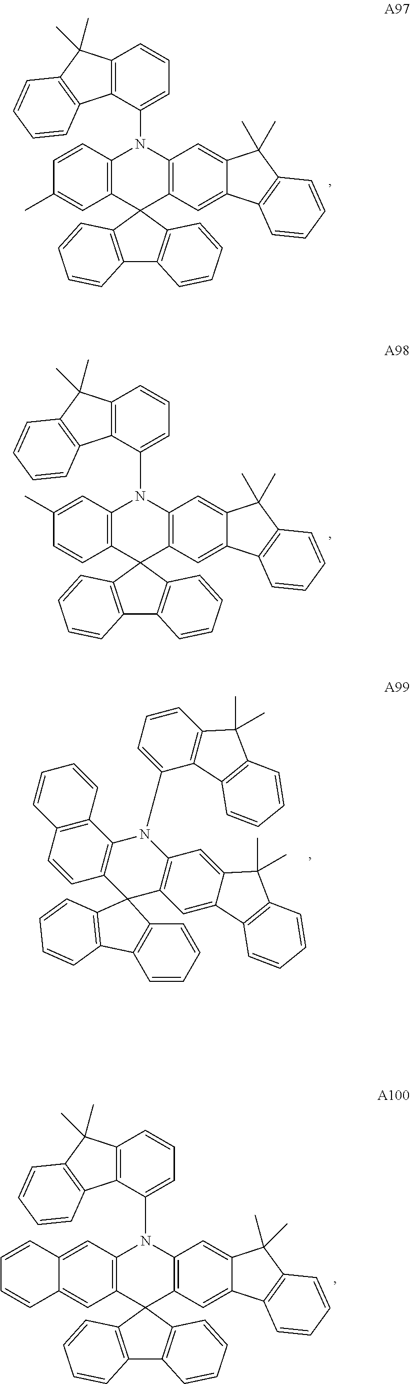 [SMILES: Cc1ccc2c(c1)C1(c3ccccc3-c3ccccc31)c1cc3c(cc1N2c1cccc2c1-c1ccccc1C2(C)C)C(C)(C)c1ccccc1-3 ; Cc1ccc2c(c1)N(c1cccc3c1-c1ccccc1C3(C)C)c1cc3c(cc1C21c2ccccc2-c2ccccc21)-c1ccccc1C3(C)C ; CC1(C)c2ccccc2-c2cc3c(cc21)N(c1cccc2c1-c1ccccc1C2(C)C)c1c(ccc2ccccc12)C31c2ccccc2-c2ccccc21 ; CC1(C)c2ccccc2-c2cc3c(cc21)N(c1cccc2c1-c1ccccc1C2(C)C)c1cc2ccccc2cc1C31c2ccccc2-c2ccccc21] 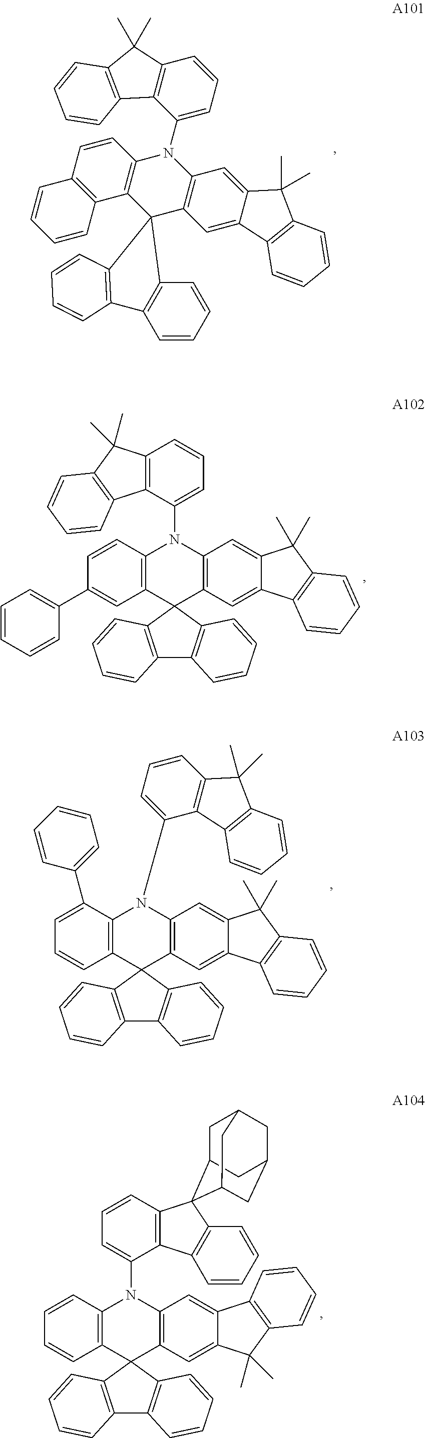 [SMILES: CC1(C)c2ccccc2-c2cc3c(cc21)N(c1cccc2c1-c1ccccc1C2(C)C)c1ccc2ccccc2c1C31c2ccccc2-c2ccccc21 ; CC1(C)c2ccccc2-c2cc3c(cc21)N(c1cccc2c1-c1ccccc1C2(C)C)c1ccc(-c2ccccc2)cc1C31c2ccccc2-c2ccccc21 ; CC1(C)c2ccccc2-c2cc3c(cc21)N(c1cccc2c1-c1ccccc1C2(C)C)c1c(-c2ccccc2)cccc1C31c2ccccc2-c2ccccc21 ; CC1(C)c2ccccc2-c2cc3c(cc21)C1(c2ccccc2-c2ccccc21)c1ccccc1N3c1cccc2c1-c1ccccc1C21C2CC3CC(C2)CC1C3] 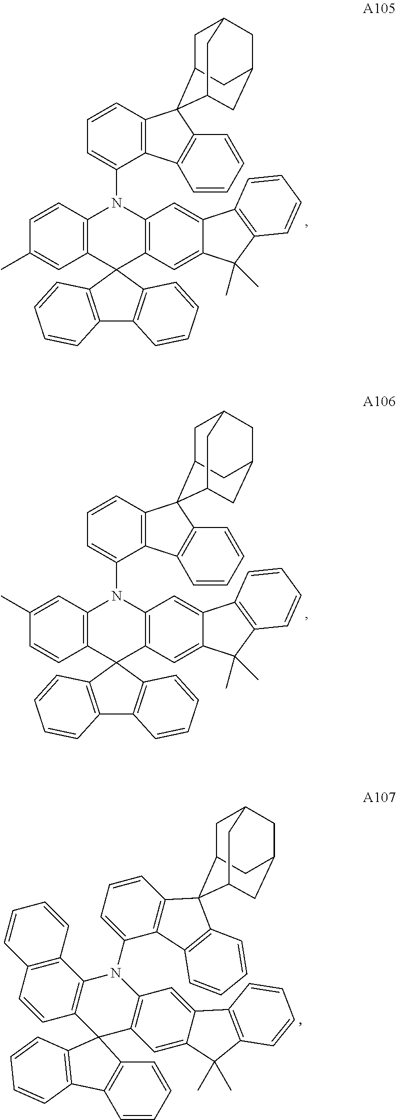 [SMILES: Cc1ccc2c(c1)C1(c3ccccc3-c3ccccc31)c1cc3c(cc1N2c1cccc2c1-c1ccccc1C21C2CC4CC(C2)CC1C4)-c1ccccc1C3(C)C ; Cc1ccc2c(c1)N(c1cccc3c1-c1ccccc1C31C3CC4CC(C3)CC1C4)c1cc3c(cc1C21c2ccccc2-c2ccccc21)C(C)(C)c1ccccc1-3 ; CC1(C)c2ccccc2-c2cc3c(cc21)C1(c2ccccc2-c2ccccc21)c1ccc2ccccc2c1N3c1cccc2c1-c1ccccc1C21C2CC3CC(C2)CC1C3] 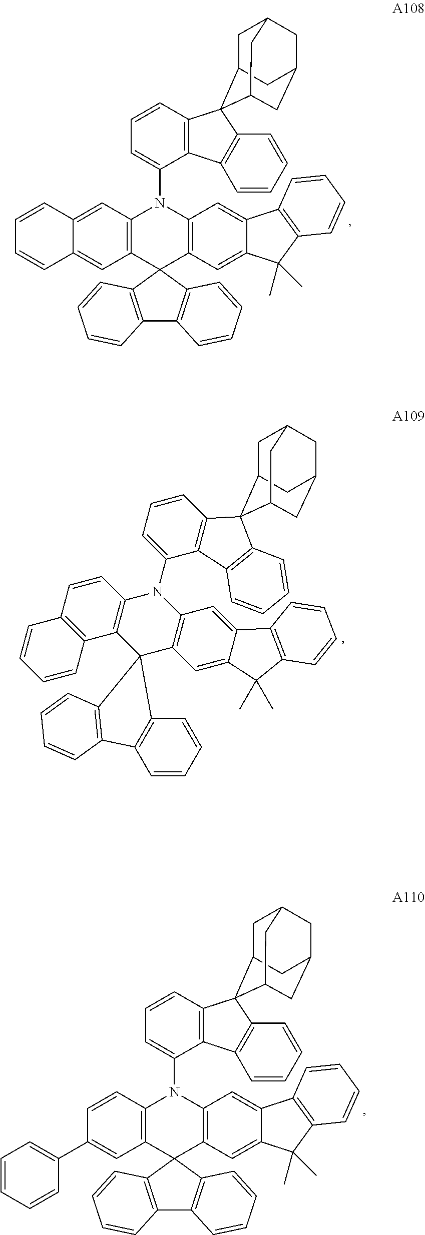 [SMILES: CC1(C)c2ccccc2-c2cc3c(cc21)C1(c2ccccc2-c2ccccc21)c1cc2ccccc2cc1N3c1cccc2c1-c1ccccc1C21C2CC3CC(C2)CC1C3 ; CC1(C)c2ccccc2-c2cc3c(cc21)C1(c2ccccc2-c2ccccc21)c1c(ccc2ccccc12)N3c1cccc2c1-c1ccccc1C21C2CC3CC(C2)CC1C3 ; CC1(C)c2ccccc2-c2cc3c(cc21)C1(c2ccccc2-c2ccccc21)c1cc(-c2ccccc2)ccc1N3c1cccc2c1-c1ccccc1C21C2CC3CC(C2)CC1C3] 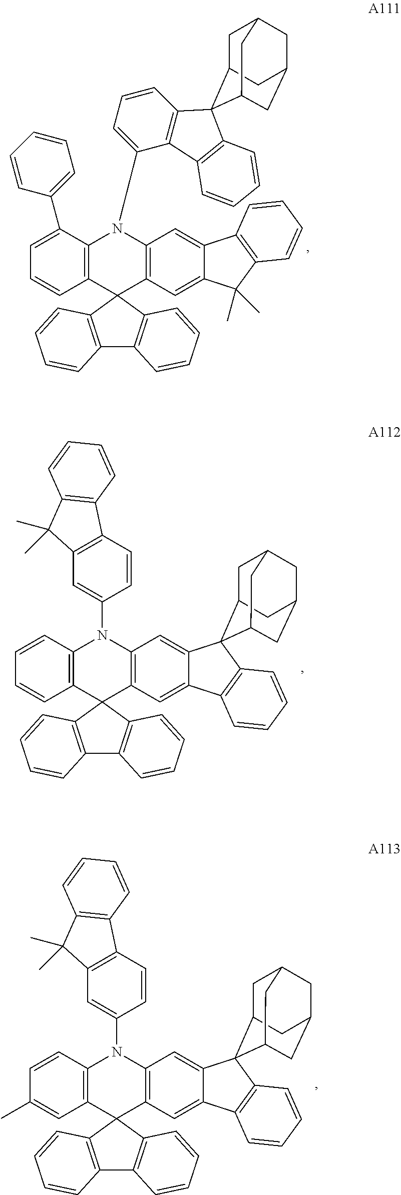 [SMILES: CC1(C)c2ccccc2-c2cc3c(cc21)C1(c2ccccc2-c2ccccc21)c1cccc(-c2ccccc2)c1N3c1cccc2c1-c1ccccc1C21C2CC3CC(C2)CC1C3 ; CC1(C)c2ccccc2-c2ccc(N3c4ccccc4C4(c5ccccc5-c5ccccc54)c4cc5c(cc43)C3(c4ccccc4-5)C4CC5CC(C4)CC3C5)cc21 ; Cc1ccc2c(c1)C1(c3ccccc3-c3ccccc31)c1cc3c(cc1N2c1ccc2c(c1)C(C)(C)c1ccccc1-2)C1(c2ccccc2-3)C2CC3CC(C2)CC1C3] 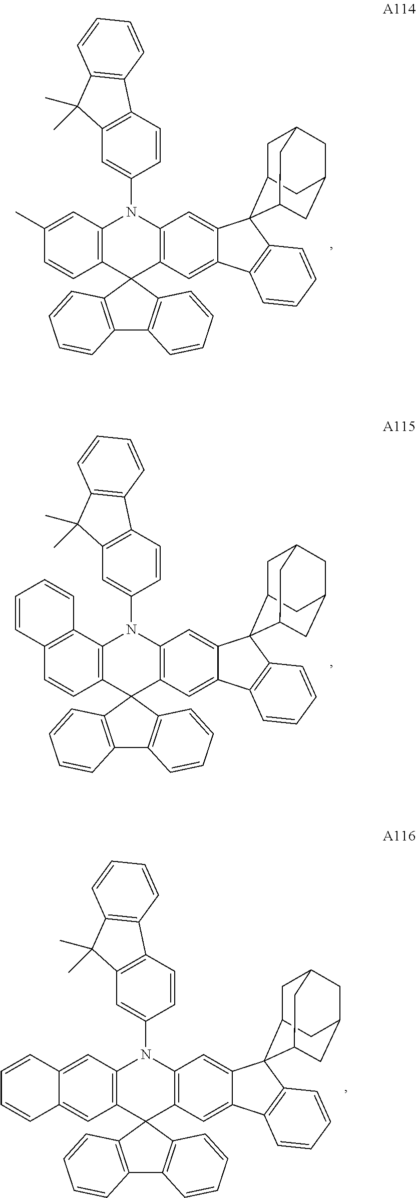 [SMILES: Cc1ccc2c(c1)N(c1ccc3c(c1)C(C)(C)c1ccccc1-3)c1cc3c(cc1C21c2ccccc2-c2ccccc21)-c1ccccc1C31C2CC3CC(C2)CC1C3 ; CC1(C)c2ccccc2-c2ccc(N3c4cc5c(cc4C4(c6ccccc6-c6ccccc64)c4ccc6ccccc6c43)-c3ccccc3C53C4CC5CC(C4)CC3C5)cc21 ; CC1(C)c2ccccc2-c2ccc(N3c4cc5c(cc4C4(c6ccccc6-c6ccccc64)c4cc6ccccc6cc43)-c3ccccc3C53C4CC5CC(C4)CC3C5)cc21] 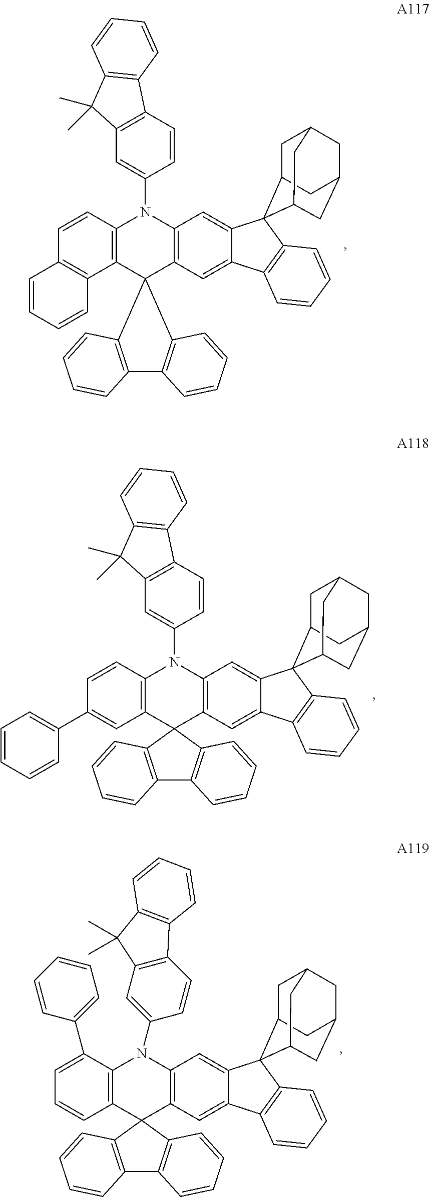 [SMILES: CC1(C)c2ccccc2-c2ccc(N3c4cc5c(cc4C4(c6ccccc6-c6ccccc64)c4c3ccc3ccccc43)-c3ccccc3C53C4CC5CC(C4)CC3C5)cc21 ; CC1(C)c2ccccc2-c2ccc(N3c4ccc(-c5ccccc5)cc4C4(c5ccccc5-c5ccccc54)c4cc5c(cc43)C3(c4ccccc4-5)C4CC5CC(C4)CC3C5)cc21 ; CC1(C)c2ccccc2-c2ccc(N3c4cc5c(cc4C4(c6ccccc6-c6ccccc64)c4cccc(-c6ccccc6)c43)-c3ccccc3C53C4CC5CC(C4)CC3C5)cc21] 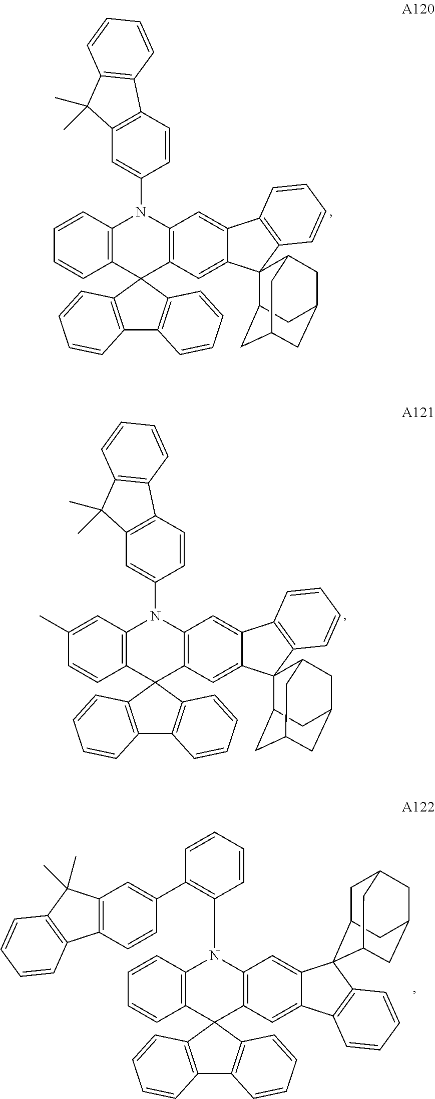 [SMILES: CC1(C)c2ccccc2-c2ccc(N3c4ccccc4C4(c5ccccc5-c5ccccc54)c4cc5c(cc43)-c3ccccc3C53C4CC5CC(C4)CC3C5)cc21 ; Cc1ccc2c(c1)N(c1ccc3c(c1)C(C)(C)c1ccccc1-3)c1cc3c(cc1C21c2ccccc2-c2ccccc21)C1(c2ccccc2-3)C2CC3CC(C2)CC1C3 ; CC1(C)c2ccccc2-c2ccc(-c3ccccc3N3c4ccccc4C4(c5ccccc5-c5ccccc54)c4cc5c(cc43)C3(c4ccccc4-5)C4CC5CC(C4)CC3C5)cc21] 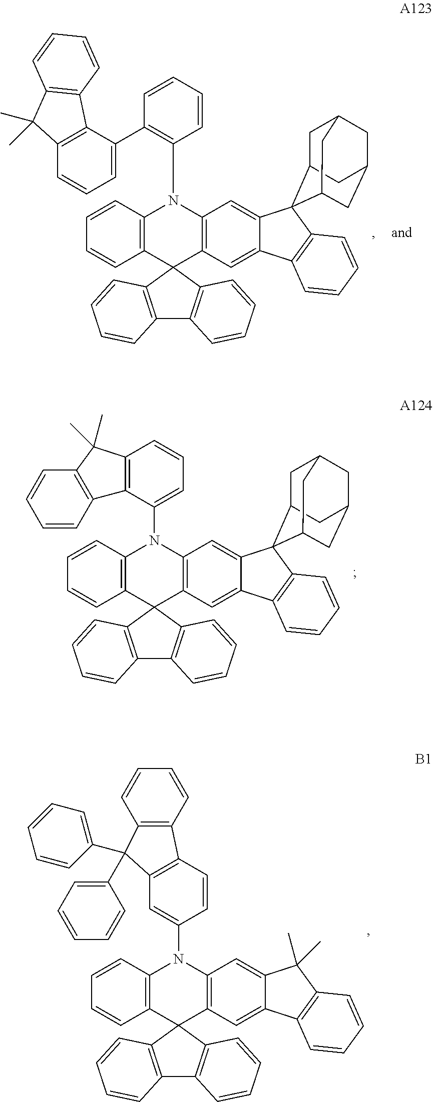 [SMILES: CC1(C)c2ccccc2-c2c(-c3ccccc3N3c4ccccc4C4(c5ccccc5-c5ccccc54)c4cc5c(cc43)C3(c4ccccc4-5)C4CC5CC(C4)CC3C5)cccc21 ; CC1(C)c2ccccc2-c2c(N3c4ccccc4C4(c5ccccc5-c5ccccc54)c4cc5c(cc43)C3(c4ccccc4-5)C4CC5CC(C4)CC3C5)cccc21 ; CC1(C)c2ccccc2-c2cc3c(cc21)N(c1ccc2c(c1)C(c1ccccc1)(c1ccccc1)c1ccccc1-2)c1ccccc1C31c2ccccc2-c2ccccc21] 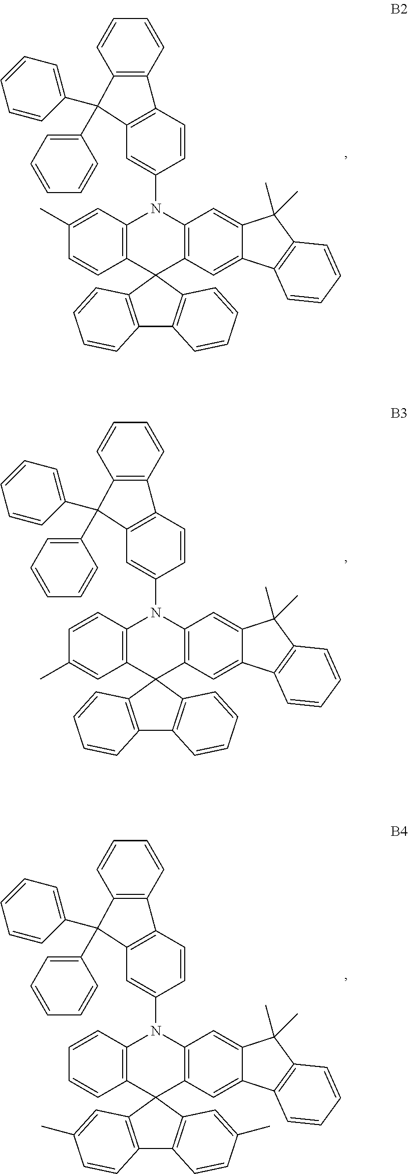 [SMILES: Cc1ccc2c(c1)N(c1ccc3c(c1)C(c1ccccc1)(c1ccccc1)c1ccccc1-3)c1cc3c(cc1C21c2ccccc2-c2ccccc21)-c1ccccc1C3(C)C ; Cc1ccc2c(c1)C1(c3ccccc3-c3ccccc31)c1cc3c(cc1N2c1ccc2c(c1)C(c1ccccc1)(c1ccccc1)c1ccccc1-2)C(C)(C)c1ccccc1-3 ; Cc1ccc2c(c1)C1(c3cc(C)ccc3-2)c2ccccc2N(c2ccc3c(c2)C(c2ccccc2)(c2ccccc2)c2ccccc2-3)c2cc3c(cc21)-c1ccccc1C3(C)C] 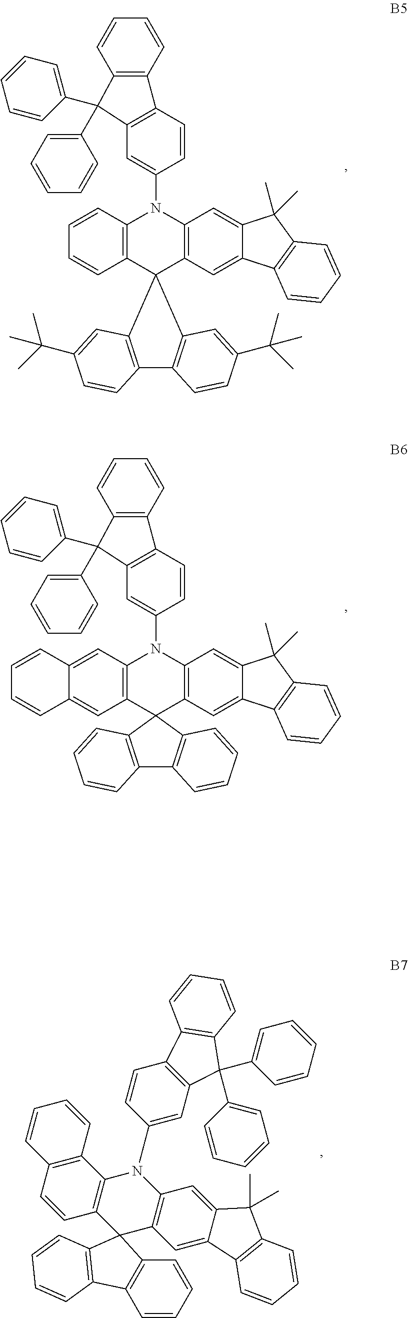 [SMILES: CC(C)(C)c1ccc2c(c1)C1(c3cc(C(C)(C)C)ccc3-2)c2ccccc2N(c2ccc3c(c2)C(c2ccccc2)(c2ccccc2)c2ccccc2-3)c2cc3c(cc21)-c1ccccc1C3(C)C ; CC1(C)c2ccccc2-c2cc3c(cc21)N(c1ccc2c(c1)C(c1ccccc1)(c1ccccc1)c1ccccc1-2)c1cc2ccccc2cc1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1c(ccc2ccccc12)C31c2ccccc2-c2ccccc21] 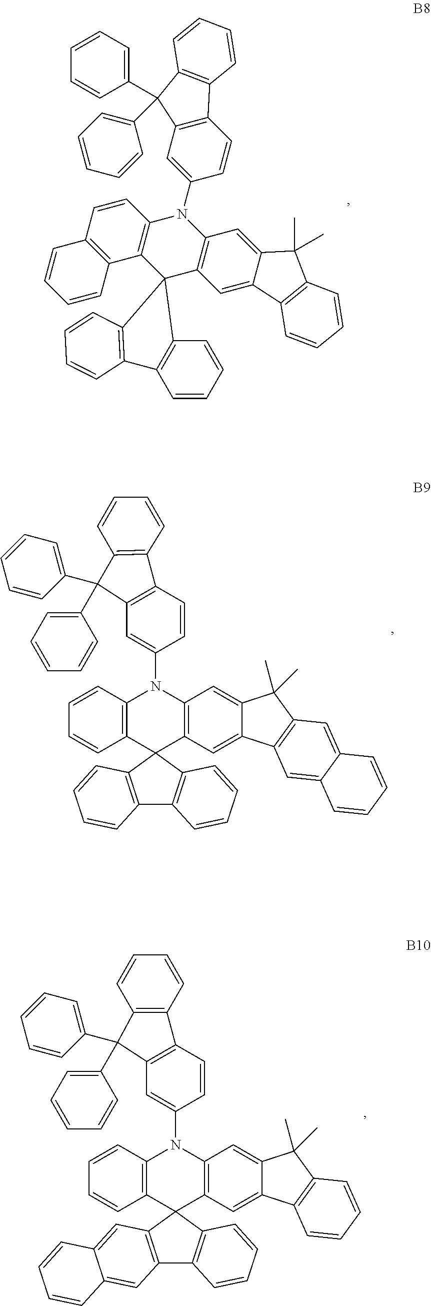 [SMILES: CC1(C)c2ccccc2-c2cc3c(cc21)N(c1ccc2c(c1)C(c1ccccc1)(c1ccccc1)c1ccccc1-2)c1ccc2ccccc2c1C31c2ccccc2-c2ccccc21 ; CC1(C)c2cc3c(cc2-c2cc4ccccc4cc21)C1(c2ccccc2-c2ccccc21)c1ccccc1N3c1ccc2c(c1)C(c1ccccc1)(c1ccccc1)c1ccccc1-2 ; CC1(C)c2ccccc2-c2cc3c(cc21)N(c1ccc2c(c1)C(c1ccccc1)(c1ccccc1)c1ccccc1-2)c1ccccc1C31c2ccccc2-c2cc3ccccc3cc21] 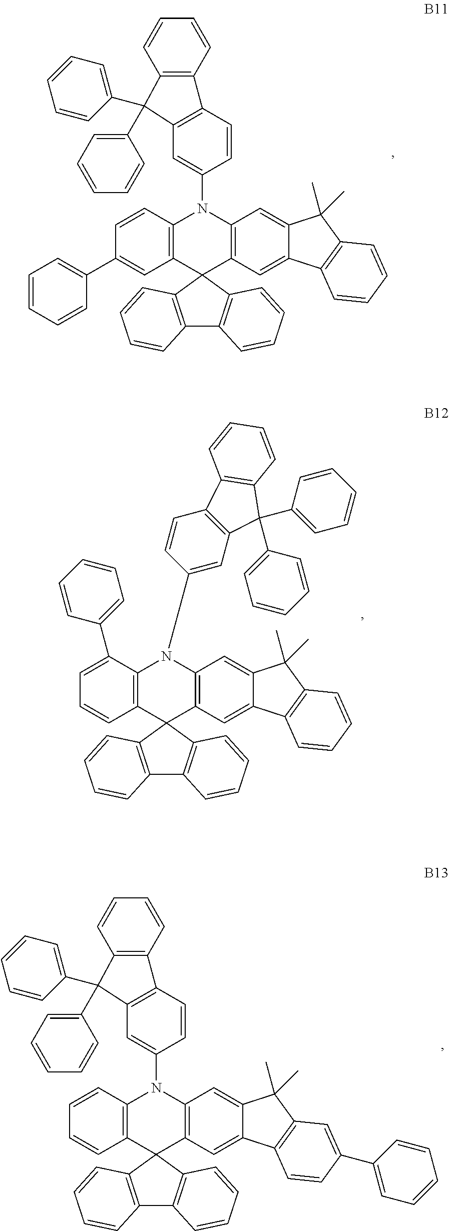 [SMILES: CC1(C)c2ccccc2-c2cc3c(cc21)N(c1ccc2c(c1)C(c1ccccc1)(c1ccccc1)c1ccccc1-2)c1ccc(-c2ccccc2)cc1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1c(-c2ccccc2)cccc1C31c2ccccc2-c2ccccc21 ; CC1(C)c2cc(-c3ccccc3)ccc2-c2cc3c(cc21)N(c1ccc2c(c1)C(c1ccccc1)(c1ccccc1)c1ccccc1-2)c1ccccc1C31c2ccccc2-c2ccccc21] 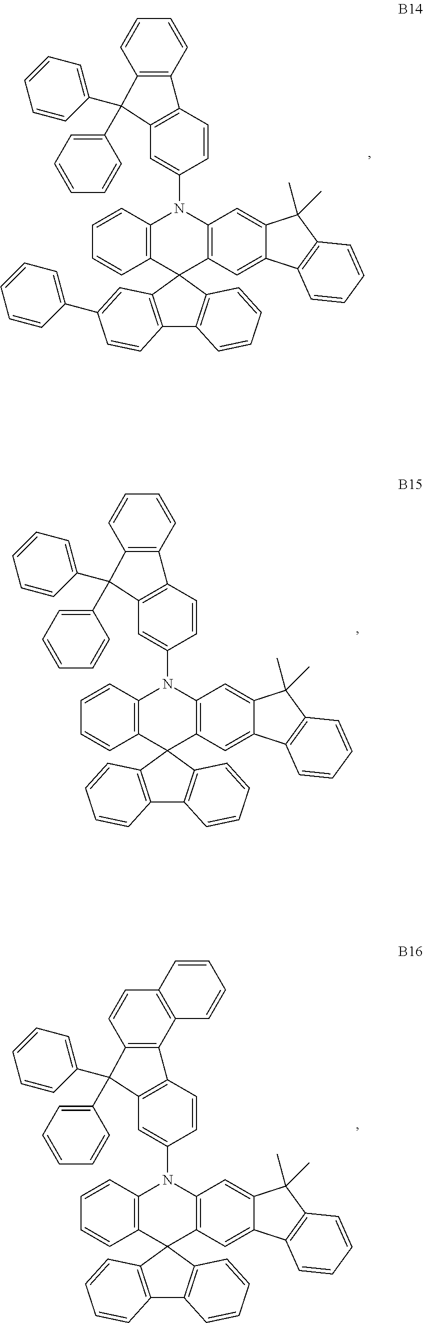 [SMILES: CC1(C)c2ccccc2-c2cc3c(cc21)N(c1ccc2c(c1)C(c1ccccc1)(c1ccccc1)c1ccccc1-2)c1ccccc1C31c2ccccc2-c2ccc(-c3ccccc3)cc21 ; CC1(C)c2ccccc2-c2cc3c(cc21)N(c1ccc2c(c1)C(c1ccccc1)(c1ccccc1)c1ccccc1-2)c1ccccc1C31c2ccccc2-c2ccccc21 ; CC1(C)c2ccccc2-c2cc3c(cc21)N(c1ccc2c(c1)C(c1ccccc1)(c1ccccc1)c1ccc4ccccc4c1-2)c1ccccc1C31c2ccccc2-c2ccccc21] 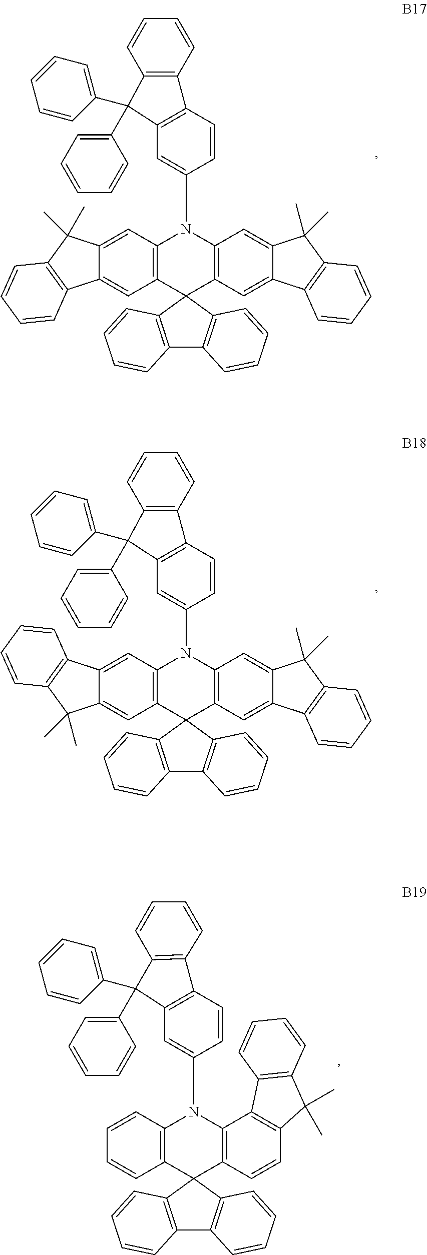 [SMILES: CC1(C)c2ccccc2-c2cc3c(cc21)N(c1ccc2c(c1)C(c1ccccc1)(c1ccccc1)c1ccccc1-2)c1cc2c(cc1C31c3ccccc3-c3ccccc31)-c1ccccc1C2(C)C ; CC1(C)c2ccccc2-c2cc3c(cc21)N(c1ccc2c(c1)C(c1ccccc1)(c1ccccc1)c1ccccc1-2)c1cc2c(cc1C31c3ccccc3-c3ccccc31)C(C)(C)c1ccccc1-2 ; CC1(C)c2ccccc2-c2c1ccc1c2N(c2ccc3c(c2)C(c2ccccc2)(c2ccccc2)c2ccccc2-3)c2ccccc2C12c1ccccc1-c1ccccc12] 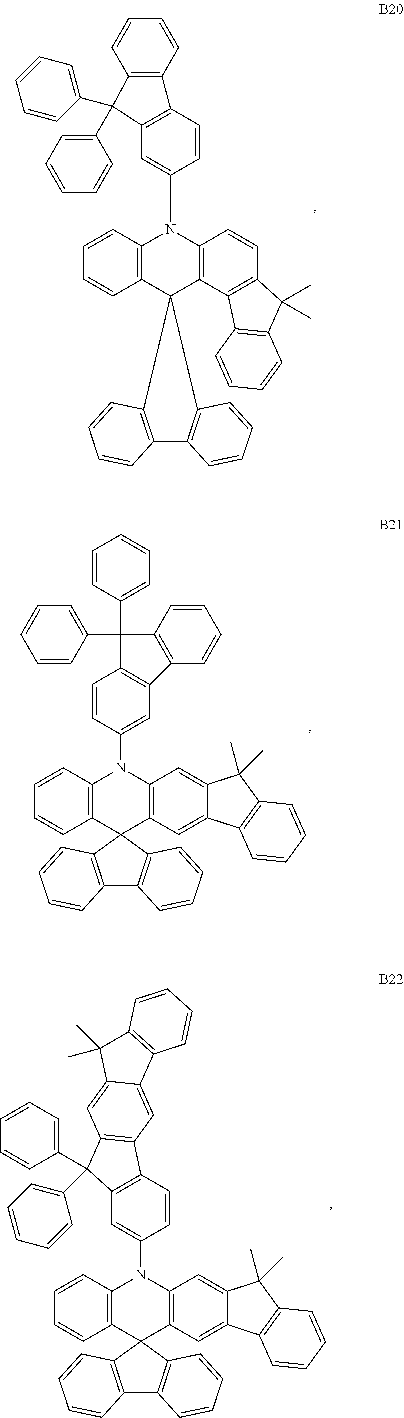 [SMILES: CC1(C)c2ccccc2-c2c1ccc1c2C2(c3ccccc3-c3ccccc32)c2ccccc2N1c1ccc2c(c1)C(c1ccccc1)(c1ccccc1)c1ccccc1-2 ; CC1(C)c2ccccc2-c2cc3c(cc21)N(c1ccc2c(c1)-c1ccccc1C2(c1ccccc1)c1ccccc1)c1ccccc1C31c2ccccc2-c2ccccc21 ; CC1(C)c2ccccc2-c2cc3c(cc21)N(c1ccc2c(c1)C(c1ccccc1)(c1ccccc1)c1cc4c(cc1-2)-c1ccccc1C4(C)C)c1ccccc1C31c2ccccc2-c2ccccc21] 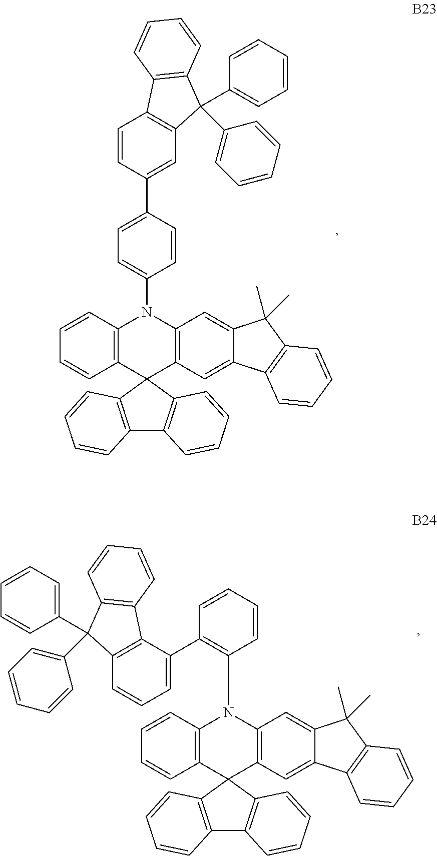 [SMILES: CC1(C)c2ccccc2-c2cc3c(cc21)N(c1ccc(-c2ccc4c(c2)C(c2ccccc2)(c2ccccc2)c2ccccc2-4)cc1)c1ccccc1C31c2ccccc2-c2ccccc21 ; CC1(C)c2ccccc2-c2cc3c(cc21)N(c1ccccc1-c1cccc2c1-c1ccccc1C2(c1ccccc1)c1ccccc1)c1ccccc1C31c2ccccc2-c2ccccc21] 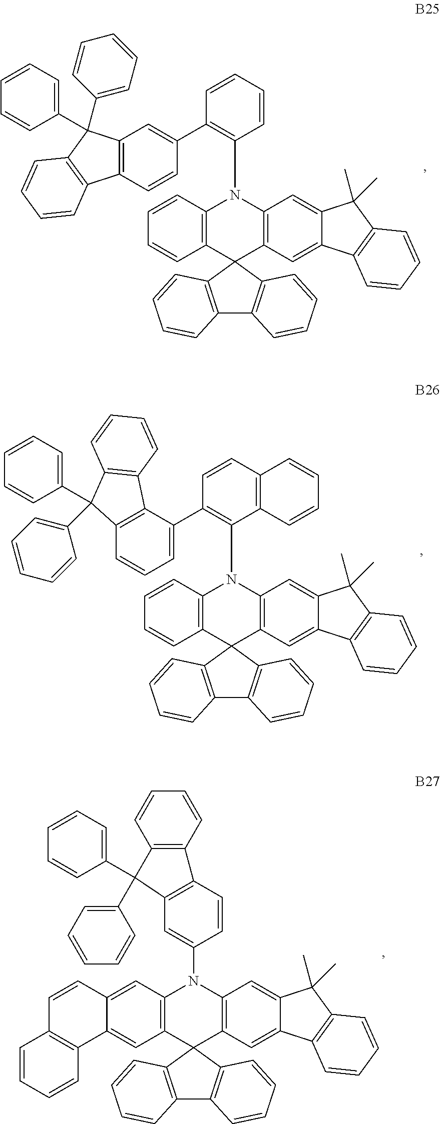 [SMILES: CC1(C)c2ccccc2-c2cc3c(cc21)N(c1ccccc1-c1ccc2c(c1)C(c1ccccc1)(c1ccccc1)c1ccccc1-2)c1ccccc1C31c2ccccc2-c2ccccc21 ; CC1(C)c2ccccc2-c2cc3c(cc21)N(c1c(-c2cccc4c2-c2ccccc2C4(c2ccccc2)c2ccccc2)ccc2ccccc12)c1ccccc1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2ccc4ccccc4c2cc1C31c2ccccc2-c2ccccc21] 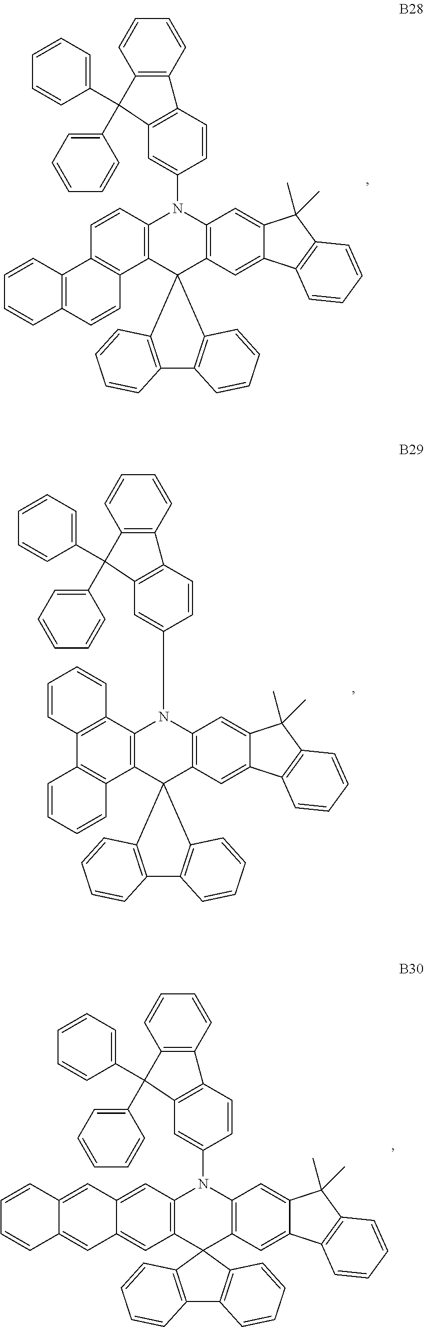 [SMILES: CC1(C)c2ccccc2-c2cc3c(cc21)N(c1ccc2c(c1)C(c1ccccc1)(c1ccccc1)c1ccccc1-2)c1ccc2c(ccc4ccccc42)c1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1c(c2ccccc2c2ccccc12)C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2cc4ccccc4cc2cc1C31c2ccccc2-c2ccccc21] 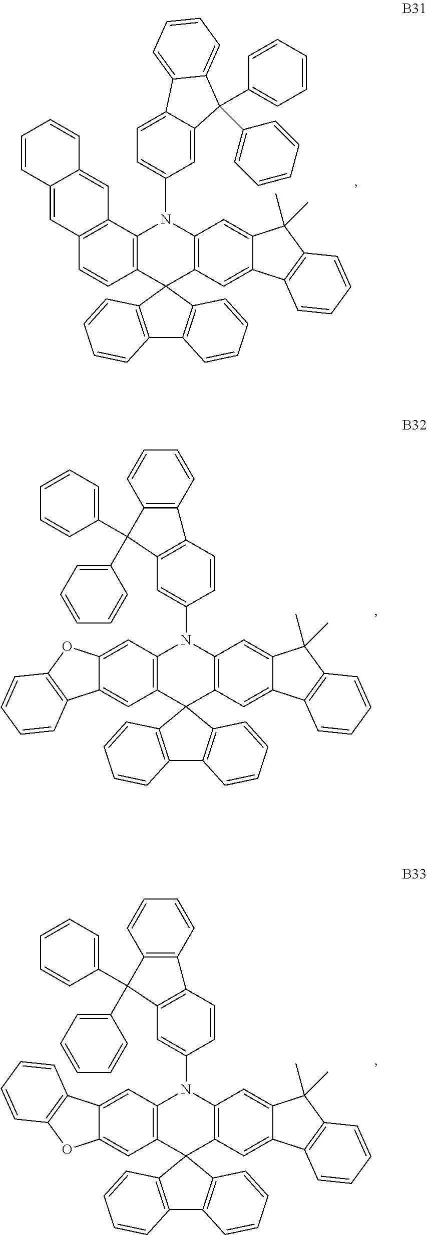 [SMILES: CC1(C)c2ccccc2-c2cc3c(cc21)N(c1ccc2c(c1)C(c1ccccc1)(c1ccccc1)c1ccccc1-2)c1c(ccc2cc4ccccc4cc12)C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2oc4ccccc4c2cc1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2c(cc1C31c3ccccc3-c3ccccc31)oc1ccccc12] 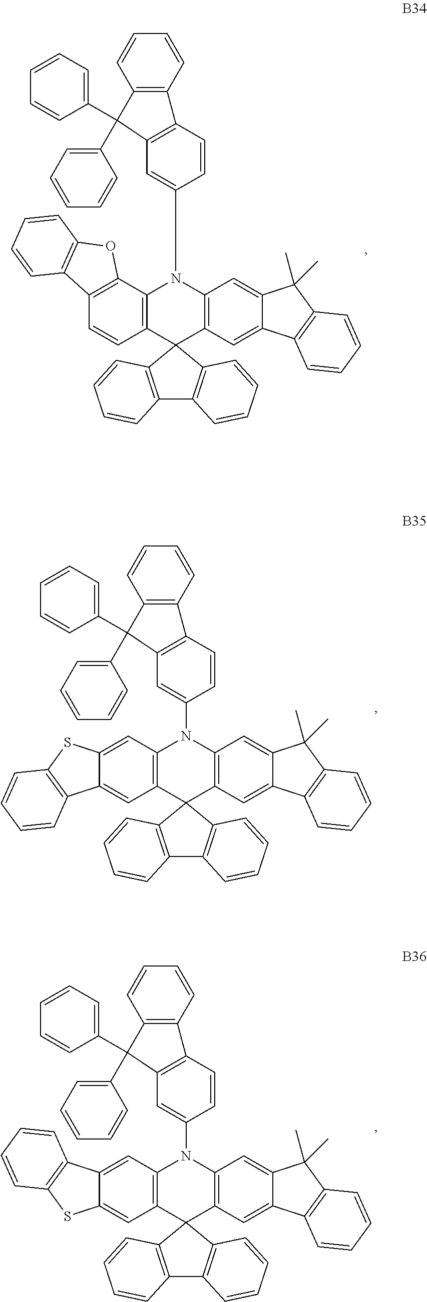 [SMILES: CC1(C)c2ccccc2-c2cc3c(cc21)N(c1ccc2c(c1)C(c1ccccc1)(c1ccccc1)c1ccccc1-2)c1c(ccc2c1oc1ccccc12)C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2sc4ccccc4c2cc1C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2c(cc1C31c3ccccc3-c3ccccc31)sc1ccccc12] 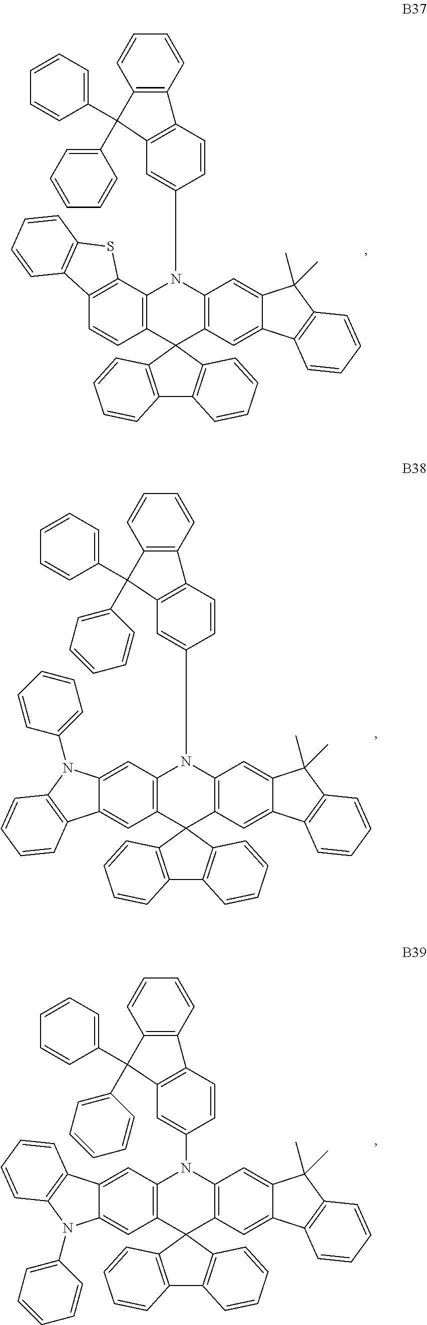 [SMILES: CC1(C)c2ccccc2-c2cc3c(cc21)N(c1ccc2c(c1)C(c1ccccc1)(c1ccccc1)c1ccccc1-2)c1c(ccc2c1sc1ccccc12)C31c2ccccc2-c2ccccc21 ; CC1(C)c2ccccc2-c2cc3c(cc21)N(c1ccc2c(c1)C(c1ccccc1)(c1ccccc1)c1ccccc1-2)c1cc2c(cc1C31c3ccccc3-c3ccccc31)c1ccccc1n2-c1ccccc1 ; CC1(C)c2ccccc2-c2cc3c(cc21)N(c1ccc2c(c1)C(c1ccccc1)(c1ccccc1)c1ccccc1-2)c1cc2c4ccccc4n(-c4ccccc4)c2cc1C31c2ccccc2-c2ccccc21] 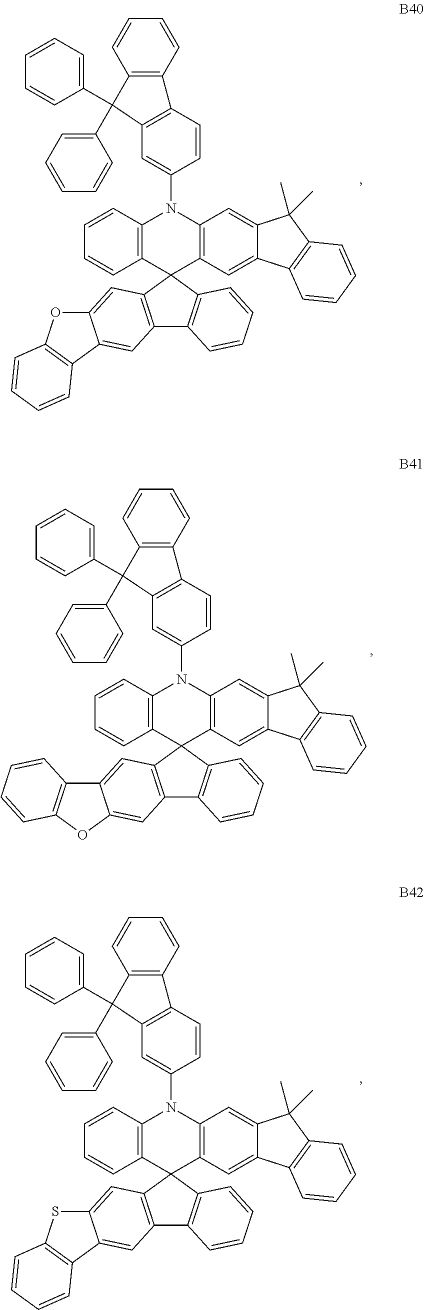 [SMILES: CC1(C)c2ccccc2-c2cc3c(cc21)N(c1ccc2c(c1)C(c1ccccc1)(c1ccccc1)c1ccccc1-2)c1ccccc1C31c2ccccc2-c2cc3c(cc21)oc1ccccc13 ; CC1(C)c2ccccc2-c2cc3c(cc21)N(c1ccc2c(c1)C(c1ccccc1)(c1ccccc1)c1ccccc1-2)c1ccccc1C31c2ccccc2-c2cc3oc4ccccc4c3cc21 ; CC1(C)c2ccccc2-c2cc3c(cc21)N(c1ccc2c(c1)C(c1ccccc1)(c1ccccc1)c1ccccc1-2)c1ccccc1C31c2ccccc2-c2cc3c(cc21)sc1ccccc13] 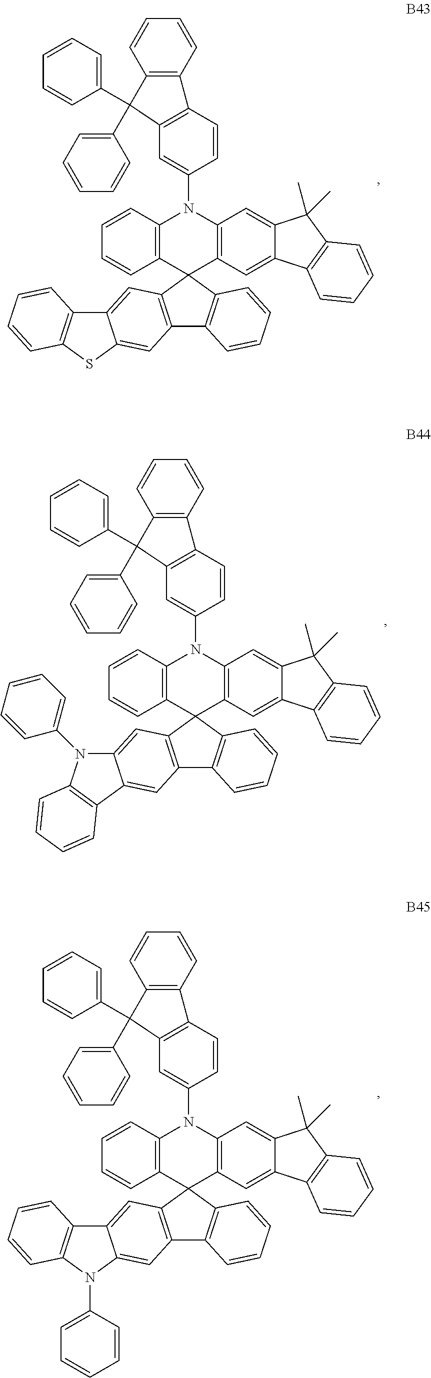 [SMILES: CC1(C)c2ccccc2-c2cc3c(cc21)N(c1ccc2c(c1)C(c1ccccc1)(c1ccccc1)c1ccccc1-2)c1ccccc1C31c2ccccc2-c2cc3sc4ccccc4c3cc21 ; CC1(C)c2ccccc2-c2cc3c(cc21)N(c1ccc2c(c1)C(c1ccccc1)(c1ccccc1)c1ccccc1-2)c1ccccc1C31c2ccccc2-c2cc3c4ccccc4n(-c4ccccc4)c3cc21 ; CC1(C)c2ccccc2-c2cc3c(cc21)N(c1ccc2c(c1)C(c1ccccc1)(c1ccccc1)c1ccccc1-2)c1ccccc1C31c2ccccc2-c2cc3c(cc21)c1ccccc1n3-c1ccccc1] 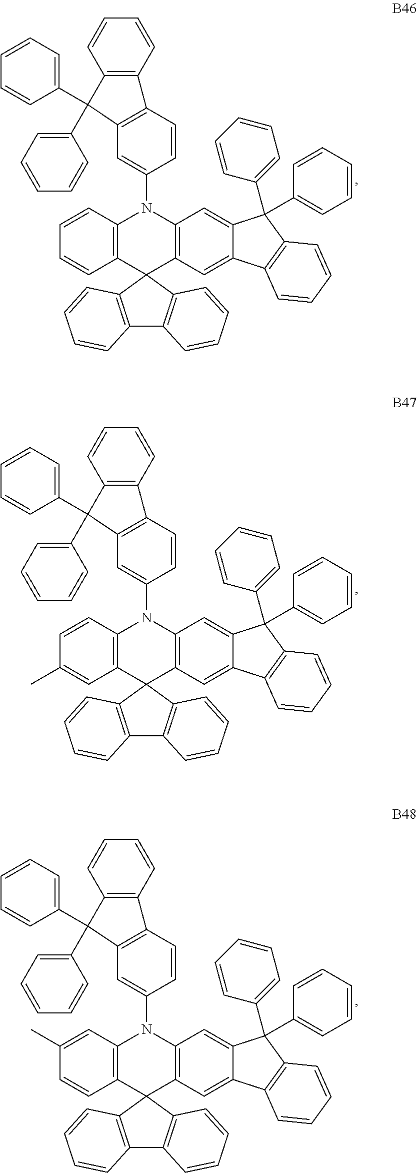 [SMILES: c1ccc(C2(c3ccccc3)c3ccccc3-c3ccc(N4c5ccccc5C5(c6ccccc6-c6ccccc65)c5cc6c(cc54)C(c4ccccc4)(c4ccccc4)c4ccccc4-6)cc32)cc1 ; Cc1ccc2c(c1)C1(c3ccccc3-c3ccccc31)c1cc3c(cc1N2c1ccc2c(c1)C(c1ccccc1)(c1ccccc1)c1ccccc1-2)C(c1ccccc1)(c1ccccc1)c1ccccc1-3 ; Cc1ccc2c(c1)N(c1ccc3c(c1)C(c1ccccc1)(c1ccccc1)c1ccccc1-3)c1cc3c(cc1C21c2ccccc2-c2ccccc21)-c1ccccc1C3(c1ccccc1)c1ccccc1] 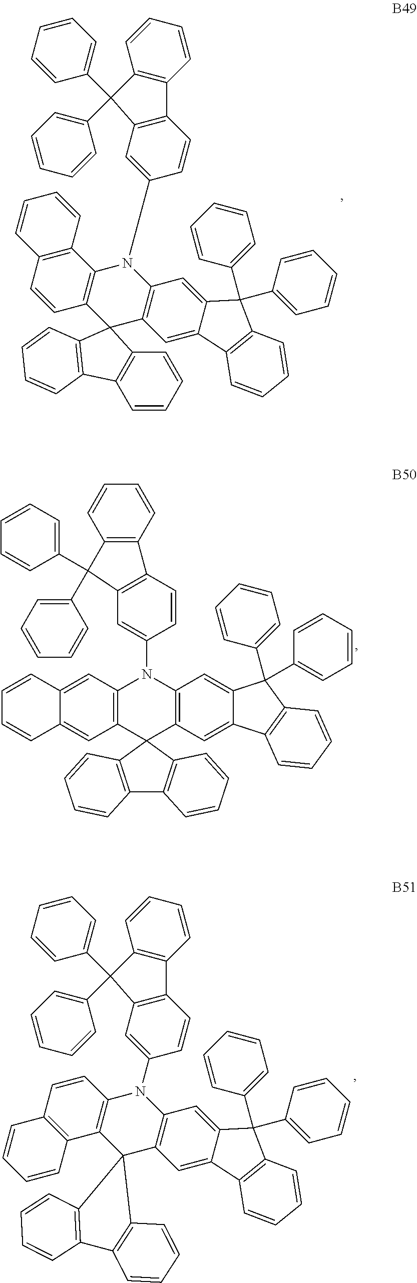 [SMILES: c1ccc(C2(c3ccccc3)c3ccccc3-c3ccc(N4c5cc6c(cc5C5(c7ccccc7-c7ccccc75)c5ccc7ccccc7c54)-c4ccccc4C6(c4ccccc4)c4ccccc4)cc32)cc1 ; c1ccc(C2(c3ccccc3)c3ccccc3-c3ccc(N4c5cc6c(cc5C5(c7ccccc7-c7ccccc75)c5cc7ccccc7cc54)-c4ccccc4C6(c4ccccc4)c4ccccc4)cc32)cc1 ; c1ccc(C2(c3ccccc3)c3ccccc3-c3ccc(N4c5cc6c(cc5C5(c7ccccc7-c7ccccc75)c5c4ccc4ccccc54)-c4ccccc4C6(c4ccccc4)c4ccccc4)cc32)cc1] 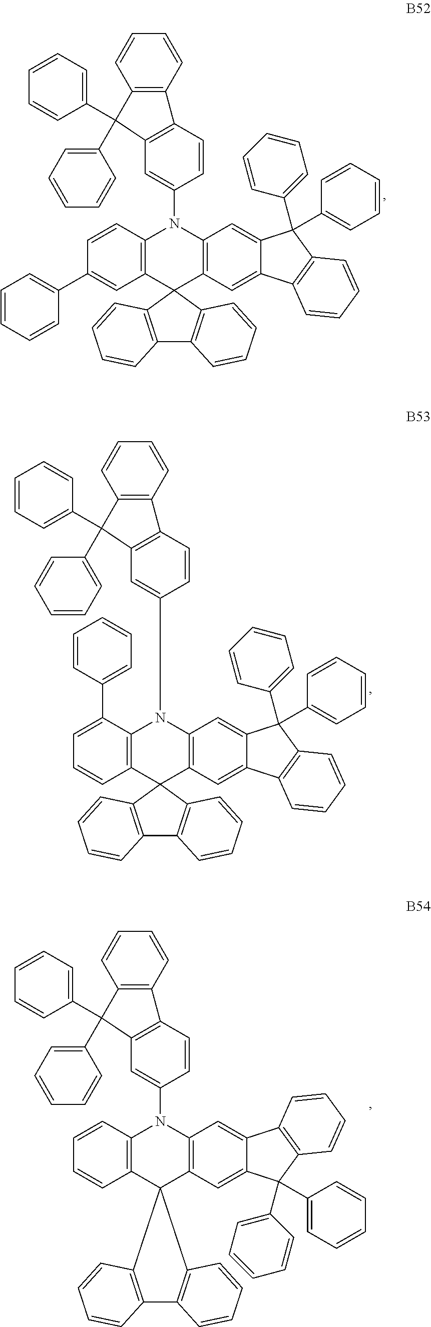 [SMILES: c1ccc(-c2ccc3c(c2)C2(c4ccccc4-c4ccccc42)c2cc4c(cc2N3c2ccc3c(c2)C(c2ccccc2)(c2ccccc2)c2ccccc2-3)C(c2ccccc2)(c2ccccc2)c2ccccc2-4)cc1 ; c1ccc(-c2cccc3c2N(c2ccc4c(c2)C(c2ccccc2)(c2ccccc2)c2ccccc2-4)c2cc4c(cc2C32c3ccccc3-c3ccccc32)-c2ccccc2C4(c2ccccc2)c2ccccc2)cc1 ; c1ccc(C2(c3ccccc3)c3ccccc3-c3ccc(N4c5ccccc5C5(c6ccccc6-c6ccccc65)c5cc6c(cc54)-c4ccccc4C6(c4ccccc4)c4ccccc4)cc32)cc1] 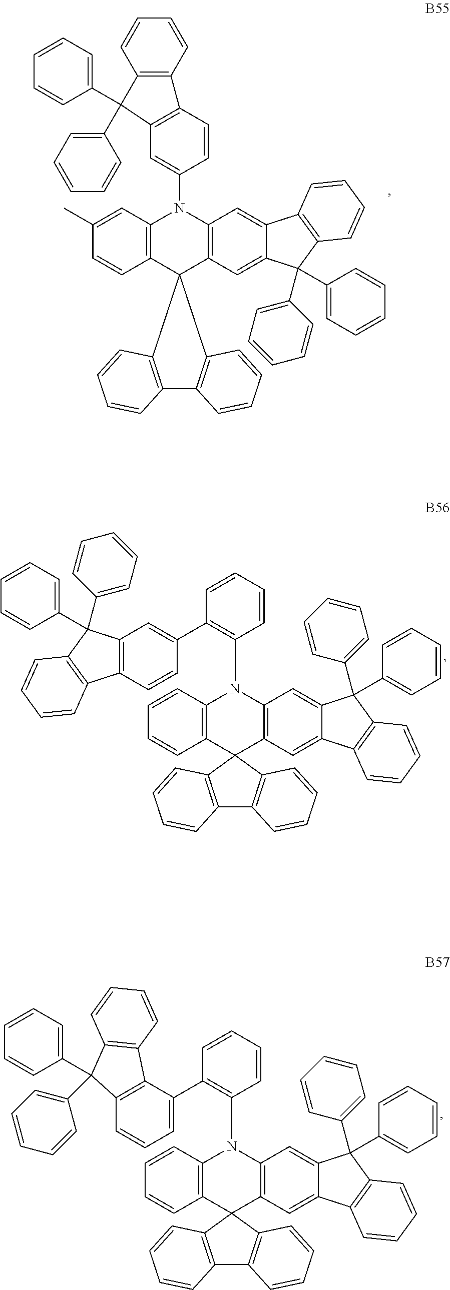 [SMILES: Cc1ccc2c(c1)N(c1ccc3c(c1)C(c1ccccc1)(c1ccccc1)c1ccccc1-3)c1cc3c(cc1C21c2ccccc2-c2ccccc21)C(c1ccccc1)(c1ccccc1)c1ccccc1-3 ; c1ccc(C2(c3ccccc3)c3ccccc3-c3ccc(-c4ccccc4N4c5ccccc5C5(c6ccccc6-c6ccccc65)c5cc6c(cc54)C(c4ccccc4)(c4ccccc4)c4ccccc4-6)cc32)cc1 ; c1ccc(C2(c3ccccc3)c3ccccc3-c3cc4c(cc32)N(c2ccccc2-c2cccc3c2-c2ccccc2C3(c2ccccc2)c2ccccc2)c2ccccc2C42c3ccccc3-c3ccccc32)cc1] 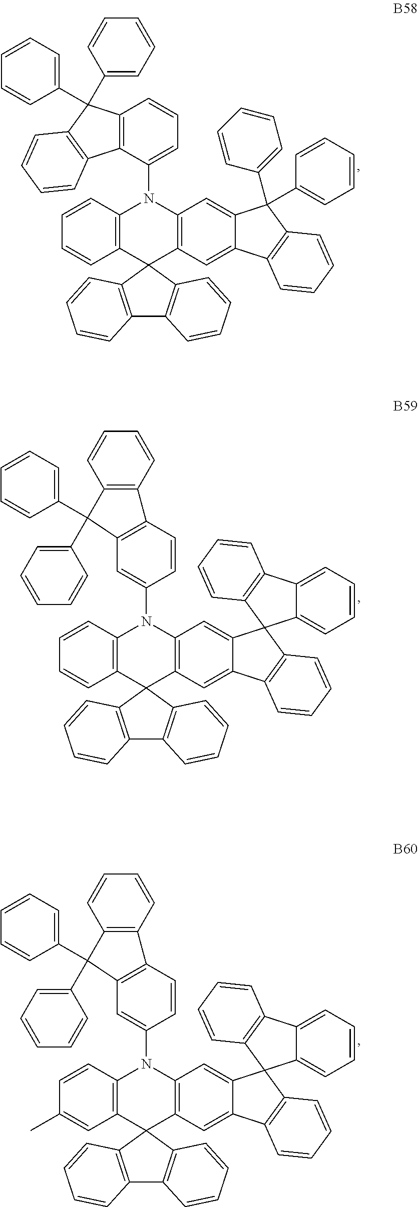 [SMILES: c1ccc(C2(c3ccccc3)c3ccccc3-c3cc4c(cc32)N(c2cccc3c2-c2ccccc2C3(c2ccccc2)c2ccccc2)c2ccccc2C42c3ccccc3-c3ccccc32)cc1 ; c1ccc(C2(c3ccccc3)c3ccccc3-c3ccc(N4c5ccccc5C5(c6ccccc6-c6ccccc65)c5cc6c(cc54)C4(c5ccccc5-c5ccccc54)c4ccccc4-6)cc32)cc1 ; Cc1ccc2c(c1)C1(c3ccccc3-c3ccccc31)c1cc3c(cc1N2c1ccc2c(c1)C(c1ccccc1)(c1ccccc1)c1ccccc1-2)C1(c2ccccc2-c2ccccc21)c1ccccc1-3] 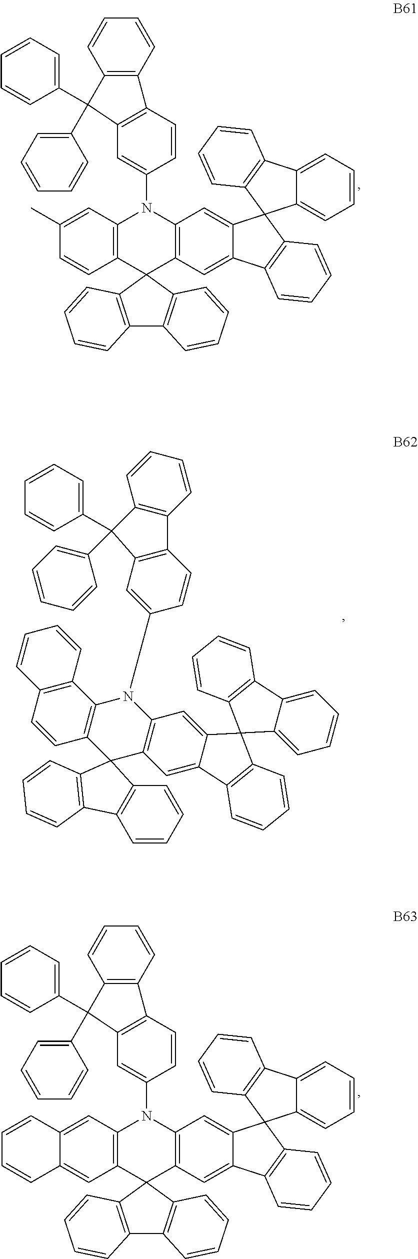 [SMILES: Cc1ccc2c(c1)N(c1ccc3c(c1)C(c1ccccc1)(c1ccccc1)c1ccccc1-3)c1cc3c(cc1C21c2ccccc2-c2ccccc21)-c1ccccc1C31c2ccccc2-c2ccccc21 ; c1ccc(C2(c3ccccc3)c3ccccc3-c3ccc(N4c5cc6c(cc5C5(c7ccccc7-c7ccccc75)c5ccc7ccccc7c54)-c4ccccc4C64c5ccccc5-c5ccccc54)cc32)cc1 ; c1ccc(C2(c3ccccc3)c3ccccc3-c3ccc(N4c5cc6c(cc5C5(c7ccccc7-c7ccccc75)c5cc7ccccc7cc54)-c4ccccc4C64c5ccccc5-c5ccccc54)cc32)cc1] 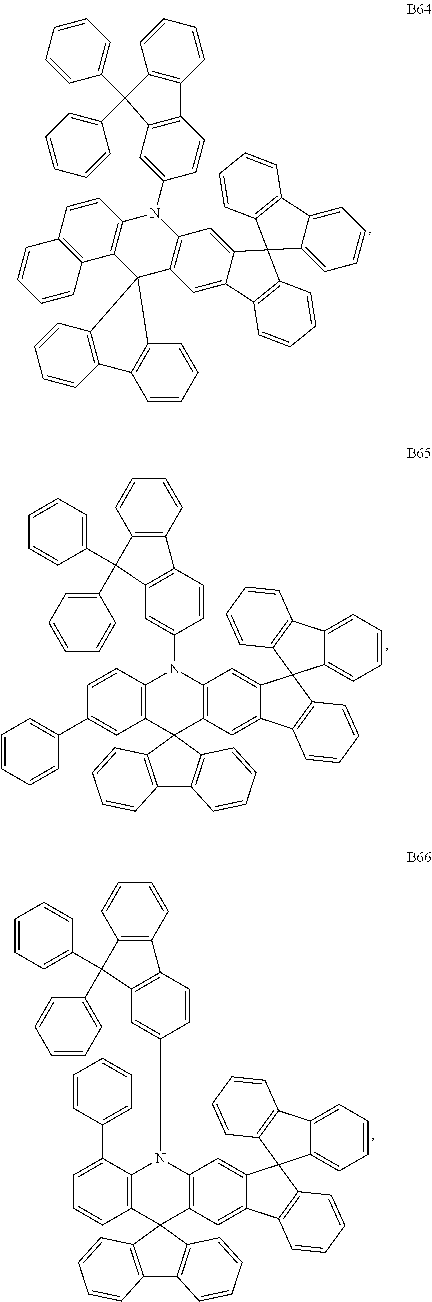 [SMILES: c1ccc(C2(c3ccccc3)c3ccccc3-c3ccc(N4c5cc6c(cc5C5(c7ccccc7-c7ccccc75)c5c4ccc4ccccc54)-c4ccccc4C64c5ccccc5-c5ccccc54)cc32)cc1 ; c1ccc(-c2ccc3c(c2)C2(c4ccccc4-c4ccccc42)c2cc4c(cc2N3c2ccc3c(c2)C(c2ccccc2)(c2ccccc2)c2ccccc2-3)C2(c3ccccc3-c3ccccc32)c2ccccc2-4)cc1 ; c1ccc(-c2cccc3c2N(c2ccc4c(c2)C(c2ccccc2)(c2ccccc2)c2ccccc2-4)c2cc4c(cc2C32c3ccccc3-c3ccccc32)-c2ccccc2C42c3ccccc3-c3ccccc32)cc1] 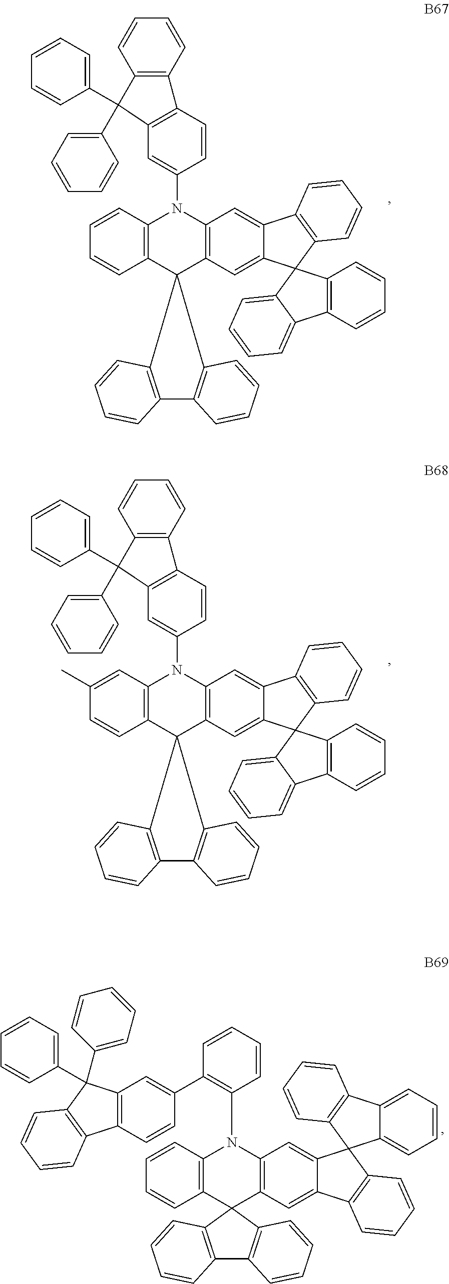 [SMILES: c1ccc(C2(c3ccccc3)c3ccccc3-c3ccc(N4c5ccccc5C5(c6ccccc6-c6ccccc65)c5cc6c(cc54)-c4ccccc4C64c5ccccc5-c5ccccc54)cc32)cc1 ; Cc1ccc2c(c1)N(c1ccc3c(c1)C(c1ccccc1)(c1ccccc1)c1ccccc1-3)c1cc3c(cc1C21c2ccccc2-c2ccccc21)C1(c2ccccc2-c2ccccc21)c1ccccc1-3 ; c1ccc(C2(c3ccccc3)c3ccccc3-c3ccc(-c4ccccc4N4c5ccccc5C5(c6ccccc6-c6ccccc65)c5cc6c(cc54)C4(c5ccccc5-c5ccccc54)c4ccccc4-6)cc32)cc1] 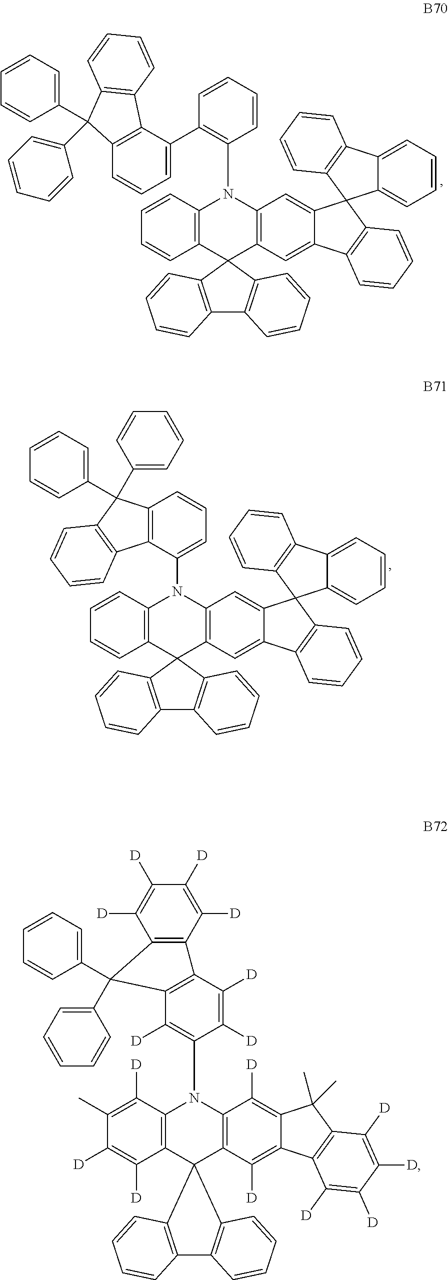 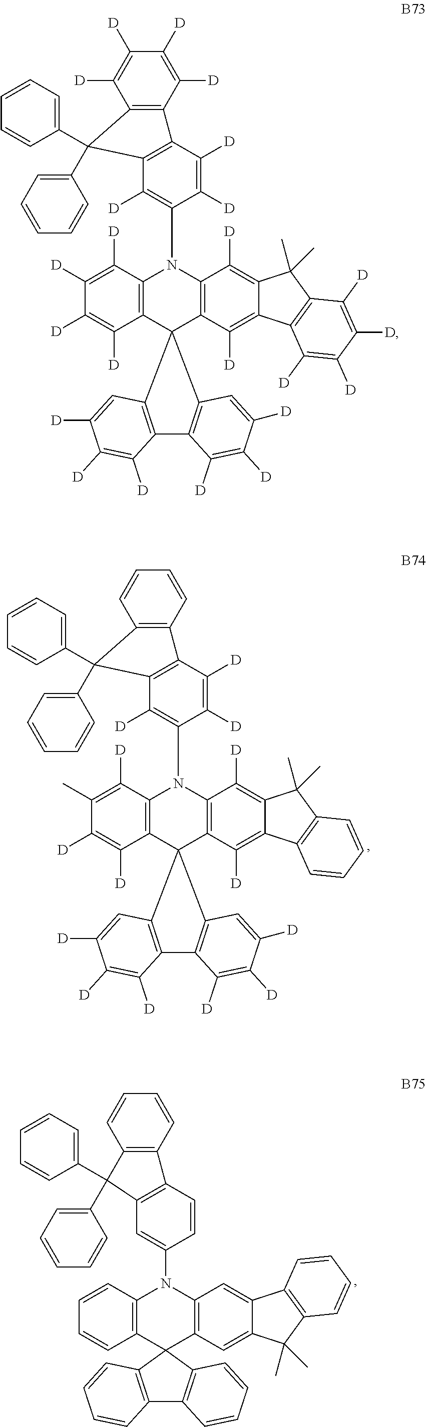 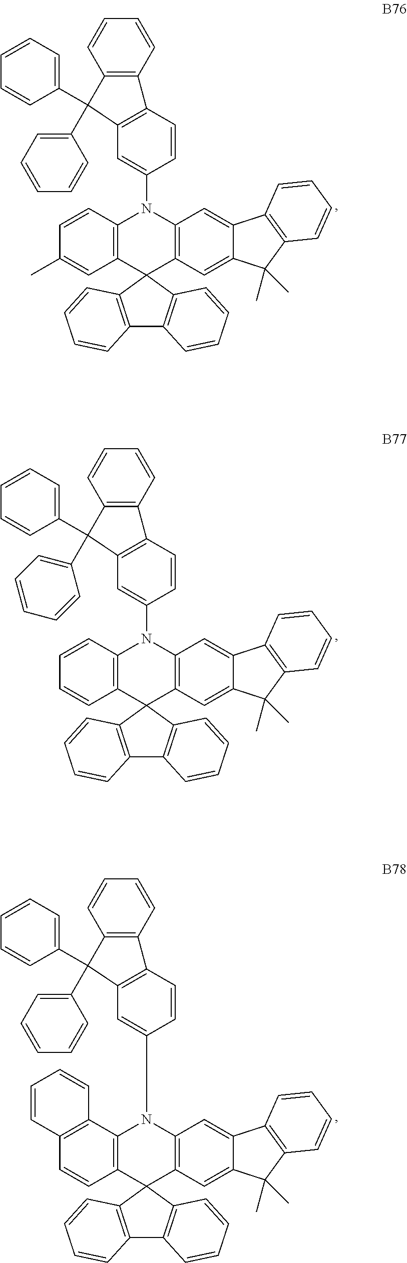 [SMILES: Cc1ccc2c(c1)C1(c3ccccc3-c3ccccc31)c1cc3c(cc1N2c1ccc2c(c1)C(c1ccccc1)(c1ccccc1)c1ccccc1-2)-c1ccccc1C3(C)C ; CC1(C)c2ccccc2-c2cc3c(cc21)C1(c2ccccc2-c2ccccc21)c1ccccc1N3c1ccc2c(c1)C(c1ccccc1)(c1ccccc1)c1ccccc1-2 ; CC1(C)c2ccccc2-c2cc3c(cc21)C1(c2ccccc2-c2ccccc21)c1ccc2ccccc2c1N3c1ccc2c(c1)C(c1ccccc1)(c1ccccc1)c1ccccc1-2] 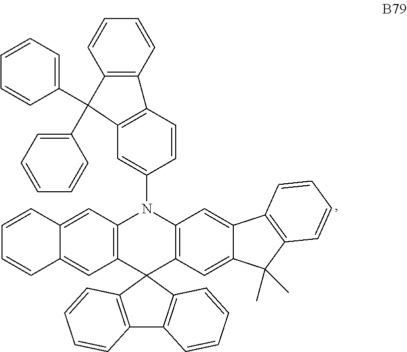 [SMILES: CC1(C)c2ccccc2-c2cc3c(cc21)C1(c2ccccc2-c2ccccc21)c1cc2ccccc2cc1N3c1ccc2c(c1)C(c1ccccc1)(c1ccccc1)c1ccccc1-2] 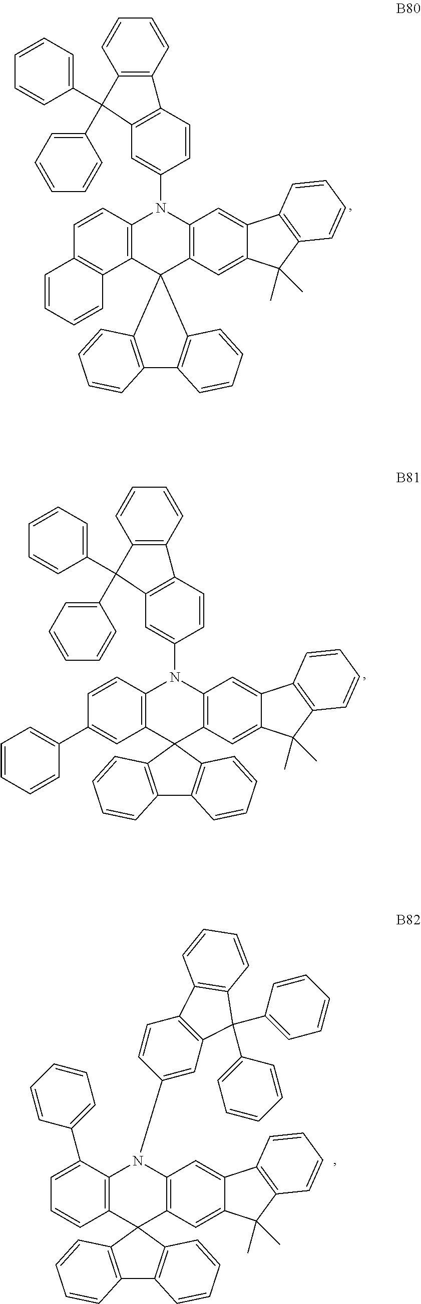 [SMILES: CC1(C)c2ccccc2-c2cc3c(cc21)C1(c2ccccc2-c2ccccc21)c1c(ccc2ccccc12)N3c1ccc2c(c1)C(c1ccccc1)(c1ccccc1)c1ccccc1-2 ; CC1(C)c2ccccc2-c2cc3c(cc21)C1(c2ccccc2-c2ccccc21)c1cc(-c2ccccc2)ccc1N3c1ccc2c(c1)C(c1ccccc1)(c1ccccc1)c1ccccc1-2 ; CC1(C)c2ccccc2-c2cc3c(cc21)C1(c2ccccc2-c2ccccc21)c1cccc(-c2ccccc2)c1N3c1ccc2c(c1)C(c1ccccc1)(c1ccccc1)c1ccccc1-2] 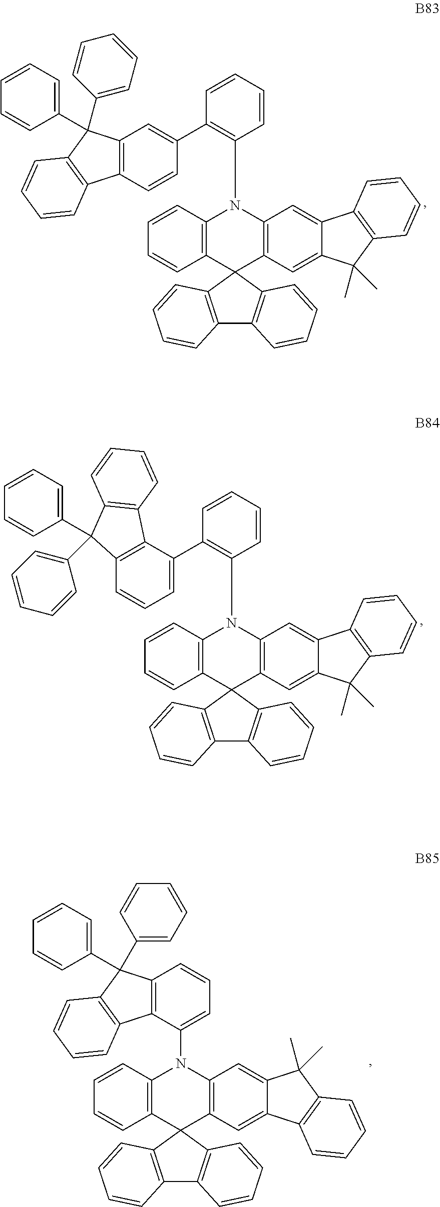 [SMILES: CC1(C)c2ccccc2-c2cc3c(cc21)C1(c2ccccc2-c2ccccc21)c1ccccc1N3c1ccccc1-c1ccc2c(c1)C(c1ccccc1)(c1ccccc1)c1ccccc1-2 ; CC1(C)c2ccccc2-c2cc3c(cc21)C1(c2ccccc2-c2ccccc21)c1ccccc1N3c1ccccc1-c1cccc2c1-c1ccccc1C2(c1ccccc1)c1ccccc1 ; CC1(C)c2ccccc2-c2cc3c(cc21)N(c1cccc2c1-c1ccccc1C2(c1ccccc1)c1ccccc1)c1ccccc1C31c2ccccc2-c2ccccc21] 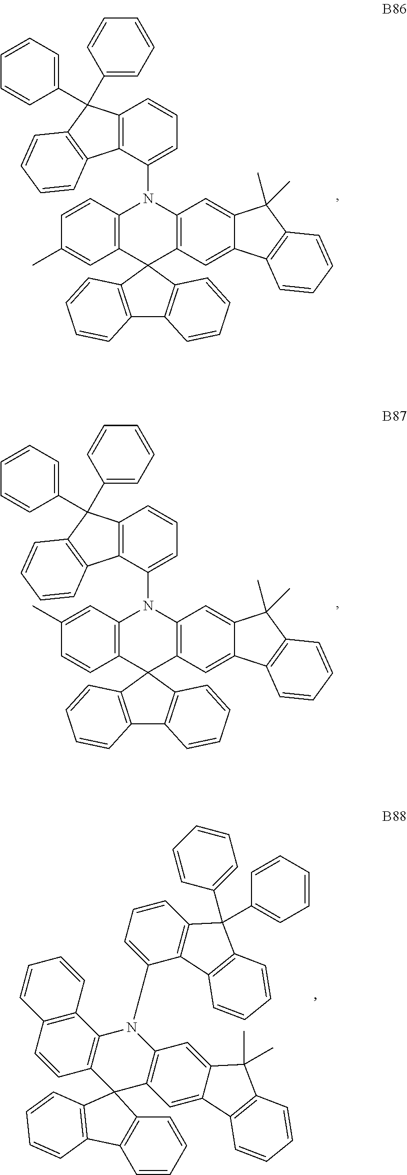 [SMILES: Cc1ccc2c(c1)C1(c3ccccc3-c3ccccc31)c1cc3c(cc1N2c1cccc2c1-c1ccccc1C2(c1ccccc1)c1ccccc1)C(C)(C)c1ccccc1-3 ; Cc1ccc2c(c1)N(c1cccc3c1-c1ccccc1C3(c1ccccc1)c1ccccc1)c1cc3c(cc1C21c2ccccc2-c2ccccc21)-c1ccccc1C3(C)C ; CC1(C)c2ccccc2-c2cc3c(cc21)N(c1cccc2c1-c1ccccc1C2(c1ccccc1)c1ccccc1)c1c(ccc2ccccc12)C31c2ccccc2-c2ccccc21] 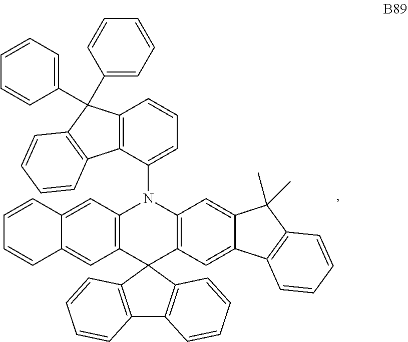 [SMILES: CC1(C)c2ccccc2-c2cc3c(cc21)N(c1cccc2c1-c1ccccc1C2(c1ccccc1)c1ccccc1)c1cc2ccccc2cc1C31c2ccccc2-c2ccccc21] 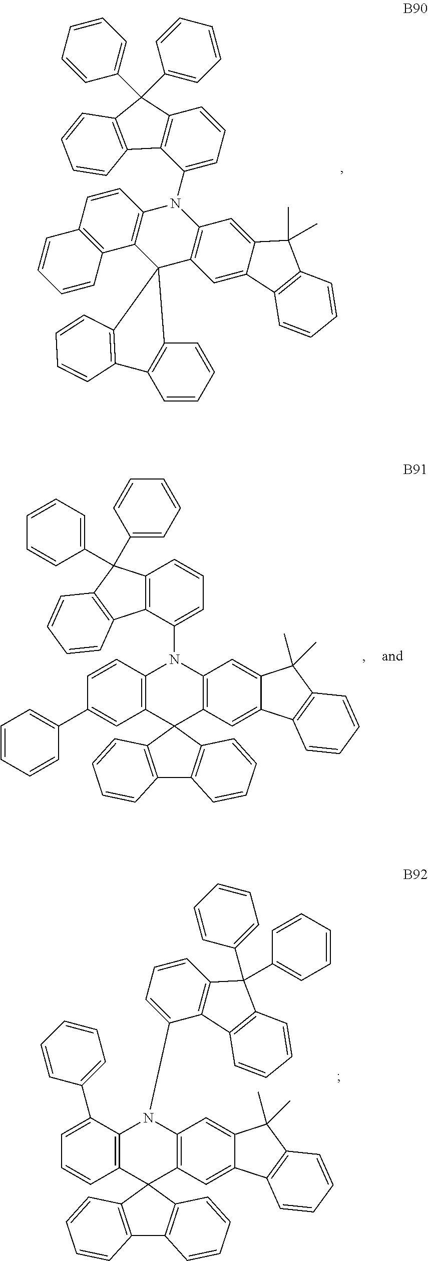 [SMILES: CC1(C)c2ccccc2-c2cc3c(cc21)N(c1cccc2c1-c1ccccc1C2(c1ccccc1)c1ccccc1)c1ccc2ccccc2c1C31c2ccccc2-c2ccccc21 ; CC1(C)c2ccccc2-c2cc3c(cc21)N(c1cccc2c1-c1ccccc1C2(c1ccccc1)c1ccccc1)c1ccc(-c2ccccc2)cc1C31c2ccccc2-c2ccccc21 ; CC1(C)c2ccccc2-c2cc3c(cc21)N(c1cccc2c1-c1ccccc1C2(c1ccccc1)c1ccccc1)c1c(-c2ccccc2)cccc1C31c2ccccc2-c2ccccc21] 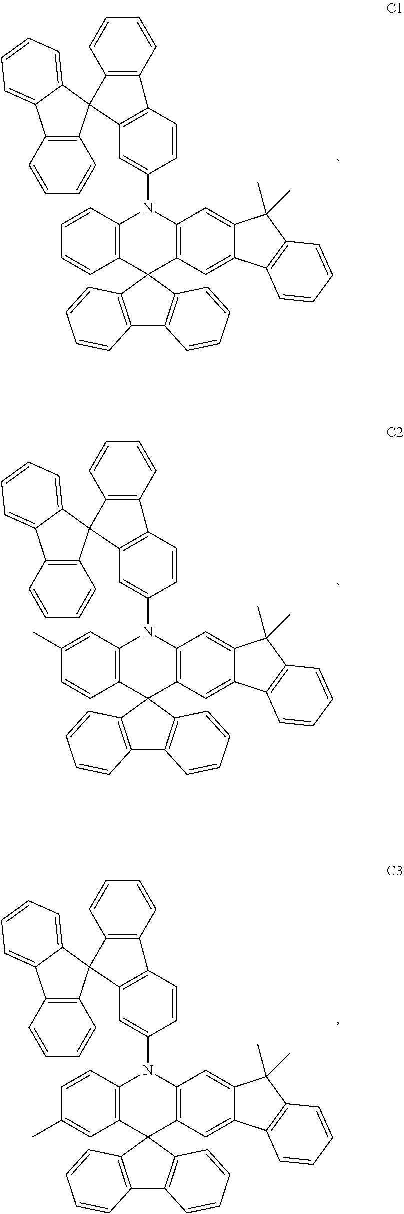 [SMILES: CC1(C)c2ccccc2-c2cc3c(cc21)N(c1ccc2c(c1)C1(c4ccccc4-c4ccccc41)c1ccccc1-2)c1ccccc1C31c2ccccc2-c2ccccc21 ; Cc1ccc2c(c1)N(c1ccc3c(c1)C1(c4ccccc4-c4ccccc41)c1ccccc1-3)c1cc3c(cc1C21c2ccccc2-c2ccccc21)-c1ccccc1C3(C)C ; Cc1ccc2c(c1)C1(c3ccccc3-c3ccccc31)c1cc3c(cc1N2c1ccc2c(c1)C1(c4ccccc4-c4ccccc41)c1ccccc1-2)C(C)(C)c1ccccc1-3] 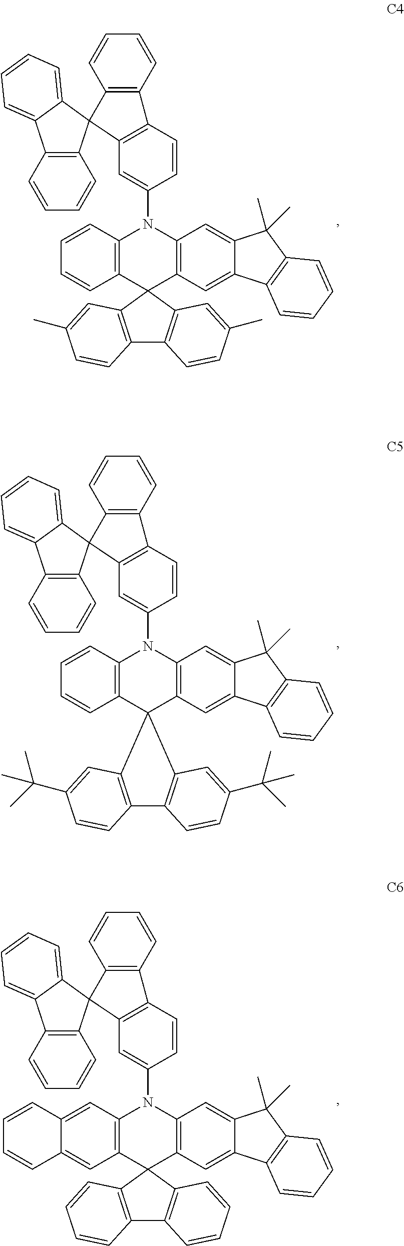 [SMILES: Cc1ccc2c(c1)C1(c3cc(C)ccc3-2)c2ccccc2N(c2ccc3c(c2)C2(c4ccccc4-c4ccccc42)c2ccccc2-3)c2cc3c(cc21)-c1ccccc1C3(C)C ; CC(C)(C)c1ccc2c(c1)C1(c3cc(C(C)(C)C)ccc3-2)c2ccccc2N(c2ccc3c(c2)C2(c4ccccc4-c4ccccc42)c2ccccc2-3)c2cc3c(cc21)-c1ccccc1C3(C)C ; CC1(C)c2ccccc2-c2cc3c(cc21)N(c1ccc2c(c1)C1(c4ccccc4-c4ccccc41)c1ccccc1-2)c1cc2ccccc2cc1C31c2ccccc2-c2ccccc21] 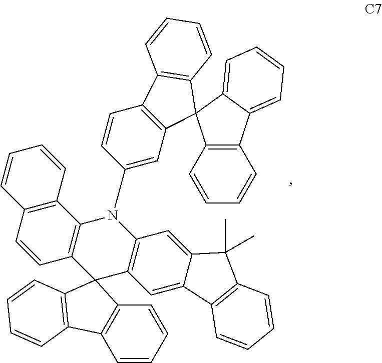 [SMILES: CC1(C)c2ccccc2-c2cc3c(cc21)N(c1ccc2c(c1)C1(c4ccccc4-c4ccccc41)c1ccccc1-2)c1c(ccc2ccccc12)C31c2ccccc2-c2ccccc21] 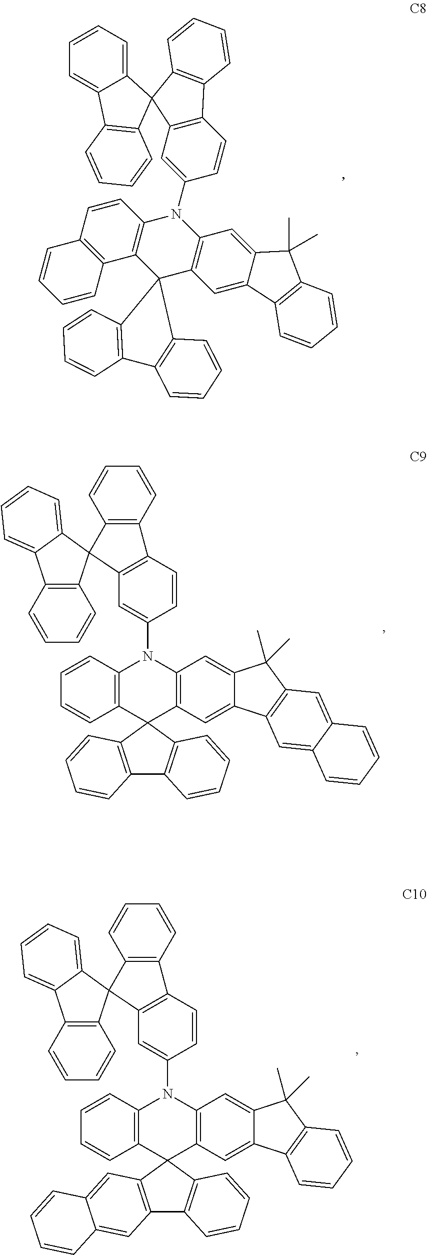 [SMILES: CC1(C)c2ccccc2-c2cc3c(cc21)N(c1ccc2c(c1)C1(c4ccccc4-c4ccccc41)c1ccccc1-2)c1ccc2ccccc2c1C31c2ccccc2-c2ccccc21 ; CC1(C)c2cc3c(cc2-c2cc4ccccc4cc21)C1(c2ccccc2-c2ccccc21)c1ccccc1N3c1ccc2c(c1)C1(c3ccccc3-c3ccccc31)c1ccccc1-2 ; CC1(C)c2ccccc2-c2cc3c(cc21)N(c1ccc2c(c1)C1(c4ccccc4-c4ccccc41)c1ccccc1-2)c1ccccc1C31c2ccccc2-c2cc3ccccc3cc21] 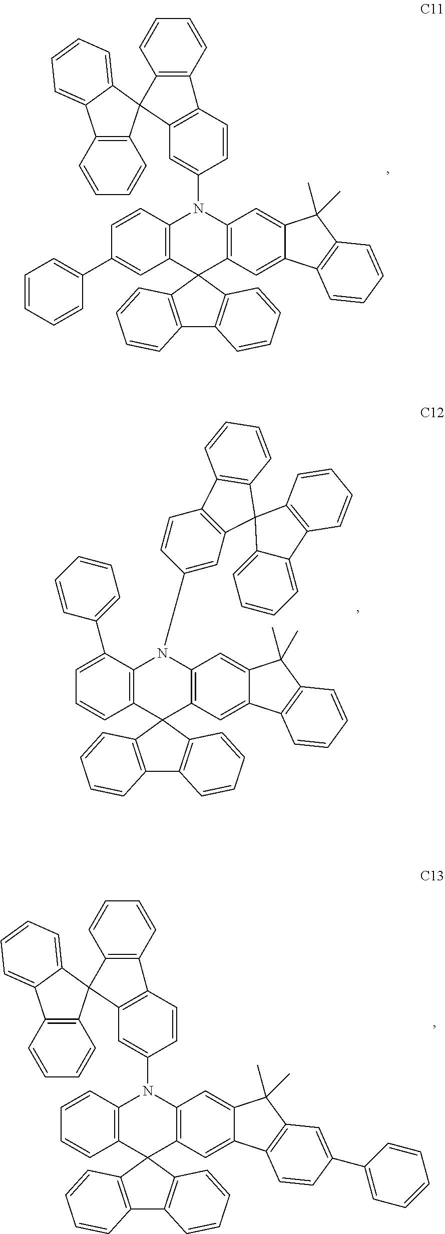 [SMILES: CC1(C)c2ccccc2-c2cc3c(cc21)N(c1ccc2c(c1)C1(c4ccccc4-c4ccccc41)c1ccccc1-2)c1ccc(-c2ccccc2)cc1C31c2ccccc2-c2ccccc21 ; CC1(C)c2ccccc2-c2cc3c(cc21)N(c1ccc2c(c1)C1(c4ccccc4-c4ccccc41)c1ccccc1-2)c1c(-c2ccccc2)cccc1C31c2ccccc2-c2ccccc21 ; CC1(C)c2cc(-c3ccccc3)ccc2-c2cc3c(cc21)N(c1ccc2c(c1)C1(c4ccccc4-c4ccccc41)c1ccccc1-2)c1ccccc1C31c2ccccc2-c2ccccc21] 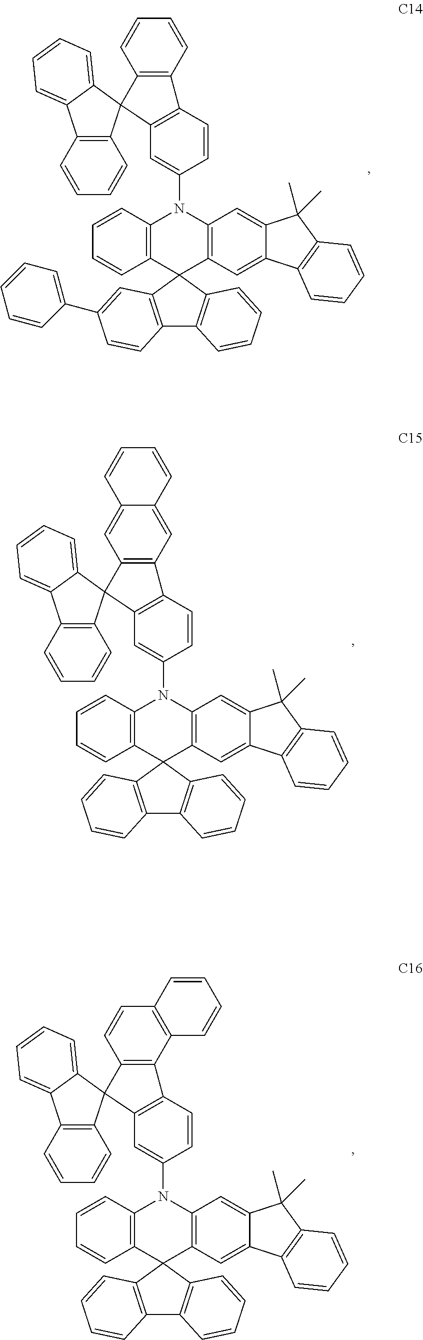 [SMILES: CC1(C)c2ccccc2-c2cc3c(cc21)N(c1ccc2c(c1)C1(c4ccccc4-c4ccccc41)c1ccccc1-2)c1ccccc1C31c2ccccc2-c2ccc(-c3ccccc3)cc21 ; CC1(C)c2ccccc2-c2cc3c(cc21)N(c1ccc2c(c1)C1(c4ccccc4-c4ccccc41)c1cc4ccccc4cc1-2)c1ccccc1C31c2ccccc2-c2ccccc21 ; CC1(C)c2ccccc2-c2cc3c(cc21)N(c1ccc2c(c1)C1(c4ccccc4-c4ccccc41)c1ccc4ccccc4c1-2)c1ccccc1C31c2ccccc2-c2ccccc21] 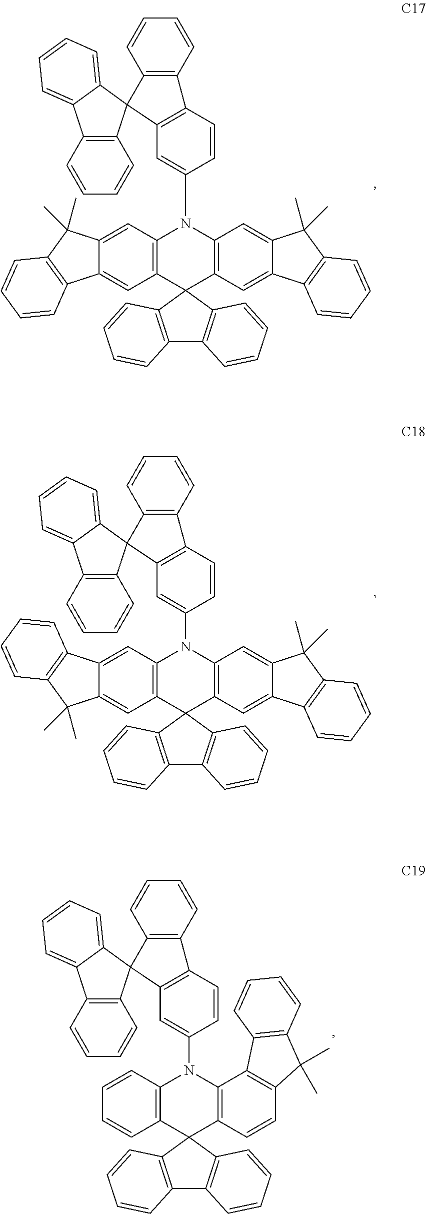 [SMILES: CC1(C)c2ccccc2-c2cc3c(cc21)N(c1ccc2c(c1)C1(c4ccccc4-c4ccccc41)c1ccccc1-2)c1cc2c(cc1C31c3ccccc3-c3ccccc31)-c1ccccc1C2(C)C ; CC1(C)c2ccccc2-c2cc3c(cc21)N(c1ccc2c(c1)C1(c4ccccc4-c4ccccc41)c1ccccc1-2)c1cc2c(cc1C31c3ccccc3-c3ccccc31)C(C)(C)c1ccccc1-2 ; CC1(C)c2ccccc2-c2c1ccc1c2N(c2ccc3c(c2)C2(c4ccccc4-c4ccccc42)c2ccccc2-3)c2ccccc2C12c1ccccc1-c1ccccc12] 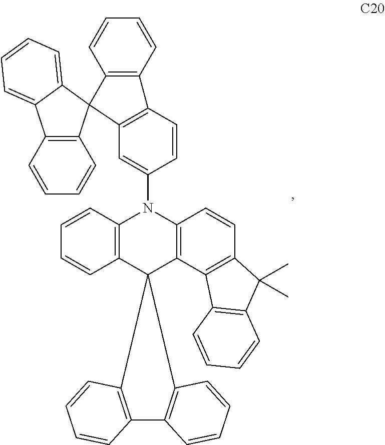 [SMILES: CC1(C)c2ccccc2-c2c1ccc1c2C2(c3ccccc3-c3ccccc32)c2ccccc2N1c1ccc2c(c1)C1(c3ccccc3-c3ccccc31)c1ccccc1-2] 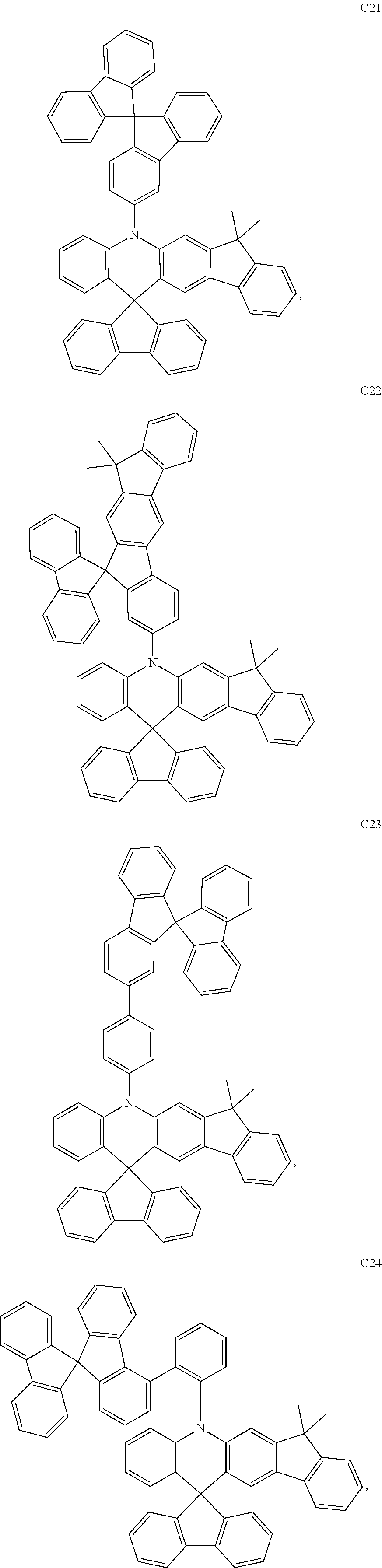 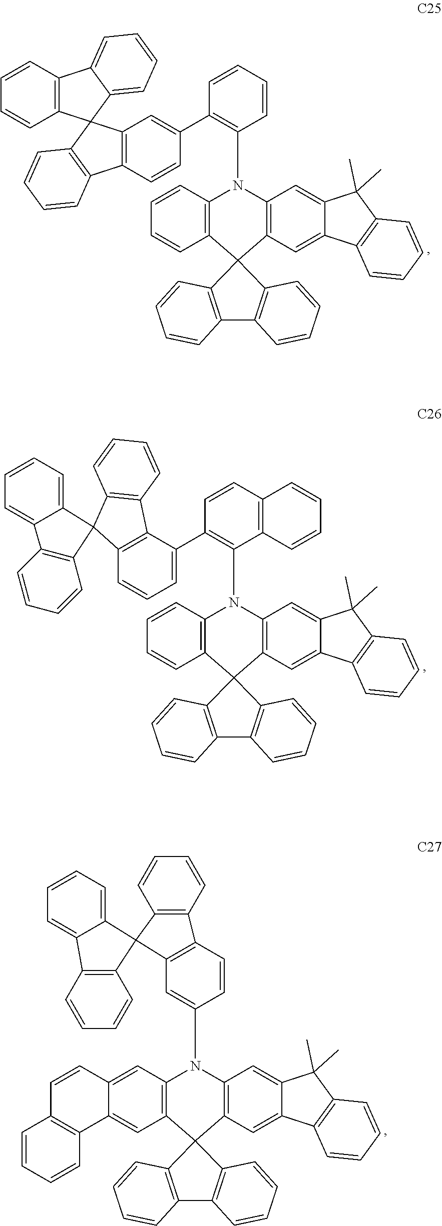 [SMILES: CC1(C)c2ccccc2-c2cc3c(cc21)N(c1ccccc1-c1ccc2c(c1)C1(c4ccccc4-c4ccccc41)c1ccccc1-2)c1ccccc1C31c2ccccc2-c2ccccc21 ; CC1(C)c2ccccc2-c2cc3c(cc21)N(c1c(-c2cccc4c2-c2ccccc2C42c4ccccc4-c4ccccc42)ccc2ccccc12)c1ccccc1C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2ccc4ccccc4c2cc1C31c2ccccc2-c2ccccc21] 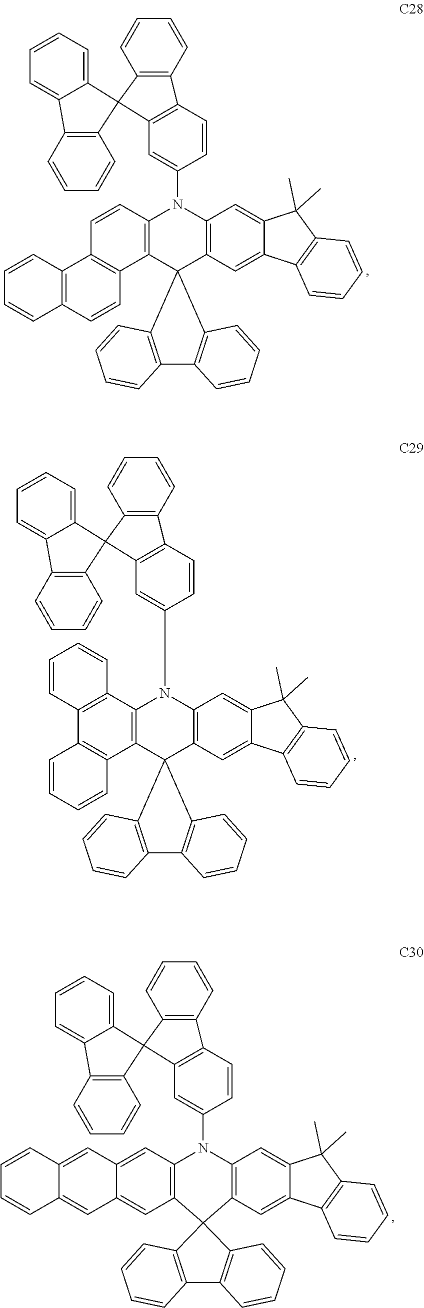 [SMILES: CC1(C)c2ccccc2-c2cc3c(cc21)N(c1ccc2c(c1)C1(c4ccccc4-c4ccccc41)c1ccccc1-2)c1ccc2c(ccc4ccccc42)c1C31c2ccccc2-c2ccccc21 ; CC1(C)c2ccccc2-c2cc3c(cc21)N(c1ccc2c(c1)C1(c4ccccc4-c4ccccc41)c1ccccc1-2)c1c(c2ccccc2c2ccccc12)C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2cc4ccccc4cc2cc1C31c2ccccc2-c2ccccc21] 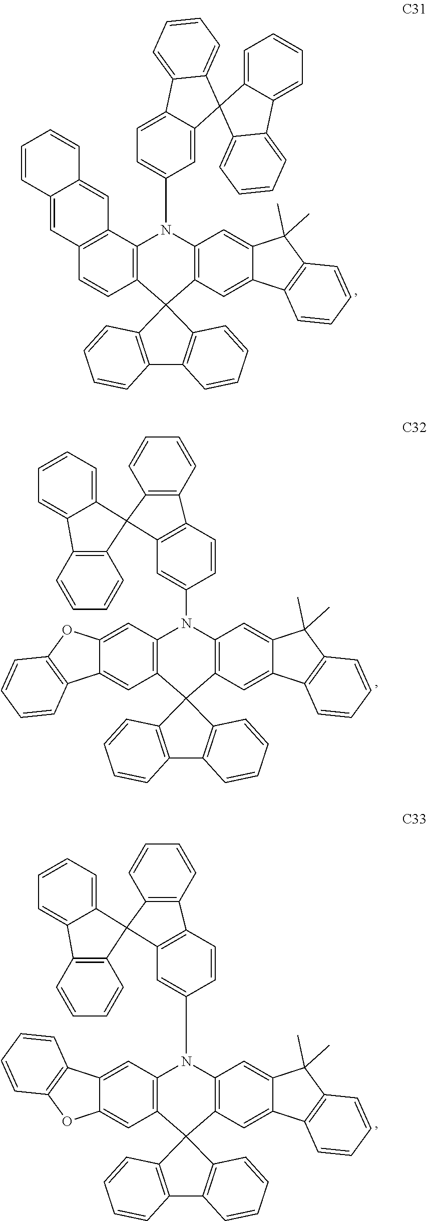 [SMILES: CC1(C)c2ccccc2-c2cc3c(cc21)N(c1ccc2c(c1)C1(c4ccccc4-c4ccccc41)c1ccccc1-2)c1c(ccc2cc4ccccc4cc12)C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2oc4ccccc4c2cc1C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2c(cc1C31c3ccccc3-c3ccccc31)oc1ccccc12] 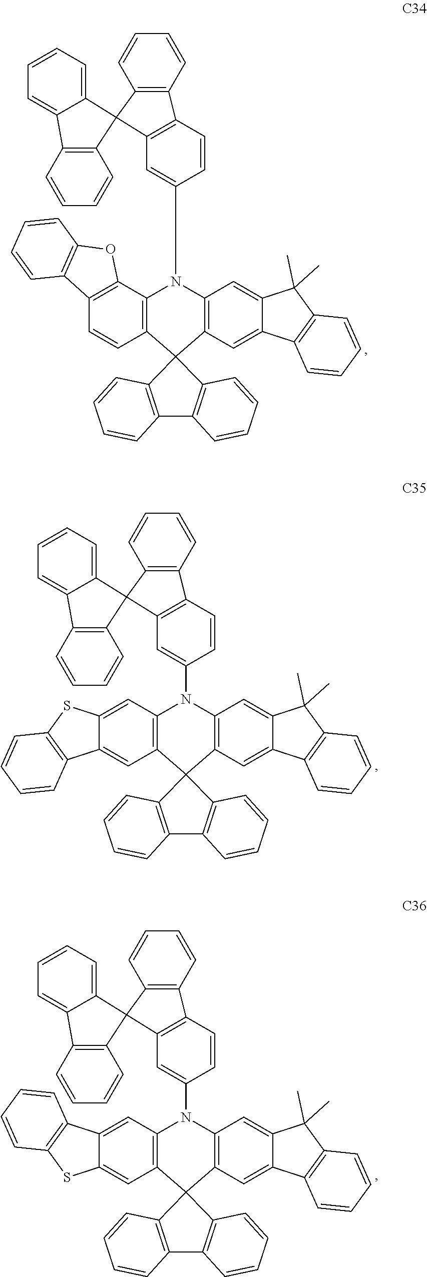 [SMILES: CC1(C)c2ccccc2-c2cc3c(cc21)N(c1ccc2c(c1)C1(c4ccccc4-c4ccccc41)c1ccccc1-2)c1c(ccc2c1oc1ccccc12)C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2sc4ccccc4c2cc1C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2c(cc1C31c3ccccc3-c3ccccc31)sc1ccccc12] 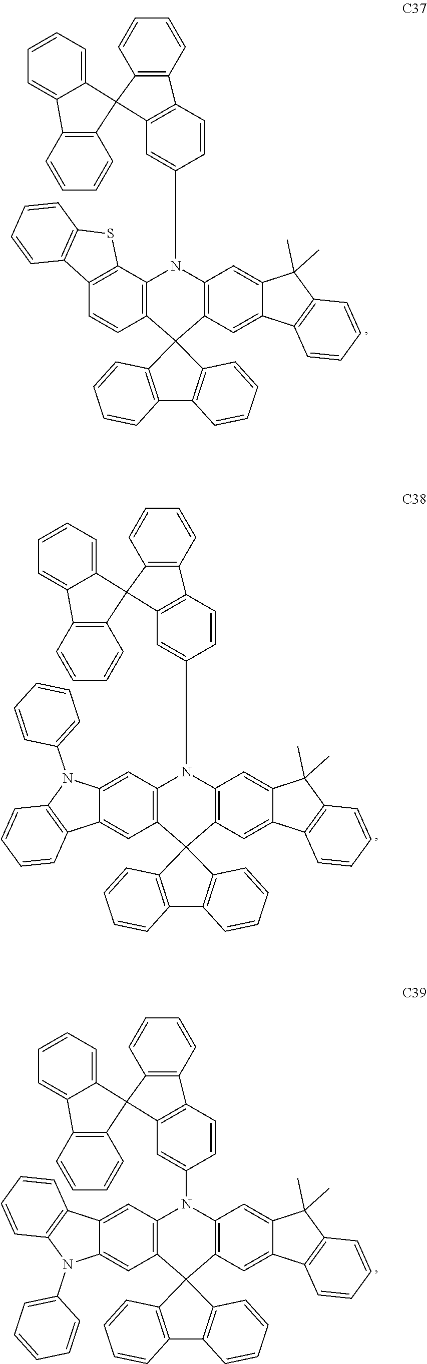 [SMILES: CC1(C)c2ccccc2-c2cc3c(cc21)N(c1ccc2c(c1)C1(c4ccccc4-c4ccccc41)c1ccccc1-2)c1c(ccc2c1sc1ccccc12)C31c2ccccc2-c2ccccc21 ; CC1(C)c2ccccc2-c2cc3c(cc21)N(c1ccc2c(c1)C1(c4ccccc4-c4ccccc41)c1ccccc1-2)c1cc2c(cc1C31c3ccccc3-c3ccccc31)c1ccccc1n2-c1ccccc1 ; CC1(C)c2ccccc2-c2cc3c(cc21)N(c1ccc2c(c1)C1(c4ccccc4-c4ccccc41)c1ccccc1-2)c1cc2c4ccccc4n(-c4ccccc4)c2cc1C31c2ccccc2-c2ccccc21] 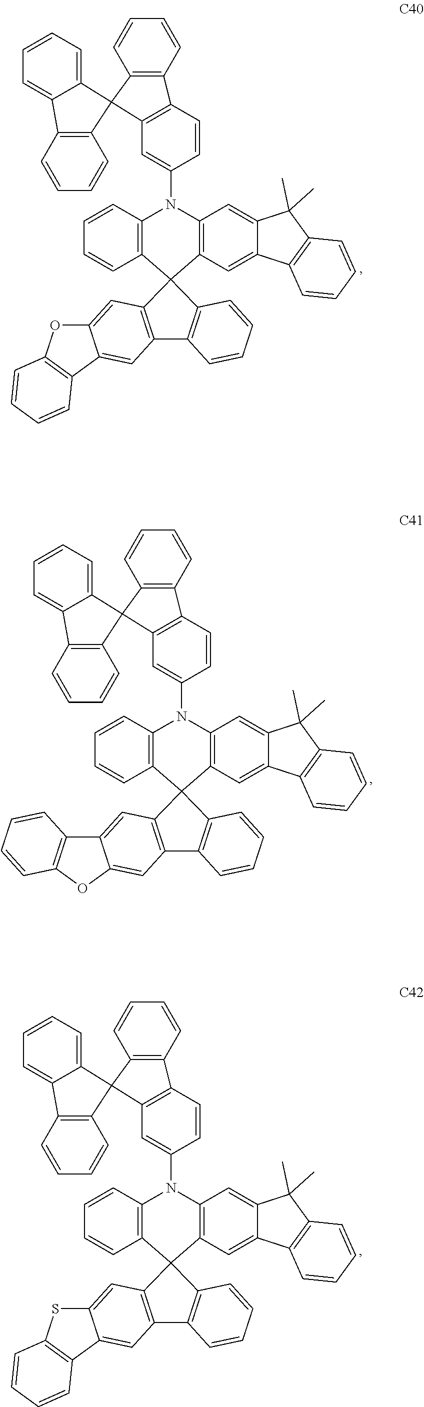 [SMILES: CC1(C)c2ccccc2-c2cc3c(cc21)N(c1ccc2c(c1)C1(c4ccccc4-c4ccccc41)c1ccccc1-2)c1ccccc1C31c2ccccc2-c2cc3c(cc21)oc1ccccc13 ; CC1(C)c2ccccc2-c2cc3c(cc21)N(c1ccc2c(c1)C1(c4ccccc4-c4ccccc41)c1ccccc1-2)c1ccccc1C31c2ccccc2-c2cc3oc4ccccc4c3cc21 ; CC1(C)c2ccccc2-c2cc3c(cc21)N(c1ccc2c(c1)C1(c4ccccc4-c4ccccc41)c1ccccc1-2)c1ccccc1C31c2ccccc2-c2cc3c(cc21)sc1ccccc13] 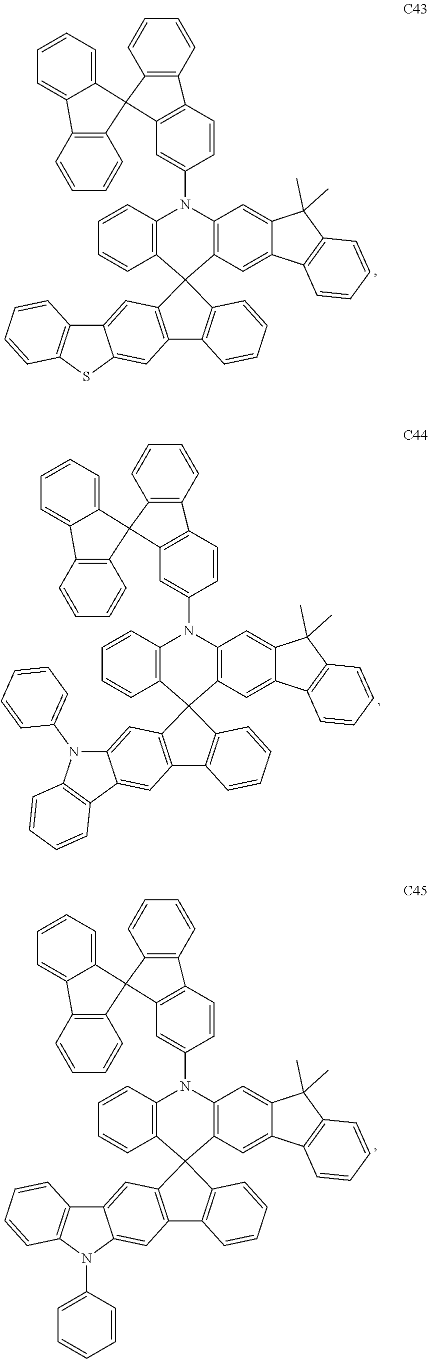 [SMILES: CC1(C)c2ccccc2-c2cc3c(cc21)N(c1ccc2c(c1)C1(c4ccccc4-c4ccccc41)c1ccccc1-2)c1ccccc1C31c2ccccc2-c2cc3sc4ccccc4c3cc21 ; CC1(C)c2ccccc2-c2cc3c(cc21)N(c1ccc2c(c1)C1(c4ccccc4-c4ccccc41)c1ccccc1-2)c1ccccc1C31c2ccccc2-c2cc3c4ccccc4n(-c4ccccc4)c3cc21 ; CC1(C)c2ccccc2-c2cc3c(cc21)N(c1ccc2c(c1)C1(c4ccccc4-c4ccccc41)c1ccccc1-2)c1ccccc1C31c2ccccc2-c2cc3c(cc21)c1ccccc1n3-c1ccccc1] 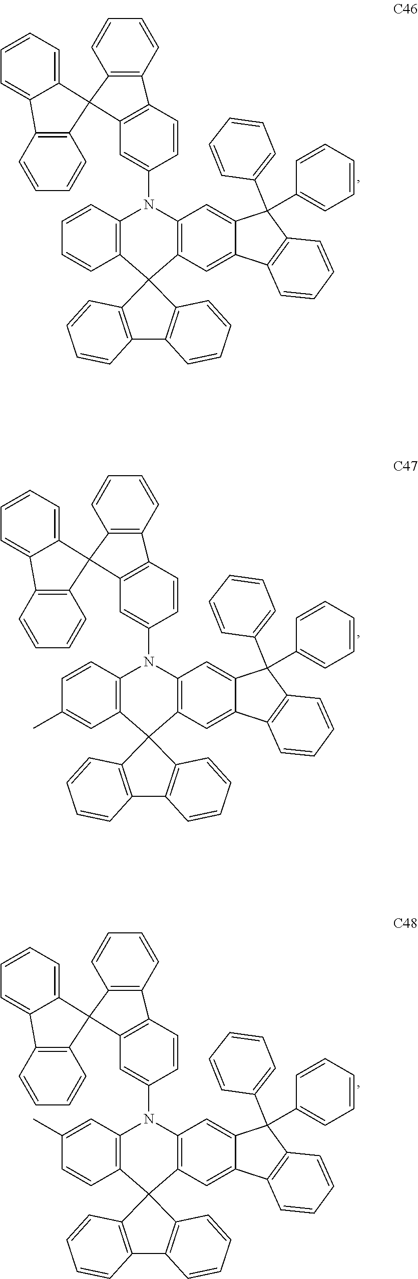 [SMILES: c1ccc(C2(c3ccccc3)c3ccccc3-c3cc4c(cc32)N(c2ccc3c(c2)C2(c5ccccc5-c5ccccc52)c2ccccc2-3)c2ccccc2C42c3ccccc3-c3ccccc32)cc1 ; Cc1ccc2c(c1)C1(c3ccccc3-c3ccccc31)c1cc3c(cc1N2c1ccc2c(c1)C1(c4ccccc4-c4ccccc41)c1ccccc1-2)C(c1ccccc1)(c1ccccc1)c1ccccc1-3 ; Cc1ccc2c(c1)N(c1ccc3c(c1)C1(c4ccccc4-c4ccccc41)c1ccccc1-3)c1cc3c(cc1C21c2ccccc2-c2ccccc21)-c1ccccc1C3(c1ccccc1)c1ccccc1] 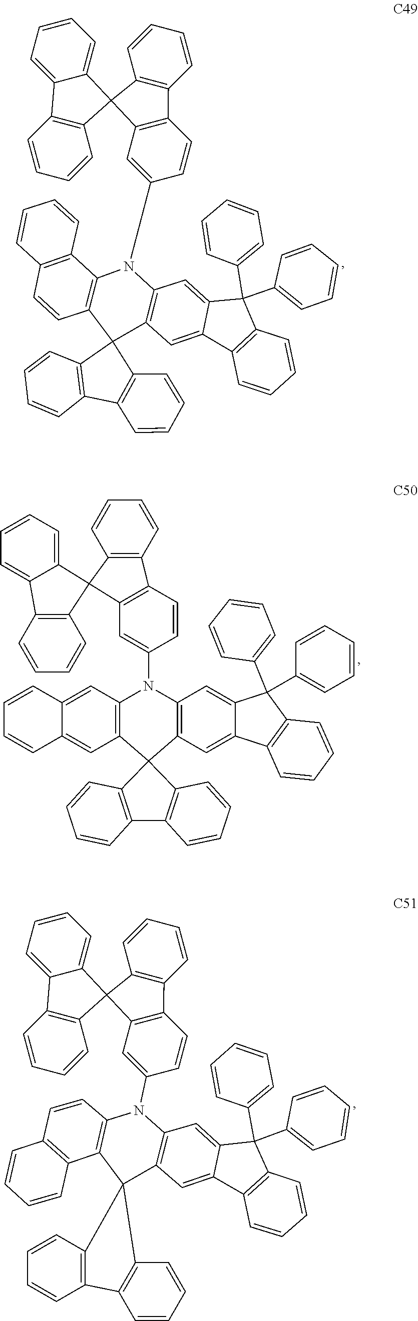 [SMILES: c1ccc(C2(c3ccccc3)c3ccccc3-c3cc4c(cc32)N(c2ccc3c(c2)C2(c5ccccc5-c5ccccc52)c2ccccc2-3)c2c(ccc3ccccc23)C42c3ccccc3-c3ccccc32)cc1 ; c1ccc(C2(c3ccccc3)c3ccccc3-c3cc4c(cc32)N(c2ccc3c(c2)C2(c5ccccc5-c5ccccc52)c2ccccc2-3)c2cc3ccccc3cc2C42c3ccccc3-c3ccccc32)cc1 ; c1ccc(C2(c3ccccc3)c3ccccc3-c3cc4c(cc32)N(c2ccc3c(c2)C2(c5ccccc5-c5ccccc52)c2ccccc2-3)c2ccc3ccccc3c2C42c3ccccc3-c3ccccc32)cc1] 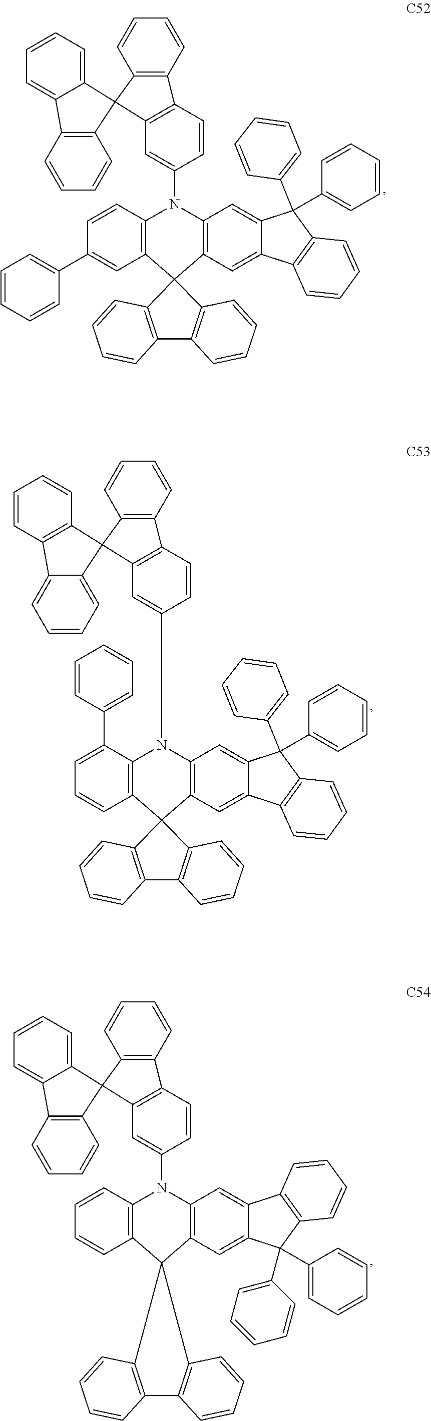 [SMILES: c1ccc(-c2ccc3c(c2)C2(c4ccccc4-c4ccccc42)c2cc4c(cc2N3c2ccc3c(c2)C2(c5ccccc5-c5ccccc52)c2ccccc2-3)C(c2ccccc2)(c2ccccc2)c2ccccc2-4)cc1 ; c1ccc(-c2cccc3c2N(c2ccc4c(c2)C2(c5ccccc5-c5ccccc52)c2ccccc2-4)c2cc4c(cc2C32c3ccccc3-c3ccccc32)-c2ccccc2C4(c2ccccc2)c2ccccc2)cc1 ; c1ccc(C2(c3ccccc3)c3ccccc3-c3cc4c(cc32)C2(c3ccccc3-c3ccccc32)c2ccccc2N4c2ccc3c(c2)C2(c4ccccc4-c4ccccc42)c2ccccc2-3)cc1] 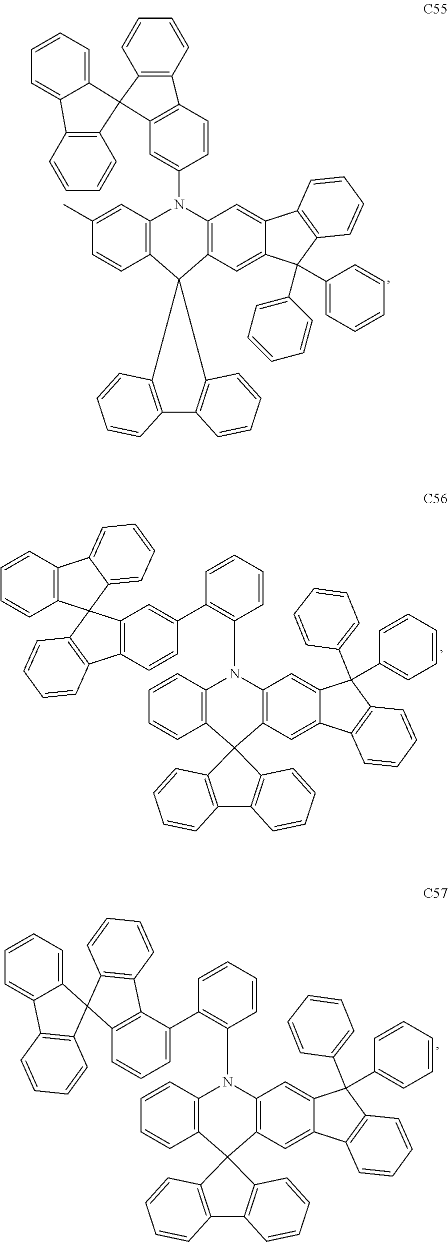 [SMILES: Cc1ccc2c(c1)N(c1ccc3c(c1)C1(c4ccccc4-c4ccccc41)c1ccccc1-3)c1cc3c(cc1C21c2ccccc2-c2ccccc21)C(c1ccccc1)(c1ccccc1)c1ccccc1-3 ; c1ccc(C2(c3ccccc3)c3ccccc3-c3cc4c(cc32)N(c2ccccc2-c2ccc3c(c2)C2(c5ccccc5-c5ccccc52)c2ccccc2-3)c2ccccc2C42c3ccccc3-c3ccccc32)cc1 ; c1ccc(C2(c3ccccc3)c3ccccc3-c3cc4c(cc32)N(c2ccccc2-c2cccc3c2-c2ccccc2C32c3ccccc3-c3ccccc32)c2ccccc2C42c3ccccc3-c3ccccc32)cc1] 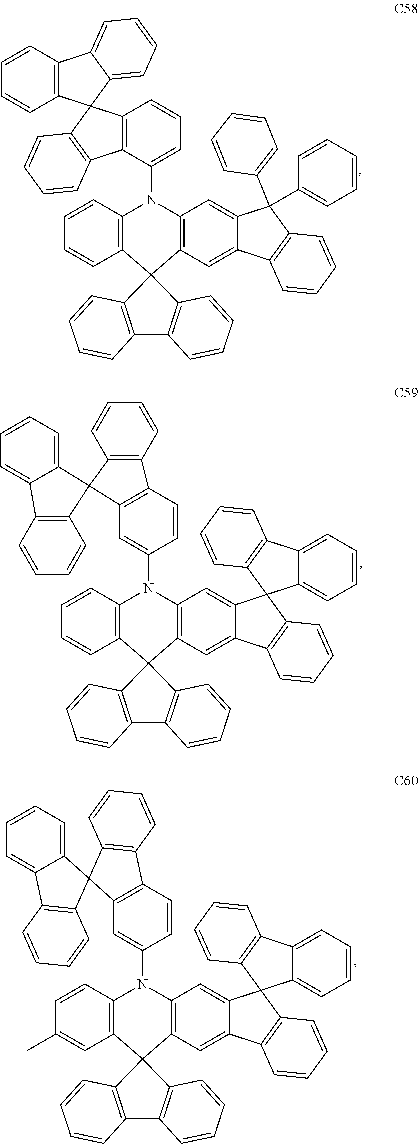 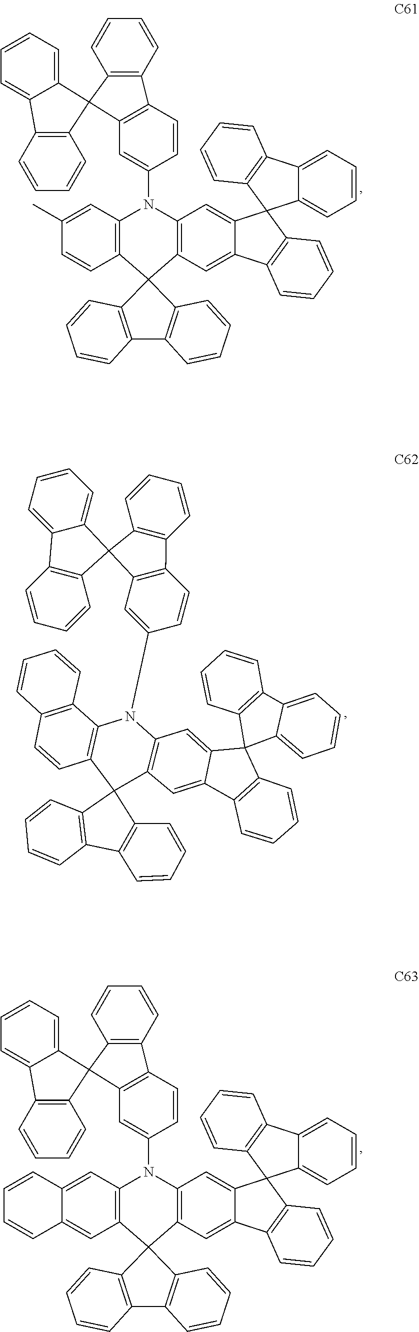 [SMILES: Cc1ccc2c(c1)N(c1ccc3c(c1)C1(c4ccccc4-c4ccccc41)c1ccccc1-3)c1cc3c(cc1C21c2ccccc2-c2ccccc21)-c1ccccc1C31c2ccccc2-c2ccccc21 ; c1ccc2c(c1)-c1ccccc1C21c2ccccc2-c2ccc(N3c4cc5c(cc4C4(c6ccccc6-c6ccccc64)c4ccc6ccccc6c43)-c3ccccc3C53c4ccccc4-c4ccccc43)cc21 ; c1ccc2c(c1)-c1ccccc1C21c2ccccc2-c2ccc(N3c4cc5c(cc4C4(c6ccccc6-c6ccccc64)c4cc6ccccc6cc43)-c3ccccc3C53c4ccccc4-c4ccccc43)cc21] 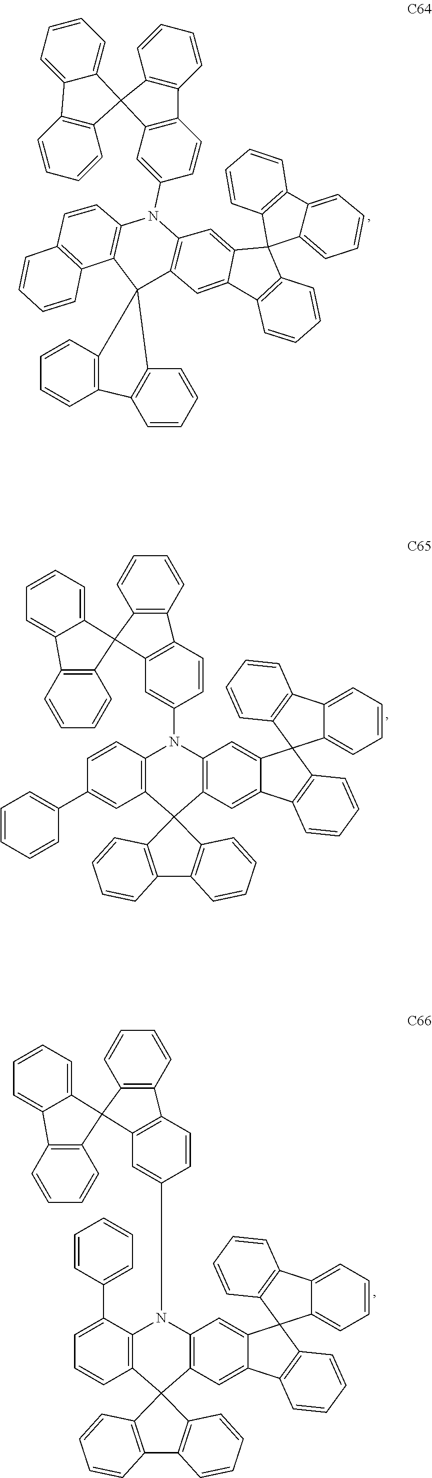 [SMILES: c1ccc2c(c1)-c1ccccc1C21c2ccccc2-c2ccc(N3c4cc5c(cc4C4(c6ccccc6-c6ccccc64)c4c3ccc3ccccc43)-c3ccccc3C53c4ccccc4-c4ccccc43)cc21 ; c1ccc(-c2ccc3c(c2)C2(c4ccccc4-c4ccccc42)c2cc4c(cc2N3c2ccc3c(c2)C2(c5ccccc5-c5ccccc52)c2ccccc2-3)C2(c3ccccc3-c3ccccc32)c2ccccc2-4)cc1 ; c1ccc(-c2cccc3c2N(c2ccc4c(c2)C2(c5ccccc5-c5ccccc52)c2ccccc2-4)c2cc4c(cc2C32c3ccccc3-c3ccccc32)-c2ccccc2C42c3ccccc3-c3ccccc32)cc1] 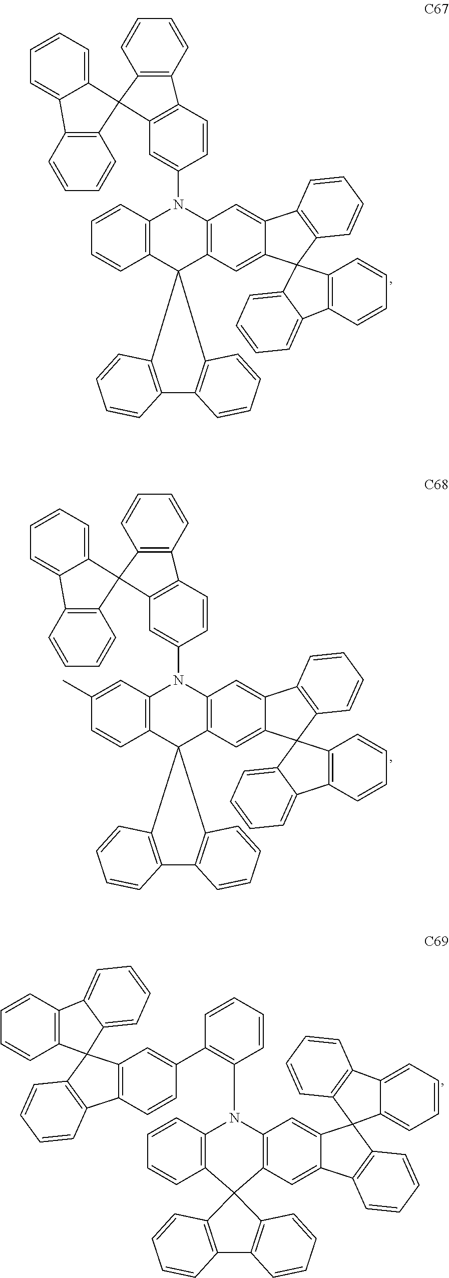 [SMILES: c1ccc2c(c1)-c1ccccc1C21c2ccccc2-c2ccc(N3c4ccccc4C4(c5ccccc5-c5ccccc54)c4cc5c(cc43)-c3ccccc3C53c4ccccc4-c4ccccc43)cc21 ; Cc1ccc2c(c1)N(c1ccc3c(c1)C1(c4ccccc4-c4ccccc41)c1ccccc1-3)c1cc3c(cc1C21c2ccccc2-c2ccccc21)C1(c2ccccc2-c2ccccc21)c1ccccc1-3 ; c1ccc(N2c3ccccc3C3(c4ccccc4-c4ccccc43)c3cc4c(cc32)C2(c3ccccc3-c3ccccc32)c2ccccc2-4)c(-c2ccc3c(c2)C2(c4ccccc4-c4ccccc42)c2ccccc2-3)c1] 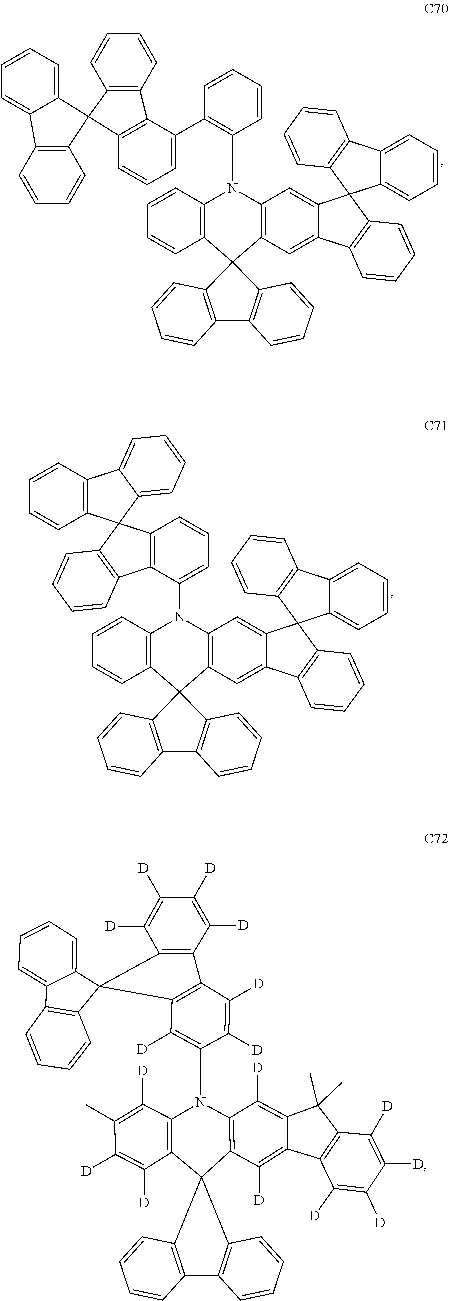 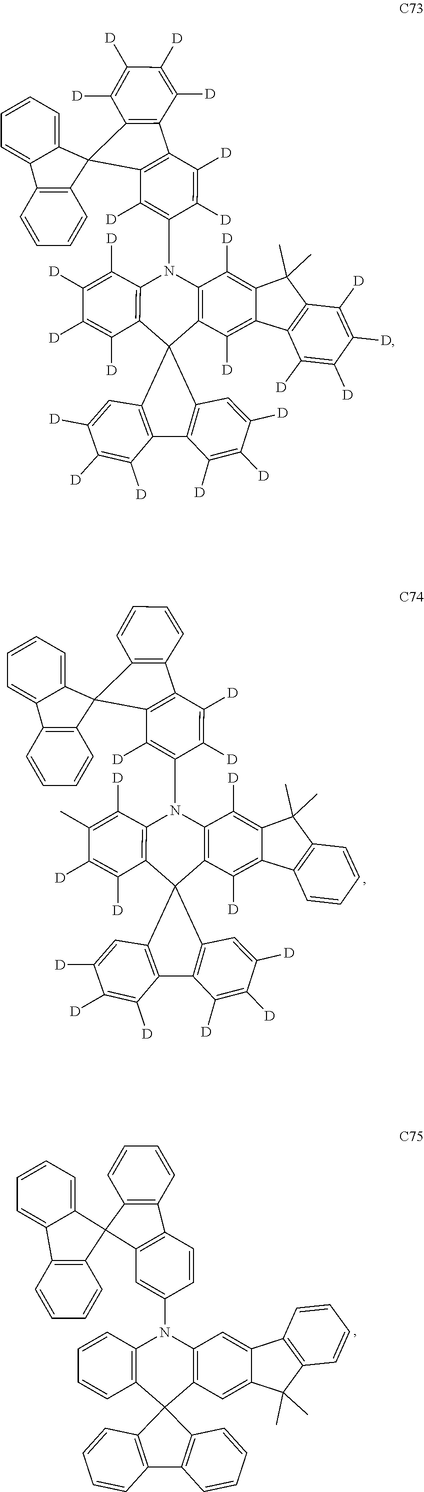 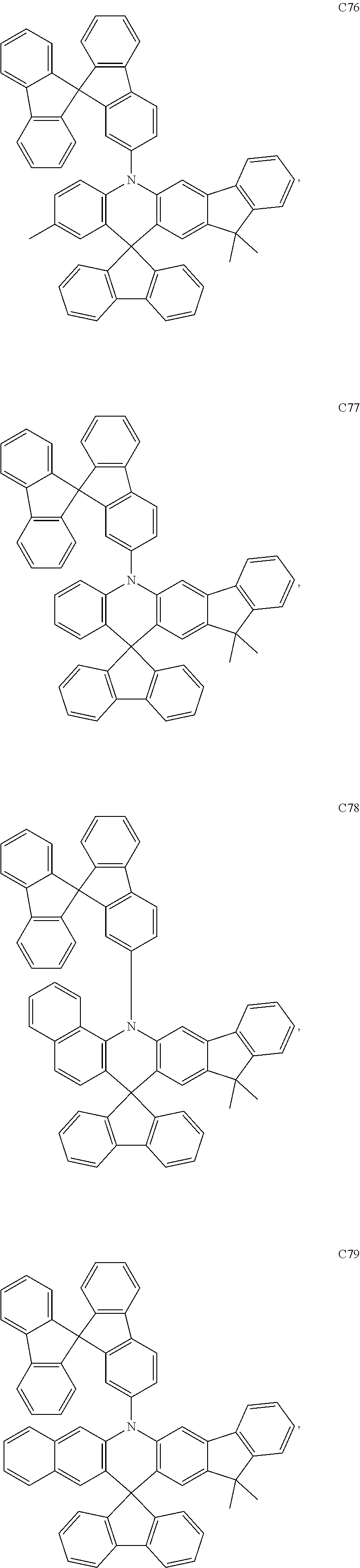 [SMILES: Cc1ccc2c(c1)C1(c3ccccc3-c3ccccc31)c1cc3c(cc1N2c1ccc2c(c1)C1(c4ccccc4-c4ccccc41)c1ccccc1-2)-c1ccccc1C3(C)C ; CC1(C)c2ccccc2-c2cc3c(cc21)C1(c2ccccc2-c2ccccc21)c1ccccc1N3c1ccc2c(c1)C1(c3ccccc3-c3ccccc31)c1ccccc1-2 ; CC1(C)c2ccccc2-c2cc3c(cc21)C1(c2ccccc2-c2ccccc21)c1ccc2ccccc2c1N3c1ccc2c(c1)C1(c3ccccc3-c3ccccc31)c1ccccc1-2 ; CC1(C)c2ccccc2-c2cc3c(cc21)C1(c2ccccc2-c2ccccc21)c1cc2ccccc2cc1N3c1ccc2c(c1)C1(c3ccccc3-c3ccccc31)c1ccccc1-2] 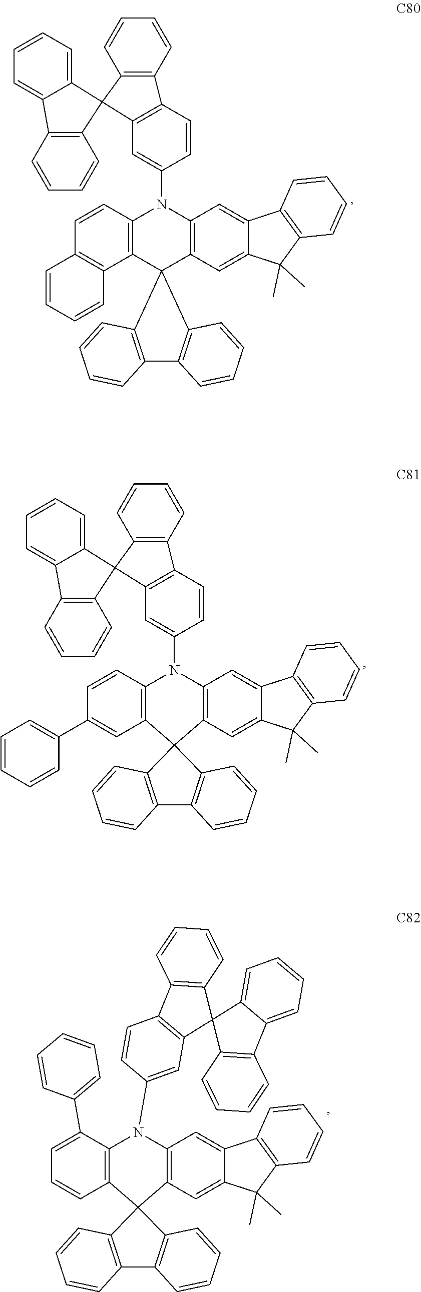 [SMILES: CC1(C)c2ccccc2-c2cc3c(cc21)C1(c2ccccc2-c2ccccc21)c1c(ccc2ccccc12)N3c1ccc2c(c1)C1(c3ccccc3-c3ccccc31)c1ccccc1-2 ; CC1(C)c2ccccc2-c2cc3c(cc21)C1(c2ccccc2-c2ccccc21)c1cc(-c2ccccc2)ccc1N3c1ccc2c(c1)C1(c3ccccc3-c3ccccc31)c1ccccc1-2 ; CC1(C)c2ccccc2-c2cc3c(cc21)C1(c2ccccc2-c2ccccc21)c1cccc(-c2ccccc2)c1N3c1ccc2c(c1)C1(c3ccccc3-c3ccccc31)c1ccccc1-2] 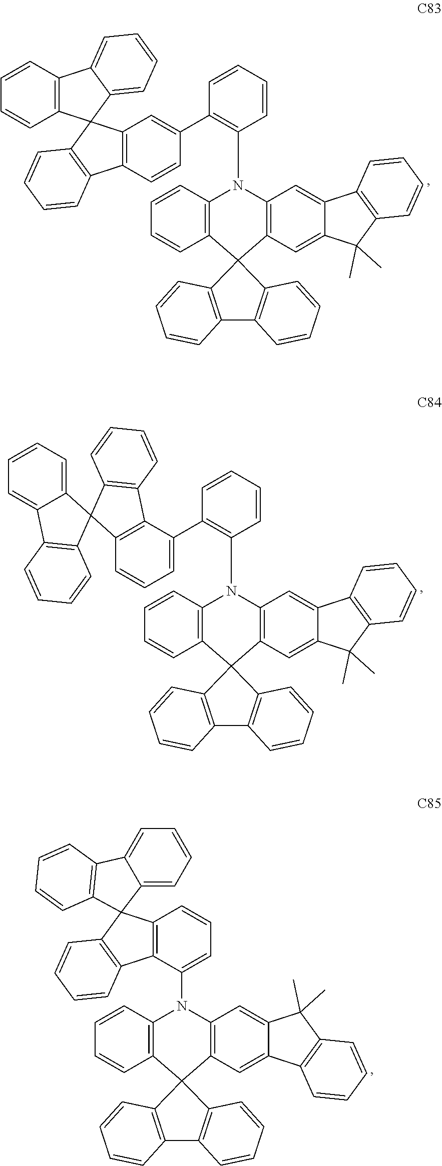 [SMILES: CC1(C)c2ccccc2-c2cc3c(cc21)C1(c2ccccc2-c2ccccc21)c1ccccc1N3c1ccccc1-c1ccc2c(c1)C1(c3ccccc3-c3ccccc31)c1ccccc1-2 ; CC1(C)c2ccccc2-c2cc3c(cc21)C1(c2ccccc2-c2ccccc21)c1ccccc1N3c1ccccc1-c1cccc2c1-c1ccccc1C21c2ccccc2-c2ccccc21 ; CC1(C)c2ccccc2-c2cc3c(cc21)N(c1cccc2c1-c1ccccc1C21c2ccccc2-c2ccccc21)c1ccccc1C31c2ccccc2-c2ccccc21] 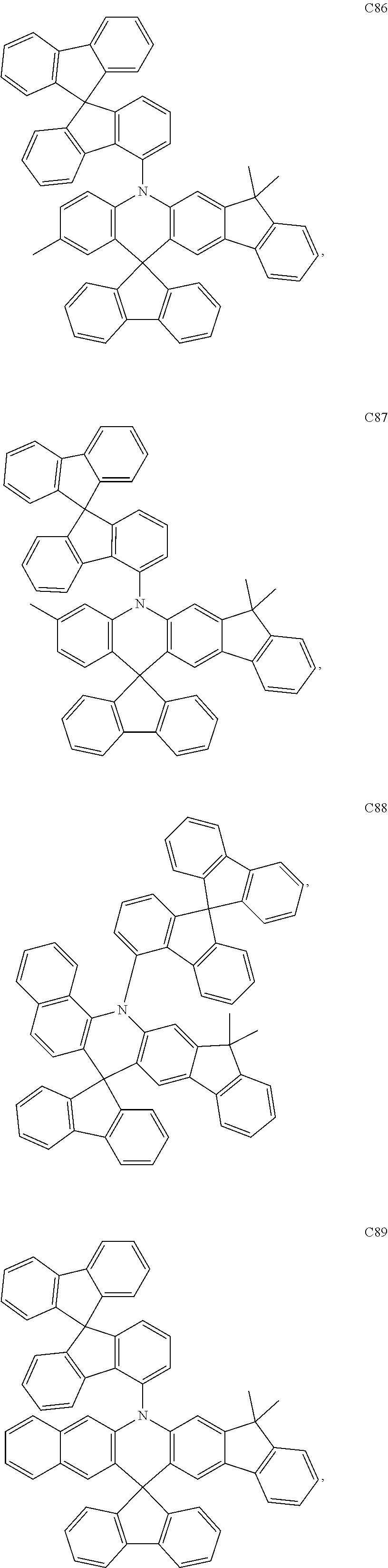 [SMILES: Cc1ccc2c(c1)C1(c3ccccc3-c3ccccc31)c1cc3c(cc1N2c1cccc2c1-c1ccccc1C21c2ccccc2-c2ccccc21)C(C)(C)c1ccccc1-3 ; Cc1ccc2c(c1)N(c1cccc3c1-c1ccccc1C31c3ccccc3-c3ccccc31)c1cc3c(cc1C21c2ccccc2-c2ccccc21)-c1ccccc1C3(C)C ; CC1(C)c2ccccc2-c2cc3c(cc21)N(c1cccc2c1-c1ccccc1C21c2ccccc2-c2ccccc21)c1c(ccc2ccccc12)C31c2ccccc2-c2ccccc21 ; CC1(C)c2ccccc2-c2cc3c(cc21)N(c1cccc2c1-c1ccccc1C21c2ccccc2-c2ccccc21)c1cc2ccccc2cc1C31c2ccccc2-c2ccccc21] 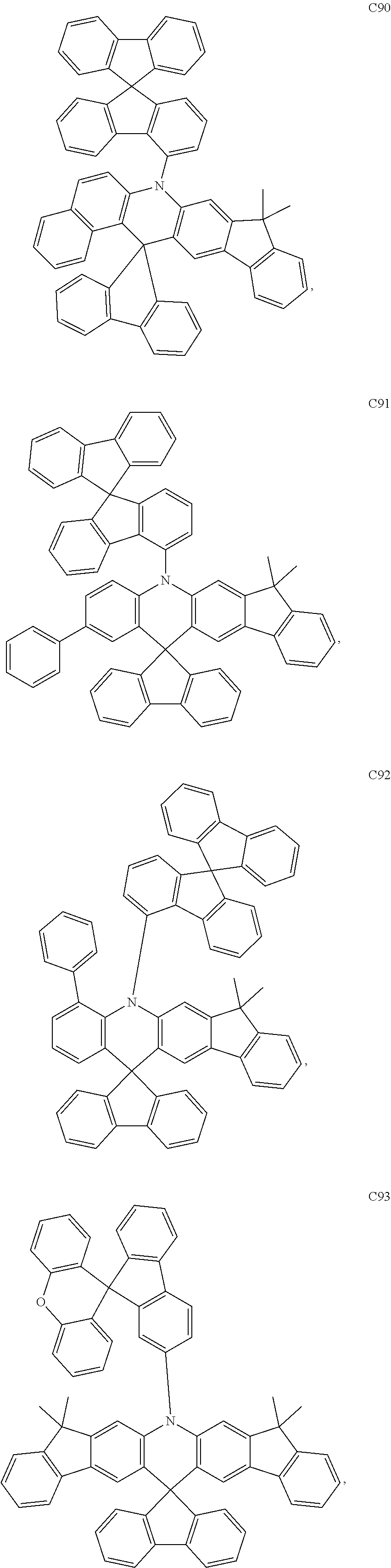 [SMILES: CC1(C)c2ccccc2-c2cc3c(cc21)N(c1cccc2c1-c1ccccc1C21c2ccccc2-c2ccccc21)c1ccc2ccccc2c1C31c2ccccc2-c2ccccc21 ; CC1(C)c2ccccc2-c2cc3c(cc21)N(c1cccc2c1-c1ccccc1C21c2ccccc2-c2ccccc21)c1ccc(-c2ccccc2)cc1C31c2ccccc2-c2ccccc21 ; CC1(C)c2ccccc2-c2cc3c(cc21)N(c1cccc2c1-c1ccccc1C21c2ccccc2-c2ccccc21)c1c(-c2ccccc2)cccc1C31c2ccccc2-c2ccccc21 ; CC1(C)c2ccccc2-c2cc3c(cc21)N(c1ccc2c(c1)C1(c4ccccc4Oc4ccccc41)c1ccccc1-2)c1cc2c(cc1C31c3ccccc3-c3ccccc31)-c1ccccc1C2(C)C] 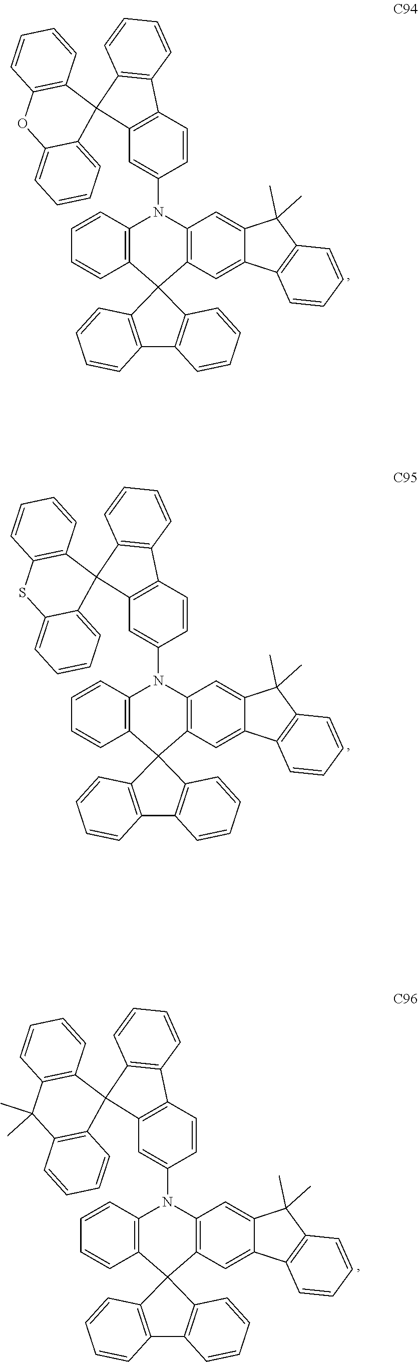 [SMILES: CC1(C)c2ccccc2-c2cc3c(cc21)N(c1ccc2c(c1)C1(c4ccccc4Oc4ccccc41)c1ccccc1-2)c1ccccc1C31c2ccccc2-c2ccccc21 ; CC1(C)c2ccccc2-c2cc3c(cc21)N(c1ccc2c(c1)C1(c4ccccc4Sc4ccccc41)c1ccccc1-2)c1ccccc1C31c2ccccc2-c2ccccc21 ; CC1(C)c2ccccc2-c2cc3c(cc21)N(c1ccc2c(c1)C1(c4ccccc4-2)c2ccccc2C(C)(C)c2ccccc21)c1ccccc1C31c2ccccc2-c2ccccc21] 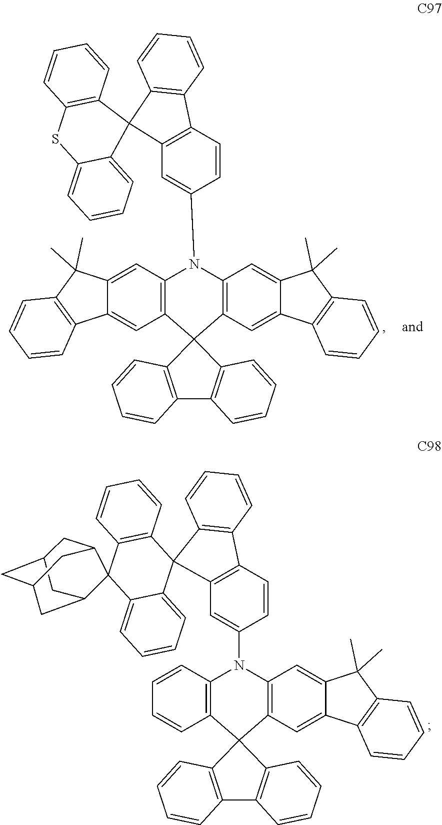 [SMILES: CC1(C)c2ccccc2-c2cc3c(cc21)N(c1ccc2c(c1)C1(c4ccccc4Sc4ccccc41)c1ccccc1-2)c1cc2c(cc1C31c3ccccc3-c3ccccc31)-c1ccccc1C2(C)C ; CC1(C)c2ccccc2-c2cc3c(cc21)N(c1ccc2c(c1)C1(c4ccccc4-2)c2ccccc2C2(c4ccccc41)C1CC4CC(C1)CC2C4)c1ccccc1C31c2ccccc2-c2ccccc21]
wherein optionally, hydrogens in structures of Compound A1 to Compound A124, Compound B1 to Compound B92, and Compound C1 to Compound C98 can be partially or fully substituted with deuterium.

11. An organic electroluminescent device, comprising:
an anode,
a cathode, and
an organic layer disposed between the anode and the cathode, wherein the organic layer comprises the compound of claim 1.

12. The organic electroluminescent device of claim 11, wherein the organic layer is an electron blocking layer, a hole injection layer, a hole transport layer or a light-emitting layer; and
preferably, the organic layer is an electron blocking layer, and the compound is an electron blocking material.

13. A compound composition, comprising the compound of claim 1.
